SEQUENCE LISTING (3 sequences)

SEQ ID NO: 1 - protein, 12 aa, Homo sapiens
RPRLSHKGPMPF

SEQ ID NO: 2 - protein, 13 aa, Homo sapiens
QRPRLSHKGPMPF

SEQ ID NO: 3 - protein, 36 aa, Homo sapiens
LVQPRGSRNGPGPWQGGRRKFRRQRPRLSHKGPMPF

(19) United States
(12) Patent Application Publication
(10) Pub. No.: US 2005/0152836 A1
(43) Pub. Date: Jul. 14, 2005

(54) Diagnostic markers and pharmacological targets in heart failure and related reagents and methods of use thereof

(76) Inventors: Euan Ashley, Palo Alto, CA (US); Mary M. Chen, Fremont, CA (US); Thomas Quertermous, Stanford, CA (US); David Xing-Fei Deng, Mountain View, CA (US); Anya Tsalenko, Chicago, IL (US); Amir Ben-dor, Bellevue, WA (US); Laurakay Bruhn, Mountain View, CA (US); Zohar Yakhini, Ramat HaSharon (IL)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/850,941

(22) Filed: May 21, 2004

Related U.S. Application Data
(60) Provisional application No. 60/472,619, filed on May 22, 2003.

(51) Int. Cl. A61K 51/00; C12Q 1/68; G01N 33/53; G01N 33/567; C07K 16/46

(52) U.S. Cl. 424/1.49; 435/6; 435/7.2; 530/391.1

(57) ABSTRACT

The invention identifies genes whose expression is upregulated or downregulated following mechanical offloading in subjects with heart failure. The invention provides compositions comprising a targeting agent conjugated to a functional moiety, wherein the targeting agent selectively binds to a polypeptide encoded by one of these genes. The functional moiety can be an imaging agent, therapeutic agent, etc. The invention further provides methods for providing diagnostic or prognostic information related to heart failure involving detecting expression or activity of an expression product of one or more of the identified genes. The invention further provides diagnostic and therapeutic methods comprising detecting or administering an apelin peptide to a subject.

### CROSS-REFERENCE TO RELATED APPLICATION

This application claims priority to U.S. Provisional Patent Application 60/472,619, filed May 22, 2003, which is incorporated herein by reference.

### BACKGROUND OF THE INVENTION

Heart failure is a pathophysiological state in which the heart is unable to pump enough blood to meet the nutrition and oxygen requirement of metabolizing tissues or cells. It is a major complication in many heart diseases. Adults over the age of 40 have an estimated 21% lifetime risk of developing heart failure (Lloyd-Jones, D. M. et al. Lifetime risk for developing congestive heart failure: the Framingham Heart Study. Circulation 106, 3068-72 (2002), a condition responsible for more hospitalizations than all forms of cancer combined (American Heart Association. Heart Disease and Stroke Statistics—2003 Update, (American Heart Association, Dallas, Tex., 2003)).

Heart failure is a general term that describes the final common pathway of many disease processes. The most common cause is coronary artery disease, which can lead to a myocardial infarction (heart attack), often resulting in death of cardiac cells. The heart must then perform the same work with fewer cells. Chronic obstructive coronary artery disease can also cause heart failure in the absence of myocardial infarction. Valve disease or high blood pressure can lead to heart failure by increasing the workload of the heart. Rarer causes of heart failure, which primarily involve cardiac muscle, are classed as cardiomyopathy (although this term is sometimes used more generally to cover any cause of heart failure). The best characterized are a group of single gene disorders of the sarcomere which cause “hypertrophic cardiomyopathy” (in fact, a misnomer as many patients have no hypertrophy). In contrast, all patients with “dilated cardiomyopathy” have dilated thin walled ventricles. The genetics of this condition have yet to be characterized, but in many cases non-genetic causes are responsible (e.g. infections, alcohol, chemotherapeutic agents). Where no readily identifiable cause is found, the diagnosis used is ‘idiopathic’ dilated cardiomyopathy (generally a diagnosis of exclusion).

A variety of pathophysiological changes occur in the heart as heart failure develops. In response to increased work load in vivo, the heart frequently increases in size (cardiac hypertrophy) as cardiac muscle cells develop hypertrophy (i.e., an increase in cell size in the absence of cell division). At the cellular and molecular levels, cardiac hypertrophy is characterized by increased expression of contractile proteins and activation of various signaling pathways whose role in the pathophysiology of heart failure remains incompletely understood.

Current treatments for heart failure include pharmacological methods, devices such as the ventricular assist device (VAD), and heart and heart-lung transplantation. Pharmacological approaches include the use of inotropic agents (i.e., compounds that increase cardiac contractility), neurohumoral blockers (e.g., β-blockers, angiotensin converting enzyme inhibitors), aldosterone antagonists, diuretics, and vasodilators. However, none of these agents is fully effective either alone or in combination. Availability of transplants is limited, and since many individuals suffering from heart failure are in poor health, they are frequently not good surgical candidates. For these reasons heart failure remains a major cause of morbidity and mortality, particularly in the developed world. In addition, as indicated above it can be difficult to determine the etiology of heart failure, thus impeding the development of more specific therapies. In addition, there is a lack of diagnostic techniques at the molecular level. Thus there is a need in the art for the discovery of additional diagnostic markers and pharmacological targets for the development of new therapeutic approaches. In addition, there is a need in the art for improved techniques for evaluating the severity of heart failure and its response to treatment. The present invention addresses the foregoing needs, among others.

### SUMMARY OF THE INVENTION

The present invention employs microarray analysis to identify genes whose expression is either upregulated (i.e., increases) or downregulated (i.e., decreases) after mechanical offloading of the heart in patients with heart failure. Without wishing to be bound by any theory, in accordance with the invention mechanical offloading represents a state of recovery from heart failure. Genes whose expresson is upregulated following mechanical offloading are therefore referred to herein as “upregulated in recovery” (UIR) genes, and genes whose expression is downregulated following mechanical offloading are referred to herein as “down-regulated in recovery” (DIR) genes. Polypeptides encoded by these genes are referred to as UIR and DIR polypeptides, respectively. The invention provides methods for identification of genes that are diagnostic and/or therapeutic targets in heart failure.

Related to the identification of these genes, the invention provides a composition comprising a targeting agent conjugated to a functional moiety, wherein the targeting agent selectively binds to a polypeptide encoded by a UIR or DIR gene. In certain embodiments of the invention the UIR or DIR gene encodes a polypeptide selected from the group consisting of: APJ, mitogen-activated protein kinase 4, the TEC protein tyrosine kinase, the P311 protein, guanylate binding protein 1, tumor necrosis factor (ligand) superfamily member 10, a splice variant of the regulatory domain (α subunit) of the L-type calcium channel, myosin light chain kinase pseudogene expression product, and myosin light chain 2a. The targeting agent may comprise an antibody, an antigen-binding antibody fragment, or a ligand that specifically binds to the polypeptide. Preferred functional moieties include imaging agents and therapeutic agents.

The invention further provides a method of imaging cardiac tissue comprising steps of: (i) administering to a subject an effective amount of a targeting agent that specifically binds to a UIR or DIR polypeptide, wherein the targeting agent is linked to a functional moiety that enhances detectability of cardiac cells by an imaging procedure; and (ii) subjecting the subject to the imaging procedure.

The invention additionally provides a method of targeting a molecule selectively to a cardiac cell in culture or in a subject comprising steps of: (i) conjugating the molecule to an antibody or ligand that specifically binds to a DIR polypeptide to form a conjugate; and (ii) administering the conjugate to the cell or to the subject. The invention also provides a second method of targeting a molecule selectively to a cardiac cell in culture or in a subject comprising steps of: (i) associating the molecule with a delivery vehicle, wherein the delivery vehicle comprises a targeting agent that specifically binds to a UIR or DIR polypeptide; and (ii) administering the delivery vehicle to the cell or subject.

In another aspect, the invention provides a method for identifying an agent that modulates expression or activity of a UIR or DIR polynucleotide or polypeptide comprising steps of: (i) providing a sample comprising a UIR or DIR polynucleotide or polypeptide; (ii) contacting the sample with a candidate compound; (iii) determining whether the level of expression or activity of the polynucleotide or polypeptide in the presence of the compound is increased or decreased relative to the level of expression or activity of the polynucleotide or polypeptide in the absence of the compound; and (iv) identifying the compound as a modulator of the expression or activity of the UIR or DIR polynucleotide or polypeptide if the level of expression or activity of the UIR or DIR polynucleotide or polypeptide is higher or lower in the presence of the compound relative to its level of expression or activity in the absence of the compound.

The invention further provides a method of providing diagnostic or prognostic information related to heart failure comprising steps of: (i) providing a subject in need of diagnostic or prognostic information related to heart failure; (ii) determining the level of expression or activity of a UIR or DIR polynucleotide or polypeptide, or the level of a ligand for a UIR or DIR polypeptide, in the subject or in a biological sample obtained from the subject; and (iii) utilizing the information to provide diagnostic or prognostic information. In a preferred embodiment of this method the polypeptide is an apelin peptide.

In another aspect, the invention provides a method of treating or preventing heart failure or a disease or condition associated with heart failure comprising steps of: (i) providing a subject at risk of or suffering from heart failure or a disease or condition associated with heart failure; and (ii) administering a composition that modulates a UIR or DIR gene. In a preferred embodiment of the invention the composition increases the functional activity of the polypeptide known as APJ.

The invention also provides a method of treating or preventing heart failure or a disease or condition associated with heart failure comprising steps of: (i) providing a subject at risk of or suffering from heart failure or a disease or condition associated with heart failure; and (ii) administering a composition comprising an apelin peptide to the subject. According to certain embodiments of the invention the apelin peptide is administered chronically. In certain embodiments of the invention the apelin peptide is administered in an amount effective to improve at least one hemodynamic parameter or prognostic variable for heart failure.

This application refers to various patents, patent applications, journal articles, and other publications, all of which are incorporated herein by reference. In addition, the following standard reference works are incorporated herein by reference: Current Protocols in Molecular Biology, Current Protocols in Immunology, Current Protocols in Protein Science, and Current Protocols in Cell Biology, John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001; Rodd 1989 “Chemistry of Carbon Compounds”, vols. 1-5 and supps, Elsevier Science Publishers, 1989; “Organic Reactions”, vols 1-40, John Wiley and Sons, New York, N.Y., 1991; March 2001, “Advanced Organic Chemistry”, 5th ed. John Wiley and Sons, New York, N.Y., Braunwald, E., Zipes, D. P., and Libby, P. (eds.) Heart Disease: A Textbook of Cardiovascular Medicine. W B Saunders; 6th edition (Feb. 15, 2001); Chien, K. R., Molecular Basis of Cardiovascular Disease: A Companion to Braunwald's Heart Disease, W B Saunders; Revised edition (2003); and Goodman and Gilman's The Pharmacological Basis of Therapeutics, 10^{th} Ed. McGraw Hill, 2001 (referred to herein as Goodman and Gilman). In case of conflict between the instant specification and any document incorporated by reference, the specification shall control.

### BRIEF DESCRIPTION OF THE DRAWING

FIGS. 1A-1D show a microarray analysis of cardiac gene expression before and after insertion of a left ventricular assist device (LVAD). FIG. 1A is a heatmap representation of genes significantly differentially regulated following LVAD. Rows represent individual genes, columns represent patients. Color intensity values are row-normalized and rows are ordered according to the di statistic of the Significance Analysis of Microarrays (SAM). FIG. 1B shows a dendrogram output of average-linkage hierarchical clustering analysis generated using Cluster and Treeview software (Eisen, M. B., Spellman, P. T., Brown, P. O. & Botstein, D. Cluster analysis and display of genome-wide expression patterns. Proc Natl Acad Sci U S A 95, 14863-8, 1998). Color saturation values represent absolute gene expression across all genes. FIG. 1C shows a dendrogram output of average-linkage hierarchical clustering for all patients pre and post LVAD. FIG. 1D is a plot showing validation of microarray findings using quantitative real time reverse transcription polymerase chain reaction (qRt-PCR). Fold changes for five genes across seven patients as determined by hybridization are plotted against those determined by qRT-PCR. Linear regression demonstrates a close relationship between the two variables (R²=0.86).

FIGS. 2A and 2B show apelin level and distribution in human left ventricle. FIG. 2A is a bar graph showing left ventricular tissue apelin level as determined by enzyme immunoassay. The level rose significantly (P<0.001) following offloading by implantation of a left ventricular assist device. Units are ng/ml. FIG. 2B is a tissue section showing immunohistochemical distribution of apelin. Apelin, labeled reddish-browm, is highly localized to endothelial and smooth muscle cells in diseased (right panel) and normal heart (data not shown). Staining of consecutive sections with PECAM (CD31, middle panels) confirms the specificity of this localization. Control panels (left) represent sections where the incubation in primary antibody step was omitted.

FIGS. 3A and 3B show plasma apelin levels in heart failure. FIG. 3A is a bar graph showing that there were significant increases in the plasma level of apelin as determined by enzyme immunoassay in early heart failure through New York Heart Association (NYHA class 2 (P<0.02). In later stage disease, the mean level is lower, although this change is not significant Class 4 patients (n=7) are combined with class 3 (from left to right, n=34, 24, 12, 38). FIG. 3B is a bar graph showing that apelin rises in mild to moderate LV dysfunction but falls in severe disease (P<0.02 for both). Normal is defined as a left ventricular ejection fraction greater than 45%, mild to moderate is 25-45%, and severe is less than 25% (from left to right, n=42,28,40).

FIG. 4 shows immunohistochemistry of apelin and APJ in the developing and adult mouse heart. Specific immunolocalization of both proteins in the developing myocardium revealed very similar patterns of expression as early as embryonic day 13.5 (Panels 4A, 4B: A—atrium, V—ventricle, li—liver). Real time quantitative RT-PCR of isolated heart mRNA suggested that the relative contribution to the total myocardial RNA by these transcripts remains relatively constant through late gestation and adulthood (Panel 4C). In the adult mouse heart, immunolocalization of APJ expression was identified in association with both atrial and ventricular myocardial cells (Panels 4D, 4E). A control slide without the addition of the secondary antibody is shown in Panel 4F.

FIG. 5 shows changes in cardiovascular function following intraperitoneal injection of apelin-12 in C57B16 mice. Mice (n=9) were anesthetized with isoflurance and warmed to 36-37 degrees before magnetic resonance imaging. Electrocardiography revealed a significant increase in HR following apelin injection (Panel 5A). ECG and respiration gated cine magnetic resonance images of the left ventricle taken in short axis reveal a significant reduction in left ventricular end diastolic area (Panel 5C) with an upward trend in ejection fraction (Panel 5B). Panel 5D shows example images of end diastole (left) and end systole (right) from pre (above) and post (below) apelin injection.

FIG. 6 shows pressure-volume hemodynamics in response to acute apelin infusion. Ventilated C57B16 mice underwent placement of a catheter along the long axis of the left ventricle (Panel 6D). Pressure-volume loops including preload reduction facilitated by a 5 second manual occlusion of the inferior vena cava were recorded at baseline (Panel 6A) and following 20 minutes of apelin infusion (Panel 6B). Volume is expressed as relative volume units. After apelin infusion, ventricular elastance was increased (Panel 5C, slope of the end systolic pressure-volume relationship, p=0.0 18) along with preload recruitable stroke work (Panel 6E, p=0.056). Maximum pressure was lower indicating a reduction in afterload (Panel 6F, p=0.02).

FIG. 7 shows the effect of chronic apelin infusion in C57B16 mice. Long axis and short axis views of the left ventricle with Doppler sampling of the outflow tract were used to estimate left ventricular contractility in vivo (Panel 7A). The velocity of circumferential shortening (Panel 7C) and cardiac output (Panel 7D) were significantly increased from baseline following two weeks of PY-apelin-13 infusion. Systolic blood pressure as determined by tail cuff was also lower but this did not reach significance (Panel 7B).

FIG. 8 is a graph showing showing changes in exercise capacity in mice with experimentally induced heart failure from 1-3 weeks during chronic apelin treatment. The graph shows measurements of treadmill time to exhaustion.

### DEFINITIONS

To facilitate understanding of the description of the invention, the following definitions are provided. It is to be understood that, in general, terms not otherwise defined are to be given their meaning or meanings as generally accepted in the art.

Antibody: In general, the term “antibody” refers to an immunoglobulin, which may be natural or wholly or partially synthetically produced in various embodiments of the invention. An antibody may be derived from natural sources (e.g., purified from a rodent, rabbit, chicken (or egg) from an animal that has been immunized with an antigen or a construct that encodes the antigen) partly or wholly synthetically produced. An antibody may be a member of any immunoglobulin class, including any of the human classes: IgG, IgM, IgA, IgD, and IgE. The antibody may be a fragment of an antibody such as an Fab′, F(ab′)₂, scFv (single-chain variable) or other fragment that retains an antigen binding site, or a recombinantly produced scFv fragment, including recombinantly produced fragments. See, e.g., Allen, T., Nature Reviews Cancer, Vol.2, 750-765, 2002, and references therein. Preferred antibodies, antibody fragments, and/or protein domains comprising an antigen binding site may be generated and/or selected in vitro, e.g., using techniques such as phage display (Winter, G. et al.1994. Annu. Rev. Immunol. 12:433-455, 1994), ribosome display (Hanes, J., and Pluckthun, A. Proc. Natl. Acad. Sci. USA. 94:4937-4942, 1997), etc. In various embodiments of the invention the antibody is a “humanized” antibody in which for example, a variable domain of rodent origin is fused to a constant domain of human origin, thus retaining the specificity of the rodent antibody. It is noted that the domain of human origin need not originate directly from a human in the sense that it is first synthesized in a human being. Instead, “human” domains may be generated in rodents whose genome incorporates human immunoglobulin genes. See, e.g., Vaughan, et al., Nature Biotechnology, 16: 535-539, 1998. An antibody may be polyclonal or monoclonal, though for purposes of the present invention monoclonal antibodies are generally preferred.

Cardiac cell: The term “cardiac cell” refers to cardiac myocytes and/or cardiac endothelial cells. According to certain embodiments of the invention the term includes cardiac fibroblasts and/or other cell types present in the heart such as smooth muscle cells (e.g., in the walls of cardiac blood vessels), neurons and glial cells in cardiac nerves, etc.

Diagnostic information: As used herein, “diagnostic information” or information for use in diagnosis is any information that is useful in determining whether a subject has or is susceptible to developing a disease or condition and/or in classifying the disease or condition into a phenotypic category or any category having significance with regards to the prognosis of or likely response to treatment of the disease or condition. The term includes prenatal diagnosis, i.e., diagnosis performed prior to the birth of the subject, including performing genetic testing on germ cells (ova and/or sperm). The term also includes determining the genotype of a subject with respect to a UIR or DIR gene for any purpose.

Diagnostic target: A gene is considered to be a “diagnostic target” if detection and/or measurement of its expression level is useful in providing diagnostic or prognostic information related to a disease or clinical condition, or for monitoring the physiological state of a cell, tissue, or organism (including monitoring the response to therapy or the progression of disease). Expression products of such genes (RNA or polypeptide) may also be referred to as diagnostic targets.

Differential expression: A gene or cDNA clone exhibits “differential expression” at the RNA level if its RNA transcript varies in abundance between different cell types, tissues, samples, etc., at different times, or under different conditions. A gene exhibits differential expression at the protein level if a polypeptide encoded by the gene or cDNA clone varies in abundance between different cell types, tissues, samples, etc., or at different times. In the context of a microarray experiment, differential expression generally refers to differential expression at the RNA level. Differential expression, as used herein, may refer to both quantitative as well as qualitative differences in the temporal and/or tissue expression patterns. In general, differentially expressed genes may be used to identify or detect particular cell types, tissues, physiological states, etc., to distinguish between different cell types, tissues, or physiological states. Differentially expressed genes and their expression products may be diagnostic and/or therapeutic targets or may interact with such targets.

Effective amount: In general, an “effective amount” of an active agent refers to an amount necessary to elicit a desired biological response. As will be appreciated by those of ordinary skill in this art, the absolute amount of a particular agent that is effective may vary depending on such factors as the desired biological endpoint, the agent to be delivered, the target tissue, etc. Those of ordinary skill in the art will further understand that an “effective amount” may be administered in a single dose, or may be achieved by administration of multiple doses. For example, in the case of an agent for the treatment of heart failure, an effective amount may be an amount sufficient to result in clinical improvement of the patient, e.g., increased exercise tolerance/capacity, increased blood pressure, decreased fluid retention, decreased dyspnea, subjective improvement of other symptoms, etc., and/or improved results on a quantitative test of cardiac functioning, e.g., ejection fraction, exercise capacity (e.g., time to exhaustion), etc.

According to certain embodiments of the invention an effective amount results in an improvement in a quantitative measure or index that reflects cardiovascular system functioning or heart failure severity of at least 5%, or preferably at least 10%, at least 20%, or more. For example, an effective amount may increase a measure of exercise capacity by at least 5%, at least 10%, etc., relative to the value in the absence of treatment or when an alternate therapy is administered. An effective amount may increase ejection fraction by at least 5%, at least 10%, etc. According to certain embodiments of the invention, where the value for a quantitative measure or index in a subject suffering from heart failure or a condition or disease associated with heart failure differs from the average value for similar normal subjects (e.g., subjects matched for variables such as age, weight, sex, etc., but not suffering from heart failure or a disease or condition associated with heart failure) or differs from a previous value measured in the same subject when not suffering from heart failure, an effective amount restores the measure or index at least 10%, at least 20%, or at least 50% of the way towards its value as measured in normal, matched subjects or in the same subject when not suffering from heart failure.

Gene: For the purposes of the present invention, the term “gene” has its meaning as understood in the art. In general, a gene is taken to include gene regulatory sequences (e.g., promoters, enhancers, etc.) and/or intron sequences, in addition to coding sequences (open reading frames). It will further be appreciated that definitions of “gene” include references to nucleic acids that do not encode proteins but rather encode functional RNA molecules such as tRNAs. For the purpose of clarity it is noted that, as used in the present application, the term “gene” generally refers to a portion of a nucleic acid that encodes a protein; the term may optionally encompass regulatory sequences. This definition is not intended to exclude application of the term “gene” to non-protein coding expression units but rather to clarify that, in most cases, the term as used in this document refers to a protein coding nucleic acid.

Gene product or expression product: A “gene product” or “expression product” is, in general, an RNA transcribed from the gene (e.g., either pre- or post-processing) or a polypeptide encoded by an RNA transcribed from the gene (e.g., either pre- or post-modification). A compound or agent is said to increase gene expression if application of the compound or agent to a cell or subject results in an increase in either an RNA or polypeptide expression product or both. A compound or agent is said to decrease gene expression if application of the compound or agent to a cell or subject results in a decrease in either an RNA or polypeptide expression product or both.

Hybridize: The term “hybridize”, as used herein, refers to the interaction between two complementary nucleic acid sequences. The phrase “hybridizes under high stringency conditions” describes an interaction that is sufficiently stable that it is maintained under art-recognized high stringency conditions. Guidance for performing hybridization reactions can be found, for example, in Current Protocols in Molecular Biology, John Wiley & Sons, N.Y., 6.3.1-6.3.6, 1989, and more recent updated editions, all of which are incorporated by reference. See also Sambrook, Russell, and Sambrook, Molecular Cloning: A Laboratory Manual, 3^{rd} ed., Cold Spring Harbor Laboratory Press, Cold Spring Harbor, 2001. Aqueous and nonaqueous methods are described in that reference and either can be used. Typically, for nucleic acid sequences over approximately 50-100 nucleotides in length, various levels of stringency are defined, such as low stringency (e.g., 6× sodium chloride/sodium citrate (SSC) at about 45° C., followed by two washes in 0.2×SSC, 0.1% SDS at least at 50° C. (the temperature of the washes can be increased to 55° C. for medium-low stringency conditions)); medium stringency (e.g., 6×SSC at about 45° C., followed by one or more washes in 0.2×SSC, 0.1% SDS at 60° C.); high stringency (e.g., 6×SSC at about 45° C., followed by one or more washes in 0.2×SSC, 0.1% SDS at 65° C.); and very high stringency (e.g., 0.5M sodium phosphate, 0.1% SDS at 65° C., followed by one or more washes at 0.2×SSC, 1% SDS at 65° C.) Hybridization under high stringency conditions only occurs between sequences with a very high degree of complementarity. One of ordinary skill in the art will recognize that the parameters for different degrees of stringency will generally differ based various factors such as the length of the hybridizing sequences, whether they contain RNA or DNA, etc. For example, appropriate temperatures for high, medium, or low stringency hybridization will generally be lower for shorter sequences such as oligonucleotides than for longer sequences.

Isolated: As used herein, “isolated” means 1) separated from at least some of the components with which it is usually associated in nature; 2) prepared or purified by a process that involves the hand of man; and/or 3) not occurring in nature.

Ligand: As used herein, “ligand” means a molecule that specifically binds to a target such as a polypeptide through a mechanism other than an antigen-antibody interaction. The term encompasses, for example, polypeptides, peptides, and small molecules, either naturally occurring or synthesized, including molecules whose structure has been invented by man. Although the term is frequently used in the context of receptors and molecules with which they interact and that typically modulate their activity, the term as used herein applies more generally.

Marker: A “marker” may be any gene or gene product (e.g., protein, peptide, mRNA) that indicates or identifies a particular diseased or physiological state (e.g., carcinoma, normal, dysplasia) or indicates or identifies a particular cell type, tissue type, or origin. The expression or lack of expression of a marker gene may indicate a particular physiological or diseased state of a patient, organ, tissue, or cell. Preferably, the expression or lack of expression may be determined using standard techniques such as Northern blotting, in situ hybridization, RT-PCR, real-time RT-PCR, sequencing, immunochemistry, immunoblotting, oligonucleotide or cDNA microarray or membrane array, protein microarray analysis, mass spectrometry, etc. In certain embodiments of the invention, the level of expression of a marker gene is quantifiable.

Operably linked: As used herein, “operably linked” refers to a relationship between two nucleic acid sequences wherein the expression of one of the nucleic acid sequences is controlled by, regulated by, modulated by, etc., the other nucleic acid sequence. For example, the transcription of a nucleic acid sequence is directed by an operably linked promoter sequence; post-transcriptional processing of a nucleic acid is directed by an operably linked processing sequence; the translation of a nucleic acid sequence is directed by an operably linked translational regulatory sequence; the transport or localization of a nucleic acid or polypeptide is directed by an operably linked transport or localization sequence; and the post-translational processing of a polypeptide is directed by an operably linked processing sequence. Preferably a nucleic acid sequence that is operably linked to a second nucleic acid sequence is covalently linked, either directly or indirectly, to such a sequence, although any effective three-dimensional association is acceptable.

Peptide, polypeptide, or protein: According to the present invention, a “peptide”, “polypeptide”, or “protein” comprises a string of at least three amino acids linked together by peptide bonds. The terms may be used interchangeably although a peptide generally represents a string of between approximately 8 and 30 amino acids. Peptide may refer to an individual peptide or a collection of peptides. Peptides preferably contain only natural amino acids, although non-natural amino acids (i.e., compounds that do not occur in nature but that can be incorporated into a polypeptide chain; see, for example, the web site having URL www.cco.caltech.edu/˜dadgrp/Unnatstruct.gif, which displays structures of non-natural amino acids that have been successfully incorporated into functional ion channels) and/or amino acid analogs as are known in the art may alternatively be employed. Also, one or more of the amino acids in a peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide, but such modifications may confer desirable properties, e.g., enhanced biological activity, on the peptide.

A compound or agent is said to increase expression of a polypeptide if application of the compound or agent to a cell or subject results in an increase in the amount of the polypeptide. A compound or agent is said to decrease expression of a polypeptide if application of the compound or agent to a cell or subject results in a decrease in the amount of the polypeptide.

Polynucleotide or oligonucleotide: “Polynucleotide” or “oligonucleotide” refers to a polymer of nucleotides. Typically, a polynucleotide comprises at least three nucleotides. The polymer may include natural nucleosides (e.g., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

A compound or agent is said to increase expression of a polynucleotide if application of the compound or agent to a cell or subject results in an increase in the amount of the polynucleotide or of a translation product of the polynucleotide or both. A compound or agent is said to decrease expression of a polynucleotide if application of the compound or agent to a cell or subject results in a decrease in the amount of the polynucleotide or of a translation product of the polynucleotide or both.

Prognostic information andpredictive information: As used herein the terms “prognostic information” and “predictive information” are used interchangeably to refer to any information that may be used to foretell any aspect of the course of a disease or condition either in the absence or presence of treatment. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient's disease will respond to a particular therapy (wherein response may be defined in any of a variety of ways). Prognostic and predictive information are included within the broad category of diagnostic information.

Purified: As used herein, “purified” means separated from one or more compounds or entities, e.g., one or more compounds or entities with which it is naturally found. A compound or entity may be partially purified, substantially purified, or pure, where it is pure when it is removed from substantially all other compounds or entities, i.e., is preferably at least about 90%, more preferably at least about 91%, 92%, 93%, 94%, 95%, 96%, 97%, 98%, 99%, or greater than 99% pure. In the context of a preparation of a single nucleic acid molecule, a preparation may be considered substantially pure if the nucleic acid represents a majority of all nucleic acid molecules in the preparation, preferably at least 75%, yet more preferably at least 90%, or greater, as listed above.

Regulatory sequence: The term “regulatory sequence” is used herein to describe a region of nucleic acid sequence that directs, enhances, or inhibits the expression (particularly transcription, but in some cases other events such as splicing or other processing) of sequence(s) with which it is operatively linked. The term includes promoters, enhancers and other transcriptional control elements. In some embodiments of the invention, regulatory sequences may direct constitutive expression of a nucleotide sequence; in other embodiments, regulatory sequences may direct tissue-specific and/or inducible expression. For instance, non-limiting examples of tissue-specific promoters appropriate for use in mammalian cells include lymphoid-specific promoters (see, for example, Calame et al., Adv. Immunol. 43:235, 1988) such as promoters of T cell receptors (see, e.g., Winoto et al., EMBO J 8:729, 1989) and immunoglobulins (see, for example, Banerji et al., Cell 33:729, 1983; Queen et al., Cell 33:741, 1983), and neuron-specific promoters (e.g., the neurofilament promoter; Byrne et al., Proc. Natl. Acad. Sci. USA 86:5473, 1989). Developmentally-regulated promoters are also encompassed, including, for example, the murine hox promoters (Kessel et al., Science 249:374, 1990) and the α-fetoprotein promoter (Campes et al., Genes Dev. 3:537, 1989). In some embodiments of the invention regulatory sequences may direct expression of a nucleotide sequence only in cells that have been infected with an infectious agent. For example, the regulatory sequence may comprise a promoter and/or enhancer such as a virus-specific promoter or enhancer that is recognized by a viral protein, e.g., a viral polymerase, transcription factor, etc.

Sample: As used herein, a “sample” obtained from a subject may include, but is not limited to, any or all of the following: a cell or cells, a portion of tissue, blood, serum, ascites, urine, saliva, amniotic fluid, cerebrospinal fluid, and other body fluids, secretions, or excretions. The sample may be a tissue sample obtained, for example, from skin, muscle, buccal or conjunctival mucosa, placenta, gastrointestinal tract or other organs. A sample of DNA from fetal or embryonic cells or tissue can be obtained by appropriate methods, such as by amniocentesis or chorionic villus sampling. The term “sample” may also refer to any material derived by isolating, purifying, and/or processing a sample obtained directly from a subject. Derived samples may include nucleic acids or proteins extracted from the sample or obtained by subjecting the sample to techniques such as amplification or reverse transcription of mRNA, etc. A derived sample may be, for example, a homogenate, lysate, or extract prepared from a tissue, cells, or other constituent of an organism (e.g., a body fluid).

Small molecule: As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds.

Specific binding: As used herein, the term “specific binding” refers to an interaction between a target molecule (typically a target polypeptide) and a binding molecule such as an antibody or ligand. The interaction is typically dependent upon the presence of a particular structural feature of the target molecule such as an antigenic determinant or epitope recognized by the binding molecule. For example, if an antibody is specific for epitope A, the presence of a polypeptide containing epitope A or the presence of free unlabeled A in a reaction containing both free labeled A and the antibody thereto, will reduce the amount of labeled A that binds to the antibody. It is to be understood that specificity need not be absolute but generally refers to the context in which the binding is performed. For example, it is well known in the art that numerous antibodies cross-react with other epitopes in addition to those present in the target molecule. Such cross-reactivity may be acceptable depending upon the application for which the antibody is to be used. One of ordinary skill in the art will be able to select antibodies having a sufficient degree of specificity to perform appropriately in any given application (e.g., for detection of a target molecule, for therapeutic purposes, etc). It is also to be understood that specificity may be evaluated in the context of additional factors such as the affinity of the binding molecule for the target polypeptide versus the affinity of the binding molecule for other targets, e.g., competitors. If a binding molecule exhibits a high affinity for a target molecule that it is desired to detect and low affinity for nontarget molecules, the antibody will likely be an acceptable reagent for immunodiagnostic purposes. Once the specificity of a binding molecule is established in one or more contexts, it may be employed in other, preferably similar, contexts without necessarily re-evaluating its specificity. In the context of an interaction between an antibody or ligand and a polypeptide, according to certain embodiments of the invention a molecule exhibits specific binding if it binds to the polypeptide at least 5 times as strongly as to other polypeptides present in a cell lysate, e.g., a myocardial cell lysate. According to certain embodiments of the invention a molecule exhibits specific binding if it binds to the polypeptide at least 10 times as strongly as to other polypeptides present in a cell lysate. According to certain embodiments of the invention a molecule exhibits specific binding if it binds to the polypeptide at least 50 times as strongly as to other polypeptides present in a cell lysate. According to certain embodiments of the invention a molecule exhibits specific binding if it binds to the polypeptide at least 100 times as strongly as to other polypeptides present in a cell lysate.

Subject: The term “subject”, as used herein, refers to an individual to whom an agent is to be delivered, e.g., for experimental, diagnostic, and/or therapeutic purposes. Preferred subjects are mammals, including humans. Other preferred mammalian subjects include rats, mice, other rodents, non-human primates, rabbits, sheep, cows, dogs, cats, and other domesticated animals and/or animals of agricultural interest.

Therapeutic target: Certain genes that are differentially expressed in cells, tissues, etc., represent “therapeutic targets”, in that modulating expression of such a gene (e.g., increasing expression, decreasing expression, or altering temporal properties of expression) and/or modulating the activity or level of an expression product of the gene may alter the biochemical or physiological properties of the cell or tissue so as to treat or prevent a disease or clinical condition. For example, in the context of the present invention, modulation of the expression of certain of the differentially expressed genes described herein may treat or prevent heart failure. Modulating the activity of an expression product, e.g., by administering a compound such as a small molecule (e.g., an agonist or antagonist) or antibody that affects the activity, by altering phosphorylation or glycosylation state, may treat or prevent heart failure. Expression products (RNA or polypeptide) of the therapeutic target genes may also be referred to as therapeutic targets.

Certain preferred therapeutic targets include, but are not limited to, genes whose encoded polypeptide comprises an extracellular portion. The prediction of protein orientation with respect to the cell membrane and the existence of transmembrane domains can be performed, for example, using the program TMpred (K. Hofmann & W. Stoffel (1993) TMbase—A database of membrane spanning proteins segments. Biol. Chem. Hoppe-Seyler 347,166) and/or the methods described in Erik L. L. Sonnhammer, Gunnar von Heijne, and Anders Krogh: A hidden Markov model for predicting transmembrane helices in protein sequences. In Proc. of Sixth Int. Conf on Intelligent Systems for Molecular Biology, p 175-182 Ed J. Glasgow, T. Littlejohn, F. Major, R. Lathrop, D. Sankoff, and C. Sensen. Menlo Park, Calif.: AAAI Press, 1998.

Treating: As used herein, “treating” includes reversing, alleviating, inhibiting the progress of, preventing, or reducing the likelihood of the disease, disorder, or condition to which such term applies, or one or more symptoms or manifestations of such disease, disorder or condition.

Vector: The term “vector” is used herein to refer to a nucleic acid molecule capable of mediating entry of, e.g., transferring, transporting, etc., another nucleic acid molecule into a cell. The transferred nucleic acid is generally linked to, e.g., inserted into, the vector nucleic acid molecule. A vector may include sequences that direct autonomous replication, or may include sequences sufficient to allow integration into host cell DNA. Useful vectors include, for example, plasmids (which may comprise sequences derived from viruses), cosmids, and virus vectors. Virus vectors include, e.g., replication defective retroviruses, adenoviruses, adeno-associated viruses, and lentiviruses. As will be evident to one of ordinary skill in the art, virus vectors may include various viral components in addition to nucleic acid(s) that mediate entry of the transferred nucleic acid.

### DETAILED DESCRIPTION OF CERTAIN PREFERRED EMBODIMENTS OF THE INVENTION

I. Overview

Heart failure may be understood as a syndrome involving chronic neuro-endocrine activation precipitated by an inability of the heart to maintain perfusion of body tissues. Although study of individual genes and signaling pathways has provided significant insights into this disease process, such techniques are limited by the interdependence of cellular systems. High throughput techniques such as microarray transcription profiling provide one approach to this problem. Simultaneous examination of the expression levels of thousands of genes or, in some cases, whole genomes have provided a productive strategy in polygenic diseases where complex interplay between gene and environment is prominent (Lloyd-Jones, D. M. et al. Lifetime risk for developing congestive heart failure: the Framingham Heart Study. Circulation 106, 3068-72 (2002)). Patterns of gene expression can be described and promising candidate genes identified for in depth study (Slonim, D. K. From patterns to pathways: gene expression data analysis comes of age. Nat Genet 32 Suppl, 502-8 (2002)).

A few investigators have previously assessed gene expression using microarrays in human heart failure. These investigators compared expression in diseased hearts with expression in normal hearts or compared expression among hearts from patients with different diagnoses (Tan, F. L. et al. The gene expression fingerprint of human heart failure. Proc Natl Acad Sci U S A 99, 11387-92 (2002); Hwang, J. J. et al. Microarray gene expression profiles in dilated and hypertrophic cardiomyopathic end-stage heart failure. Physiol Genomics 10, 31-44 (2002); Yang, J. et al. Decreased SLIM1 expression and increased gelsolin expression in failing human hearts measured by high-density oligonucleotide arrays. Circulation 102, 3046-52 (2000) See also Martin, A. C. & Drubin, D. G. Impact of genome-wide functional analyses on cell biology research. Curr Opin Cell Biol 15, 6-13 (2003)). One drawback of this approach, however, is the small number of unique samples studied compared with large interindividual variation in gene expression. Confounding factors such as age and gender are difficult to control with small sample numbers (Boheler, K. R. et al. Sex- and age-dependent human transcriptome variability: Implications for chronic heart failure. Proc Natl Acad Sci U S A 100, 2754-9 (2003)). Other types of studies have implicated a variety of genes in left ventricular dysfunction (Towbin, J. A. & Bowles, N. E. Molecular genetics of left ventricular dysfunction. Curr Mol Med 1, 81-90 (2001).

In the present invention a different approach has been adopted. Paired samples of left ventricular tissue from heart failure patients were collected both at the time of surgical implantation of a left ventricular assist device (LVAD) and later at the time of cardiac transplantation (Table 1). Such paired samples allow a narrow focus on the changes that result from mechanical offloading of the failing ventricle and minimize the effect of inter-individual variability. Several studies have previously established that functional changes characteristic of heart failure are reversed after offloading (Dipla, K., Mattiello, J. A., Jeevanandam, V., Houser, S. R. & Margulies, K. B. Myocyte recovery after mechanical circulatory support in humans with end-stage heart failure. Circulation 97, 2316-22 (1998); Barbone, A., Oz, M. C., Burkhoff, D. & Holmes, J. W. Normalized diastolic properties after left ventricular assist result from reverse remodeling of chamber geometry. Circulation 104, 1229- 32 (2001)). The inventors have recognized that genes whose expression is either upregulated (i.e., increases) or downregulated (i.e., decreases) after patients with heart failure receive an LVAD are likely to be involved in heart failure and/or in recovery from heart failure. In order to identify such genes, microarray analysis of mRNA expression was performed on the paired samples as described in more detail in Example 1.

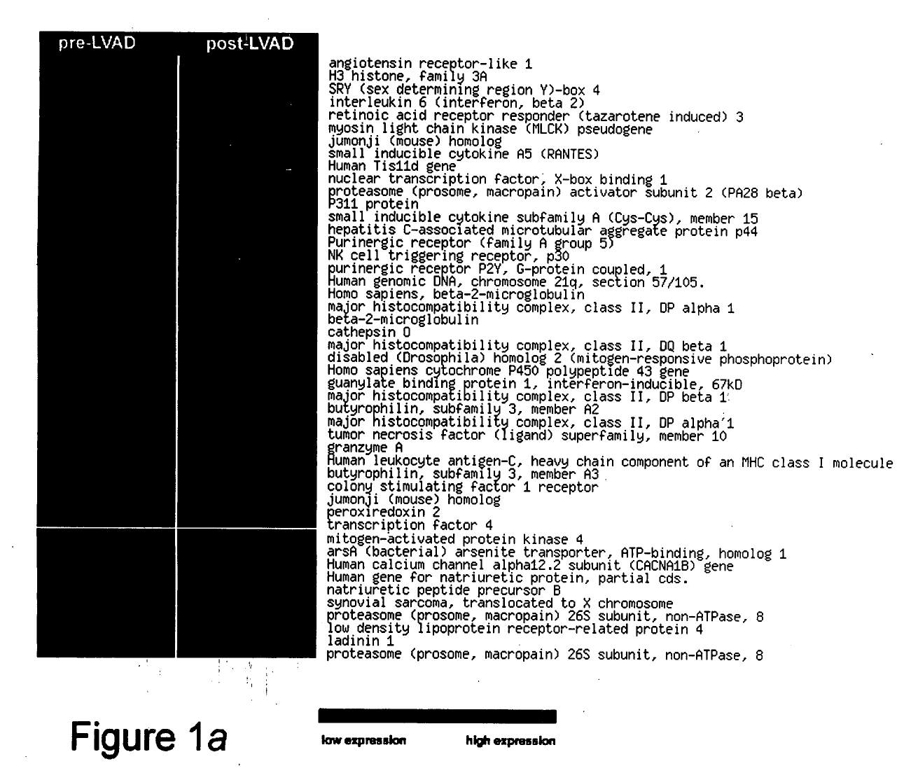

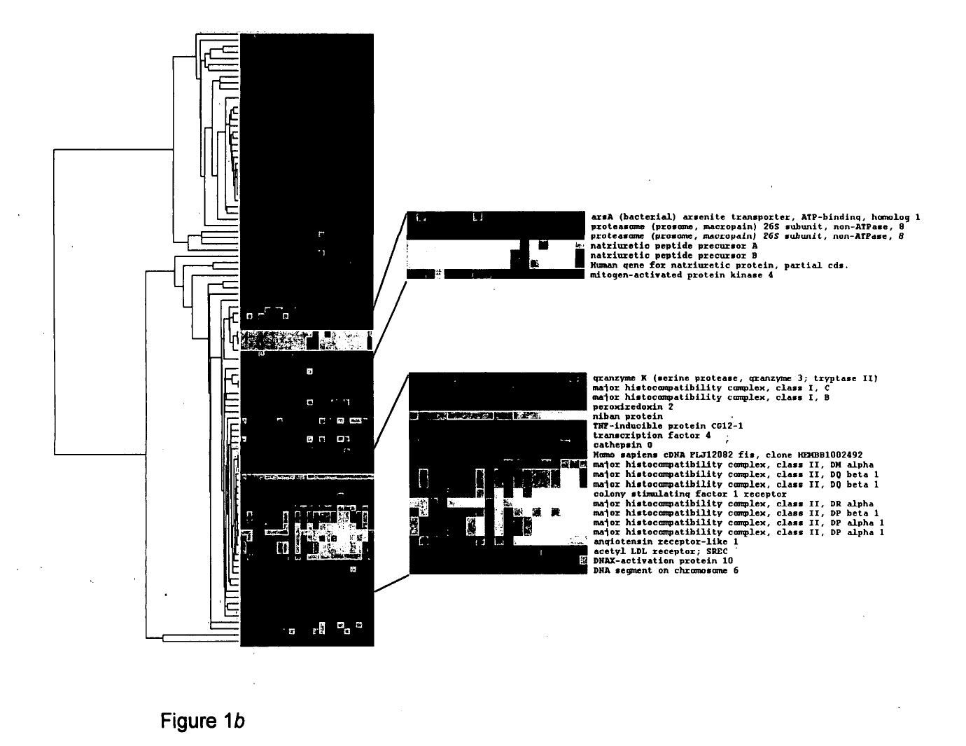

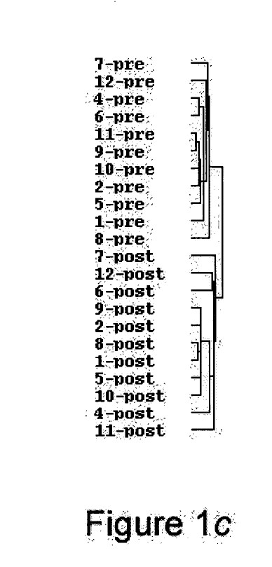

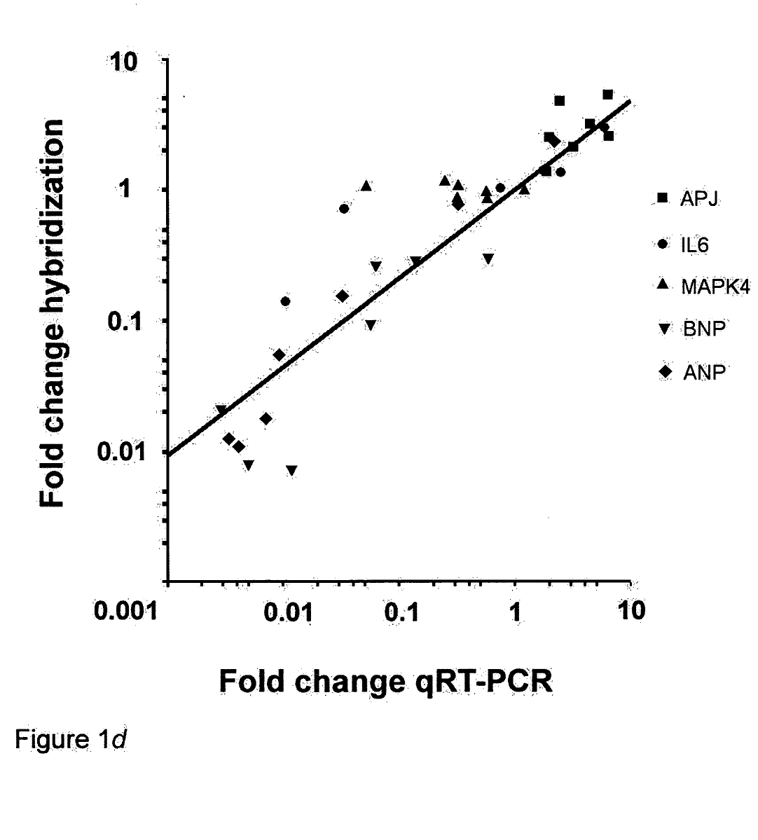

The microarray analysis identified a number of genes with reduced message following offloading achieved by implantation of an LVAD, including several that encode known markers or marker-precursors of heart failure such as natriuretic peptide precursor A (Unigene Hs.75640) and natriuretic peptide precursor B (Unigene Hs.219140). In addition, the natriuretic peptide features clustered together when subjected to average-linkage hierarchical clustering (FIG. 1B). These findings validate the overall approach adopted herein, while the identification of additional genes not known to be significantly upregulated or downregulated in heart failure expands the repertoire of inarkers associated with this condition. Identification of these markers provides a wide variety reagents and methods, as described below. For example, these genes and their expression products, e.g., mRNA and encoded polypeptides, are pharmacological targets for therapies aimed at preventing or treating heart failure or any of its symptoms or manifestations. In addition, identification of genes that are upregulated in heart failure permits the targeting of molecules, including imaging agents and therapeutic agents, e.g., to cardiac tissues, e.g., for purposes including, but not limited to, diagnosis, prognosis, treatment, imaging, or assessment of treatments for conditions associated with heart failure. Measurement of the expression level of the genes newly identified as upregulated or downregulated in heart failure improves diagnosis and prognosis of heart failure. Thus the invention provides diagnostic methods, reagents, and methods for the treatment of heart failure as described below.

It is noted that although the genes identified herein are human genes, the corresponding genes in other mammalian species are also of relevance. In particular, the invention encompasses diagnostic and therapeutic methods for use in non-human mammalian species based on the corresponding genes in such species. While the tissue samples contained cardiac cells of all types, the predominance of myocytes and endothelial cells in the samples indicates that expression data is indicative of the expression state of myocytes and/or endothelial cells. Expression patterns in other cell types present within the heart may be similar.

A. Genes Significantly Upregulated Following LVAD Implantation

The inventors identified a number of genes that are upregulated in cardiac tissue e.g., cardiac myocytes and/or cardiac endothelial cells, in subjects with heart failure following implantation of an LVAD. In other words, these genes were upregulated following mechanical offloading. These genes are listed in Table 2A (see Example 1) by accession number and will be referred to collectively as UIR genes (upregulated in recovery) since their expression increases in association with the recovery from a pathophysiological state of heart failure that occurs upon mechanical offloading. Without being bound by any theory, the inventors propose that such genes are also down-regulated in a state of heart failure, relative to their level in normal subjects.

In particular, genes referred to as angiotensin receptor-like 1 (AGTRL1; Genbank accession number U03642; Unigene number Hs.9305), P311 protein (Genbank accession number NM_{—}004772), guanylate binding protein 1 (Genbank accession number M55542), and tumor necrosis factor (ligand) superfamily member 10 (Genbank accession number U37518) are significantly upregulated following implantation of an LVAD.

AGTRL1, also known as APJ or the APJ receptor, was identified as the gene most significantly and consistently upregulated following mechanical offloading in heart failure among genes represented on the 12,814 feature microarray. APJ is one of a family of seven transmembrane domain receptors first cloned in 1993 (O'Dowd, B. F. et al. A human gene that shows identity with the gene encoding the angiotensin receptor is located on chromosome 11. Gene 136, 355-60 (1993)). Although ‘orphan’ for many years, the endogenous ligand has been isolated and named apelin (Tatemoto, K. et al. Isolation and characterization of a novel endogenous peptide ligand for the human APJ receptor. Biochem Biophys Res Commun 251, 471-6 (1998)).

Apelin and APJ are widely expressed in homogenates from rat and mouse organs (Medhurst, A. D. et al. Pharmacological and immunohistochemical characterization of the APJ receptor and its endogenous ligand apelin. J Neurochem 84, 1162-1172 (2003)) and share identity with angiotensinogen and the angiotensin receptor AT1 respectively. However, angiotensin II does not bind to APJ. Additional characteristics and studies of apelin are described in the following references: Tatemoto, K. et al. The novel peptide apelin lowers blood pressure via a nitric oxide dependent mechanism. Regul Pept 99, 87-92 (2001); Seyedabadi, M., Goodchild, A. K. & Pilowsky, P. M. Site-specific effects of apelin-13 in the rat medulla oblongata on arterial pressure and respiration. Auton Neurosci 101, 32-8 (2002); Szokodi, I. et al. Apelin, the novel endogenous ligand of the orphan receptor APJ, regulates cardiac contractility. Circ Res 91, 434-40 (2002); Katugampola, S. D., Maguire, J. J., Matthewson, S. R. & Davenport, A. P. [(125)1]-(Pyr(1))Apelin-13 is a novel radioligand for localizing the APJ orphan receptor in human and rat tissues with evidence for a vasoconstrictor role in man. Br J Pharmacol 132, 1255-60 (2001); Lee, D. K. et al. Characterization of apelin, the ligand for the APJ receptor. J Neurochem 74, 34-41 (2000); Shah, A. M. Paracrine modulation of heart cell function by endothelial cells. Cardiovasc Res 31, 847-67 (1996); De Falco, M. et al. Apelin expression in normal human tissues. In Vivo 16, 333-6 (2002); Devic, E., Rizzoti, K., Bodin, S., Knibiehler, B. & Audigier, Y. Amino acid sequence and embryonic expression of msr/apj, the mouse homolog of Xenopus X-msr and human APJ. Mech Dev 84, 199-203 (1999)) and below.

B. Genes Significantly Downregulated Following LVAD Implantation

The inventors identified a number of genes that are downregulated in cardiac tissue in subjects with heart failure following mechanical offloading that occurs following implantation of an LVAD. These genes are listed in Table 3 (see Example 1) by accession number and will be referred to collectively as DIR genes (downregulated in recovery) since their expression decreases in association with the recovery from a pathophysiological state of heart failure that occurs upon mechanical offloading. Without wishing to be bound by any theory, the inventors propose that these genes are upregulated in a state of heart failure relative to their expression level in normal subjects. In particular, genes referred to as mitogen activated protein kinase 4 (MAPK4, also called ERK3, ERK4, and p63MAPK, Genbank accession number X59727, Hs.269222) and TEC protein tyrosine kinase (Genbank accession number D29767) are significantly downregulated following implantation of an LVAD. In addition, a splice variant of the regulatory domain (α subunit) of the L-type calcium channel was downregulated after offloading (AF233289), a finding whose significance is underscored by the fact that changes in calcium dynamics are a central component of heart failure pathogenesis. Although the role of myosin light chain kinase pseudogene (AF042089) remains heretofore unknown, myosin light chain kinase itself is a key mediator of sarcomeric organization in cardiac hypertrophy. Myosin light chain 2a (W17098) is a highly conserved and early marker of atrial chamber differentiation in organogenesis but is identified here in the ventricle, suggesting a possible novel role in left ventricular hypertrophy and failure.

C. Polypeptides Encoded by UIR and DIR Genes

Polypeptide expression products of the genes identified in Table 2A are referred to herein as UIR polypeptides. Polypeptide expression products of the genes identified in Table 3 are referred to herein as DIR polypeptides. Such polypeptides include polypeptides comprising the complete amino acid sequence encoded by the corresponding UIR or DIR gene. In addition, in certain embodiments of the invention UIR or DIR polypeptides comprise less than the complete amino acid sequence encoded by the corresponding UIR or DIR gene. For example alternate splicing or post-translational processing may give rise to shorter polypeptides that comprise less than the entire amino acid sequence encoded by the corresponding UIR or DIR gene. In general, such UIR or DIR polypeptides will comprise at least 10 continuous amino acid residues encoded by the corresponding UIR or DIR gene, at least 20 continuous amino acid residues encoded by the corresponding UIR or DIR gene, at least 30 continuous amino acid residues encoded by the corresponding UIR or DIR gene, at least 40 continuous amino acid residues encoded by the corresponding UIR or DIR gene, at least 50 continuous amino acid residues encoded by the corresponding UIR or DIR gene, etc. In various embodiments of the invention a UIR or DIR polypeptide comprises a polypeptide whose sequence comprises at least 10% of the amino acid sequence encoded by the corresponding UIR or DIR gene. In other embodiments of the invention a UIR or DIR polypeptide comprises a polypeptide whose sequence comprises at least 20%, at least 30%, at least 40%, at least 50%, at least 60%, at least 70%, at least 80%, at least 90%, at least 95%, or 100% of the amino acid sequence encoded by the corresponding UIR or DIR gene. In certain embodiments of the invention a UIR or DIR polypeptide consists of the complete polypeptide encoded by the corresponding UIR or DIR gene.

D. Methods of Identifying Genes

The invention provides novel method for identifying genes that are upregulated or downregulated in various physiological states. In particular, the invention provides a method of identifying a diagnostic or therapeutic target gene comprising the steps of: (a) obtaining paired cardiac tissue samples from a subject prior to and following offloading of the heart; (b) assessing expression of at least one gene in the samples; and (c) identifying a gene that is significantly differentially expressed in the two samples. The paired samples may be obtained from a subject with heart failure prior to or in conjunction with implantation of an LVAD (or prior to initiation of any therapy that results in offloading, e.g., mechanical offloading) and then at a later time point, e.g., upon cardiac transplantation, removal of the LVAD, implantation of an artificial heart, etc. Samples may also be obtained using cardiac biopsy, not necessarily in conjunction with surgery. In preferred embodiments of the invention the step of assessing comprises performing a microarray analysis of mRNA expression of a plurality of genes. In certain embodiments at least two data analysis metrics are used to classify genes as differentially regulated (e.g., upregulated or downregulated). For example, as described in Example 1, a significance analysis of microarrays (SAM) score and a rank consistency score can be used. Genes may be identified as significantly differentially regulated by either or both of the scores.

II. APJ and Apelin

As mentioned above, the gene encoding the G-protein coupled receptor known as APJ (also referred to herein as the APJ receptor) was the most significantly upregulated gene following mechanical offloading of the heart. Based on this discovery, the invention provides a variety of different reagents and methods, which are further described elsewhere herein. APJ is a 377 amino acid, 7 transmembrane domain, Gᵢ coupled receptor whose gene is localized on the long arm of chromosome 11 in humans. It was first cloned in 1993 from genomic human DNA using degenerate oligonucleotide primers (O'Dowd B F, Heiber M, Chan A, Heng H H, Tsui L C, Kennedy J L, Shi X, Petronis A, George S R, Nguyen T. A human gene that shows identity with the gene encoding the angiotensin receptor is located on chromosome 11, Gene,136:355-60, 1993) and shares significant homology with angiotensin II receptor type 1. Despite this homology however, angiotensin II does not bind APJ.

The natural ligand for the APJ receptor, apelin, has been isolated from bovine stomach (Tatemoto K, Hosoya M, Habata Y, Fujii R, Kakegawa T, Zou M X, Kawamata Y, Fukusumi S, Hinuma S, Kitada C, Kurokawa T, Onda H, Fujino M. Isolation and characterization of a novel endogenous peptide ligand for the human APJ receptor. Biochem Biophys Res Commun., 251:471-6, 1998). Spanning 1726 base pairs of genomic DNA with 3 exons, the apelin locus is highly conserved between species. Apelin is synthesized as a 77 amino acid preprotein that is cleaved to short peptides of different sizes in different tissues (Kawamata Y, Habata Y, Fukusumi S, Hosoya M, Fujii R, Hinuma S, Nishizawa N, Kitada C, Onda H, Nishimura O, Fujino M. Molecular properties of apelin: tissue distribution and receptor binding. Biochim Biophys Acta, 1538:162-71, 2001). Apelin and APJ are described in and U.S. Pat. Nos. 6,492,234. Such peptides are collectively referred to as apelin and are named according to their length and/or modification state. In particular, apelin-12 (H-Arg-Pro-Arg-Leu-Ser-His-Lys-Gly-Pro-Met-Pro-Phe-OH), apelin-13 (H-Gln-Arg-Pro-Arg-Leu-Ser-His-Lys-Gly-Pro-Met-Pro-Phe-OH), apelin-17, and apelin-36 (H-Leu-Val-Gln-Pro-Arg-Gly-Ser-Arg-Asn-Gly-Pro-Gly-Pro-Trp-Gln-Gly-Gly-Arg-Arg-Lys-Phe-Arg-Arg-Gln -Arg-Pro-Arg-Leu-Ser-His-Lys-Gly-Pro-Met-Pro-Phe-OH) are known to activate APJ. Apelin circulates as pyroglutamylated apelin-13 (Pyr-Arg-Pro-Arg-Leu-Ser-His-Lys-Gly-Pro-Met-Pro-Phe-OH), which is believed to be more stable than the other forms.

According to various embodiments of the invention any peptide obtained by cleavage of the 77 amino acid preproprotein, or the complete preproprotein, may be used in the methods described herein. Preferably the peptide comprises or consists of apelin-12, apelin-13, or pyroglutamylated apelin-13 (PYR-apelin-13). In certain embodiments of the invention a fragment shorter than 12 amino acids is used, e.g., a subfragment of apelin-12, e.g., a fragment consisting of 6, 7, 8, 9, 10, or 11 continuous amino acids of apelin-12. One of ordinary skill in the art will appreciate that various amino acid substitutions, e.g, conservative amino acid substitutions, may be made in the sequence of any of the apelin peptides described herein, without necessarily decreasing its activity. Conservative substitutions (i.e., substitutions with amino acids of comparable chemical characteristics) are particularly preferred. For the purposes of conservative substitution, the non-polar (hydrophobic) amino acids include alanine, leucine, isoleucine, valine, glycine, proline, phenylalanine, tryptophan and methionine. The polar (hydrophilic), neutral amino acids include serine, threonine, cysteine, tyrosine, asparagine, and glutamine. The positively charged (basic) amino acids include arginine, lysine and histidine. The negatively charged (acidic) amino acids include aspartic acid and glutamic acid.

Apelin was originally isolated from bovine stomach extracts by measuring extracellular acidification in a cell line expressing human APJ (Tatemoto K., et al). Apelin appears to exert its effects at least in part by activating Na⁺—H⁺ exchanger (NHE) isoform-1 (which exchanges extracellular Na⁺ for intracellular H⁺) and also by activating Na⁺—Ca⁺⁺ exchanger (NCX) working in reverse mode (Na⁺ out, Ca⁺⁺ in) (Szokodi I, Tavi P, Foldes G, Voutilainen-Myllyla S, Ilves M, Tokola H, Pikkarainen S, Piuhola J, Rysa J, Toth M, Ruskoaho H. Apelin, the novel endogenous ligand of the orphan receptor APJ, regulates cardiac contractility. Circ Res., 91:434-40, 2002). Thus APJ is believed to couple to these proteins and bring about an increase in their functional activity. Thus activation of APJ results in decreased extracellular pH, increased intracellular pH, decreased extracellular Ca⁺⁺ concentration and increased intracellular Ca⁺⁺ concentration.

Apelin has been implicated in cardiovascular function, central autonomic control and fluid homeostasis. The role of apelin in cardiovascular physiology has however been little investigated. Early studies showed a clear decrease in mean arterial pressure following an intravenous bolus injection of apelin in rats (Tatemoto K, Takayama K, Zou M X, Kumaki I, Zhang W, Kumano K, Fujimiya M. The novel peptide apelin lowers blood pressure via a nitric oxide-dependent mechanism. Regul Pept, 99:87-92, 2001; Lee D K, Cheng R, Nguyen T, Fan T, Kariyawasam A P, Liu Y, Osmond D H, George S R, O'Dowd B F. Characterization of apelin, the ligand for the APJ receptor. J Neurochem., 74:34-41, 2000). In addition, APJ knockout mice show an increased vasopressor response to angiotensin II, suggesting a counter-regulatory role in relation to the renin-angiotensin system. However, another group reported that apelin potently contracts isolated human saphenous vein, suggesting the effect of apelin on vascular reactivity remains unclear (Katugampola S D, Maguire J J, Matthewson S R, Davenport A P, [(125)I]-(Pyr(1))Apelin-13 is a novel radioligand for localizing the APJ orphan receptor in human and rat tissues with evidence for a vasoconstrictor role in man, Br J Pharmacol., 132:1255-60, 2001). In relation to myocardial function, Szokodi et al, (refenced above) showed an effect of apelin on the contractility of the isolated rat heart that was both potent (EC50 in the low picomolar range) and efficacious (maximum developed tension was 70% that of isoproterenol). However, despite these significant effects, the role of apelin in vivo, both under normal physiological conditions and in pathological states such as heart failure, has remained unknown. For example, the balance of effects on cardiac loading and intrinsic contractility (ventriculo-vascular coupling) has not been heretofore described.

As detailed in Example 5, the inventors have shown that apelin circulates in plasma and that its plasma levels can be correlated with disease severity in patients with heart failure. This finding provides a basis for diagnostic and prognostic methods based on measuring circulating apelin levels, as further described below.

As described in Example 4, the inventors have also shown that apelin is highly specifically localized to the vasculature in cardiac tissue in the human heart. The localization of apelin in normal human cardiac left ventricle was similar to that in end stage, failing left ventricle. Cardiac vessels stained densely for apelin with negligible staining in myocardial cells. High powered views suggested apelin staining extended to smooth muscle cells also. Despite little staining overall of the myocardium, in the failing heart, apelin was detectable at low levels in the myocardial cells, also suggesting extension of the signaling system in late stage disease. The inventors further showed that both APJ and apelin are expressed in the developing myocardium in mice, with very similar patterns of expression as early as embryonic day 13.5 (Example 6). In the adult mouse heart, immunolocalization of APJ expression was identified in association with both atrial and ventricular myocardial cells (Example 6).

The inventors additionally studied the effects of both acute and chronic apelin administration in normal mice (Example 7). Based on these findings, the inventors propose that administration of apelin is useful in the treatment of heart failure and related conditions and diseases, as further described below.

To further confirm the ability of apelin to improve cardiovascular function in the setting of heart failure, apelin was administered to mice with experimentally induced heart failure, and their exercise capacity was compared with that of controls that did not receive apelin (Example 8). Diminished exercise capacity is one of the major symptoms of heart failure, and functional recovery from cardiovascular disease and damage can be assessed by measuring exercise capacity (e.g., using treadmill exercise tests to induce controlled cardiovascular stress). Exercise capacity is a major prognostic indicator in patients with cardiovascular disease, including heart failure, and also in individuals with no history of cardiovascular disease. Indeed, after adjustment for age, the peak exercise capacity measured in metabolic equivalents (MET) was the strongest predictor of the risk of death among both normal subjects and those with cardiovascular disease in a recent study (Myers J, Prakash M, Froelicher V, Do D, Partington S, Atwood J E., Exercise capacity and mortality among men referred for exercise testing, N Engl J Med., 346(11):793-801, 2002). These findings form the basis of the therapeutic methods involving use of apelin described below.

It is noted that both apelin and APJ are highly conserved across multiple species, and apelin-77 is subject to similar processing, resulting in formation of smaller peptides. In particular, Apelin-12 is 100% identical in human, mouse, and rat. A summary of information on apelin and APJ in human, mouse, and rat is presented in Table 2B.

III. Antibodies that Bind to UIR and DIR Polypeptides

The discovery that UIR and DIR genes are upregulated and downregulated, respectively, upon mechanical offloading in heart failure provides motivation for the production of antibodies that bind to the encoded polypeptides. Such antibodies are usefuil for a variety of purposes including diagnostic, therapeutic, as targeted delivery vehicles or components of such vehicles, for research purposes, etc. The invention provides an antibody that specifically binds to a UIR or DIR polypeptide encoded by a polynucleotide whose sequence comprises the sequence of a polynucleotide whose Genbank accession number is selected from the group of Genbank accession numbers listed in Table 2A or Table 3. In particular, the invention provides an antibody that specifically binds to a UIR or DIR polypeptide encoded by a polynucleotide whose sequence comprises the sequence of any of the polynucleotides whose Genbank accession numbers are listed in Table 2A or 3.

According to certain embodiments of the invention the antibodies are polyclonal antibodies, however in preferred embodiments of the invention they are monoclonal antibodies. Generally applicable methods for producing antibodies are well known in the art and are described extensively in references cited above. It is noted that antibodies can be generated by immunizing animals (or humans) either with a full length polypeptide, a partial polypeptide, fusion protein, or peptide (which may be conjugated with another moiety to enhance immunogenicity). The specificity of the antibody will vary depending upon the particular preparation used to immunize the animal and on whether the antibody is polyclonal or monoclonal. For example, if a peptide is used the resulting antibody will bind only to the antigenic determinant represented by that peptide. It may be desirable to develop and/or select antibodies that specifically bind to particular regions of the polypeptide, e.g., the extracellular domain. Such specificity may be achieved by immunizing the animal with peptides or polypeptide fragments that correspond to that region. Alternately, a panel of monoclonal antibodies can be screened to identify those that specifically bind to the desired region. The invention therefore provides a panel of antibodies for polypeptides encoded by each upregulated or downregulated gene, wherein each member of the panel specifically recognizes a different antigenic determinant present in the polypeptide.

In general, certain preferred antibodies will possess high affinity, e.g., a K_{d} of <200 mM, and preferably, of <100 mM for their target. According to certain embodiments of the invention preferred antibodies do not show significant reactivity with normal tissues other than the heart, e.g., tissues of key importance such as kidney, brain, liver, bone marrow, colon, breast, prostate, thyroid, gall bladder, lung, adrenals, muscle, nerve fibers, pancreas, skin, etc. In the context of reactivity with tissues, the term “significant reactivity”, as used herein, refers to an antibody or antibody fragment, which, when applied to a tissue of interest under conditions suitable for immunohistochemistry, will elicit either no staining or negligible staining, e.g., only a few positive cells scattered among a field of mostly negative cells.

The invention provides various methods of using the antibodies described above. For example, the antibodies may be used to perform immunohistochemical analysis, immunoblotting, ELISA assays, etc., in order to detect the polypeptide to which the antibody specifically binds. In the case of polypeptides that are released into the bloodstream, detection of the polypeptide in a blood sample can provide a diagnostic test for heart failure, as described further below. The antibodies may be used as components of antibody arrays. The antibodies may also be used for imaging studies, as described further below. In addition, the antibodies are useful for delivering attached moieties to target cells in the heart, as a component in a targeted delivery vehicle, and as therapeutic agents.

IV Ligands of UIR and DIR Polypeptides and Methods for their Identification

In another aspect, the invention provides ligands that specifically bind to a UIR or DIR polypeptide. The term “ligand” is intended to encompass any type of molecule other than antibodies as described above. Ligands may be, for example, peptides, non-immunoglobulin polypeptides, nucleic acids, protein nucleic acids (PMAs), aptamers, small molecules, etc. Ligands that specifically bind to any of the UIR or DIR polypeptides described herein may be identified using any of a variety of approaches. For example, ligands may be identified by screening libraries, e.g., small molecule libraries. Naturally occurring or artificial (non-naturally occurring) ligands, particularly peptides or polypeptides, may be identified using a variety of approaches including, but not limited to, those known generically as two- or three-hybrid screens, the first version of which was described in Fields S. and Song O., Nature Jul. 20 1989;340(6230):245-6. Nucleic acid or modified nucleic acid ligands may be identified using, e.g., systematic evolution of ligands by exponential enrichment (SELEX) (Tuerk, C. and Gold., L, “Systematic evolution of ligands by exponential enrichment: RNA ligands to bacteriophage T4 DNA polymerase”, Science 249(4968): 505-10, 1990), or any of a variety of directed evolution techniques that are known in the art. See also Jellinek, D., et al., “Potent 2′-amino-2′-deoxypyrimidine RNA inhibitors of basic fibroblast growth factor”, Biochemistry, 34(36): 11363-72, 1995, describing identification of high-affinity 2′-aminopyrimidine RNA ligands to basic fibroblast growth factor (bFGF). Screens using nucleic acids, peptides, or polypeptides as candidate ligands may utilize nucleic acids, peptides, or polypeptides that incorporate any of a variety of nucleotide analogs, amino acid analogs, etc. Various nucleotide analogs are known in the art, and other modifications of a nucleic acid chain, e.g., in the backbone, can also be used, as described elsewhere herein.

Peptides or polypeptides may incorporate one or more unnatural amino acids (e.g., amino acids that are not naturally found in mammals, or amino acids that are not naturally found in any organism). Such amino acids include, but are not limited to, cyclic amino acids, diamino acids, β-amino acids, homo amino acids, alanine derivatives, phenylalanine boronic acids, proline and pyroglutamine derivatives, etc. Alterations and modifications may include the replacement of an L-amino acid with a D-amino acid, or various modifications including, but not limited to, phosphorylation, carboxylation, alkylation, methylation, etc.

Polypeptides incorporating unnatural amino acids may be produced either entirely artificially or through biological processes, e.g., in living organisms. Use of unnatural amino acids may have a number of advantages. For example, unnatural amino acids may be utilized as building blocks, conformational constraints, molecular scaffolds, or pharmacologically active products. They represent a broad array of diverse structural elements that may be utilized, e.g., for the development of new leads in peptidic and non-peptidic compounds. They may confer desirable features such as enhanced biological activity, proteolytic resistance, etc. See, e.g., Bunin, B. A. et al., Annu. Rep. Med. Chem. 1999, 34, 267; Floyd, C. D. et al., Prog. Med. Chem. 1999, 36, 91; Borman, S. Chem. Eng. News 1999, 77, 33; Brown, R. K. Modern Drug Discovery 1999, 2, 63; and Borman, S. Chem. Eng. News 2000, 78, 53, describing various applications of unnatural amino acids. Once a ligand is identified, modifications such as those described above may be made.

In general, a screen for a ligand that specifically binds to any particular UIR or DIR polypeptide may comprise steps of contacting UIR or DIR polypeptide with a candidate ligand under conditions in which binding can take place; and determining whether binding has occurred. Any appropriate method for detecting binding, many of which are well known in the art, may be used. One of ordinary skill in the art will be able to select an appropriate method taking into consideration, for example, whether the candidate ligand is a small molecule, peptide, nucleic acid, etc. For example, the candidate ligand may be tagged, e.g., with a radioactive molecule. The UIR or DIR polypeptide can then be isolated, e.g., immunoprecipitated from the vessel in which the contacting has taken place, and assayed to determine whether radiolabel has been bound. This approach may be particularly appropriate for small molecules. Binding can be confirmed by any of a number of methods, e.g., plasmon resonance assays. Phage display represents another method for the identification of ligands that specifically bind to UIR or DIR polypeptides. In addition, determination of the three-dimensional structure of a UIR or DIR polypeptide (e.g., using nuclear magnetic resonance, X-ray crystallography, etc.) may facilitate the design of appropriate ligands.

Functional assays may also be used to identify ligands, particularly ligands that behave as agonists or antagonists, activators, or inhibitors of particular polypeptides. For such assays it is necessary that the polypeptide of interest possesses a measurable or detectable functional activity and that such functional activity is increased or decreased upon binding of the ligand. Examples of functional activities of a polypeptide include, e.g., ability to catalyze a chemical reaction either in vitro or in a cell, ability to induce a change of any sort in a biological system, e.g., a change in cellular phenotype, a change in gene transcription, a change in membrane current, a change in intracellular or extracellular pH, a change in the intracellular or extracellular concentration of an ion, etc. when present within a cell or when applied to a cell.

Ligands that bind to UIR or DIR polypeptides have a variety of uses, which are described below. For example, they may serve as components of targeted delivery vehicles and can be used for imaging of the heart. Ligands that modulate the expression and/or activity of a UIR or DIR polypeptide can also be used for therapeutic purposes.

Certain of the methods for identifying ligands may be performed in vitro, e.g., using a UIR or DIR polypeptide or a significantly similar polypeptide or fragment thereof produced using recombinant DNA technology. Certain of the methods may be performed by applying the test compound to a cell that expresses the polypeptide and measuring the expression or activity of the polypeptide, which may involve isolating the polypeptide from the cell and subsequently measuring its amount and/or activity. In certain of the methods the polypeptide may be a variant that includes a tag (e.g., an HA tag, 6×His tag, Flag tag, etc.) which may be used, for example, to facilitate isolation or the variant may be a fusion protein.

In general, an appropriate method for measuring activity of a polypeptide will vary depending on the polypeptide. For example, if the polypeptide has a known biological or enzymatic activity, or is homologous to a polypeptide with a known biological or enzymatic activity, that activity will be measured using any appropriate method known in the art. Thus if the polypeptide is a kinase a kinase assay will be performed. If the molecule is a cytokine, biological assays such as the ability to activate and/or trigger migration of other cell types can be assessed. If the molecule is a growth factor or growth factor receptor, the ability of the polypeptide to cause cell proliferation can be assessed.

Compounds suitable for screening according to the above methods include small molecules, natural products, peptides, nucleic acids, etc. Sources for compounds include natural product extracts, collections of synthetic compounds, and compound libraries generated by combinatorial chemistry. Libraries of compounds are well known in the art. One representative example is known as DIVERSet™, available from ChemBridge Corporation, 16981 Via Tazon, Suite G, San Diego, Calif. 92127. DIVERSet™ contains between 10,000 and 50,000 drug-like, hand-synthesized small molecules. The compounds are pre-selected to form a “universal” library that covers the maximum pharmacophore diversity with the minimum number of compounds and is suitable for either high throughput or lower throughput screening. For descriptions of additional libraries, see, for example, Tan, et al., “Stereoselective Synthesis of Over Two Million Compounds Having Structural Features Both Reminiscent of Natural Products and Compatible with Miniaturized Cell-Based Assays”, Am. Chem Soc. 120, 8565-8566, 1998; Floyd C D, Leblanc C, Whittaker M, Prog Med Chem 36:91-168, 1999. Numerous libraries are commercially available, e.g., from AnalytiCon USA Inc., P.O. Box 5926, Kingwood, Tex. 77325; 3-Dimensional Pharmaceuticals, Inc., 665 Stockton Drive, Suite 104, Exton, Pa. 19341-1151; Tripos, Inc., 1699 Hanley Rd., St. Louis, Mo., 63144-2913, etc. In certain embodiments of the invention the methods are performed in a high-throughput format using techniques that are well known in the art, e.g., in multiwell plates, using robotics for sample preparation and dispensing, etc. Representative examples of various screening methods may be found, for example, in U.S. Pat. Nos. 5,985,829, 5,726,025, 5,972,621, and 6,015,692. The skilled practitioner will readily be able to modify and adapt these methods as appropriate.

Molecular modeling can be used to identify a pharmacophore for a particular target, i.e., the minimum functionality that a molecule must have to possess activity at that target. Such modeling can be based, for example, on a predicted structure for the target (e.g., a two-dimensional or three-dimensional structure). Software programs for identifying such potential lead compounds are known in the art, and once a compound exhibiting activity is identified, standard methods may be employed to refine the structure and thereby identify more effective compounds.

Thus the invention provides a method for screening for a ligand for a UIR or DIR polypeptide comprising steps of: (i) providing a sample comprising a UIR or DIR polypeptide; (ii) contacting the sample with a candidate compound; (iii) determining whether the level of activity of the polypeptide in the presence of the compound is increased or decreased relative to the level of activity of the polypeptide in the absence of the compound; and (iv) identifying the compound as a ligand of the UIR or DIR polypeptide if the level of activity of the UIR or DIR polypeptide is higher or lower in the presence of the compound relative to its level of activity in the absence of the compound. In certain embodiments of the method the sample comprises cells that express the UIR or DIR polypeptide. Identified compounds can be further tested in vitro or in vivo. For example, it may be desirable to include an additional step of (v) administering the compound to an animal suffering from heart failure and evaluating the response. Response can be evaluated in any of a variety of ways, e.g., by assessing clinical features, laboratory data, images, etc.

The invention includes compounds identified using the above methods, e.g., . compounds that increase or decrease one or more activities of a UIR or DIR polypeptide.

In particular, the invention provides methods for identifying ligands of APJ. A description of these methods serves as an illustrative example that may be extended to other receptor-like molecules. One of ordinary skill in the art will be able to develop assays for other types of molecules, given the motivation provided herein to do so. Certain of the methods may be used to identify compounds that increase the level of APJ activity, e.g., by increasing its functional activity, increasing its expression, etc. Such compounds may act in any of a variety of ways including, but not limited to, by binding to APJ. Other methods may be used to identify compounds that inhibit the APJ receptor, i.e., decrease its functional activity. It is noted that small molecule agents targeting a variety of different G-protein coupled receptors have been identified using various screening methods, and numerous therapeutic agents that act via such receptors are known. It is thus expected that screening to identify small molecule activators and/or inhibitors of APJ, while perhaps time-consuming, will be possible without undue experimentation.

According to certain of the inventive screening methods for identifying activators or inhibitors of APJ, the APJ polypeptide is expressed in cells. In general, a wide variety of cells can be used, e.g., Xenopus oocytes, yeast cells, mammalian cells, etc. Numerous different types of mammalian cell lines are suitable, e.g., CHO cells, HEK293 cells, L cells, BHK cells, etc. Primary cells, e.g., cardiac myocytes, can also be used. Candidate compounds are applied to the cells and the intracellular or extracellular pH is detected. An increase in the intracellular pH or a decrease in the extracellular pH (e.g., acidification of the fluid in which the cells are contained) indicates that the candidate compound activates the APJ receptor, leading to an activation of the Na⁺/H⁺ exchanger, which causes an increase in proton flux across the cell membrane, leading to an increase in intracellular pH and a decrease in extracellular pH. pH can be detected using any available means. One convenient method of detecting a change in extracellular pH is to include a pH-sensitive indicator molecule in the fluid containing the cells, e.g., phenol red, bromocresol purple, etc. A property of the molecule such as color, fluorescence, etc., of the dye serves as an indication of the pH. The intracellular pH can be deteted in an analogous manner by loading cells with an appropriate pH-sensitive molecule. Such assays are well known in the art. Use of an imaging system to detect changes in fluorescence, color, etc., facilitates adaptation of the assay to high throughput screening techniques. Devices such as the Cytosensor (Molecular Devices, Sunnyvale, Calif.) may also be used to measure extracellular pH and/or extracellular acidification rate as described in Tatemoto, et al, referenced above.

As mentioned above, activation of the APJ receptor also results in activation of the Na⁺/Ca⁺⁺ exchanger operating in reverse mode, leading to increased intracellular Ca⁺⁺ concentration and a corresponding decrease in extracellular Ca⁺⁺ concentration. Methods for identifying agents that cause increased intracellular Ca⁺⁺ and/or decreased extracellular Ca⁺⁺ are well known in the art and can be used to identify compounds that activate the APJ receptor. For example, membrane Ca⁺⁺ current can be measured. Alternately, flux of Ca⁺⁺ isotopes can be detected. Perhaps the most widely used method of monitoring Ca⁺⁺ is by the use of fluorescent Ca⁺⁺ indicators (Tsien, R. in Methods in Cell Biology, Vol. 30, Taylor, D. L. and Wang, Y-L, Eds., Academic Press (1989) pp. 127-156). These indicators are used to detect Ca⁺⁺ concentration via their fluorescent spectral changes upon Ca⁺⁺ binding. Any of a variety of Ca⁺⁺ indicators, including Fura-2, Indo- 1, Fluo-3 and Rhod-2, and related molecules, can be used to monitor changes in Ca⁺⁺ concentration, which serves to identify agents that activate APJ. An example of the use of Fura 2-AM to measure intracellular Ca⁺⁺ concentration and of the use of ⁴⁵Ca⁺⁺ to measure Ca⁺⁺ in cardiac myocytes is found in Pei, 30 J-M., et al., Am. J. Physiol. Cell Physiol., 285:C1420-1428, 2003.

Compounds that inhibit rather than activate the APJ receptor may be identified using modifications of the assays described above. According to one method, in the absence of an inhibitor, contacting cells that express the APJ receptor with a known activating ligand such as apelin causes an increase in intracellular pH, acidification of the extracellular fluid, and an increase in intracellular Ca⁺⁺. However, in the presence of a compound that inhibits the activity of the APJ receptor (which includes inhibition by blocking access by a ligand) or decreases its expression, the extent to which a given amount of apelin will cause increased acidification of extracellular fluid, increased intracellular pH, increased proton flux, increased intracellular Ca⁺⁺, or decreased extracellular Ca⁺⁺, or increased Ca⁺⁺ flux, will be diminished compared with the effect that would result in the absence of the inhibitory compound. Compounds that exert such an inhibitory, or blocking effect, i.e., compounds that antagonize the effects of known APJ activators, are identified as APJ inhibitors. Analogous methods may be employed to identify inhibitors of other proteins with measurable biochemical activities and known ligands.

Another method that can be of particular use to identify non-peptidic modulators of peptides or their receptors involves a two-tier screening strategy (e.g., of a small molecule library) in which the first screening entails disruption of the interaction between the peptide and a neutralizing monoclonal antibody. Selected compounds are then further characterized by their ability to modulate second messengers in cells containing specific receptors. Binding of the identified small molecules to immobilized peptide (or receptor) may be demonstrated by surface plasmon resonance assays, etc. This strategy has been successfully employed to identify modulators of adrenomedullin and gastrin-releasing peptide (Martinez, A., et al., Identification of Vasoactive Non-Peptidic Positive and Negative Modulators of Adrenomedullin Using a Neutralizing Antibody-Based Screening Strategy, Endocrinology, April 2003).

In general, a wide variety of different compounds can be screened. Numerous libraries of natural products, synthetic molecules, combinatorial libraries, etc., are known in the art, and any of these can be used, as mentioned above. In addition, the assays can be used to test variants of known ligands such as apelin peptides in the case of the APJ receptor. Apelin may be used as a starting material to design ligands with improved affinity, bioavailability, etc. Molecular modeling can be used to identify a pharmacophore for APJ, as described above.

V. Targeted Delivery Vehicles

The invention further provides a variety of delivery vehicles targeted to cardiac cells using antibodies and/or ligands that specifically bind to UIR or DIR polypeptides. In general, delivery vehicles are employed to improve the ability of an active molecule to achieve its desired effect on a cell, tissue, organ, subject, etc., e.g., by increasing the likelihood that the active agent will reach its site of activity. By “delivery vehicle” is meant a natural or artificial substance that is physically associated with an active molecule and provides one or more of the following functions among others: (1) conveys an active molecule within the body; (2) facilitates the uptake of an active molecule by cells, tissues, organs, etc.; (3) increases stability of an active molecule, e.g., increases half-life of the molecule; (4) changes other pharmacokinetic properties of the active molecule from what they would have been in the absence of the delivery vehicle. The active molecule may be associated with the delivery vehicle in any of a number of ways. For example, the active molecule may be bonded to the delivery vehicle (e.g., via covalent or hydrogen bonds). In certain preferred embodiments of the invention the active molecule is dispersed within or encapsulated within the delivery vehicle. By “dispersed within” is meant that individual molecules of the active molecule are intermingled with molecules comprising the material from which the delivery vehicle is made as opposed, for example, to being present as a discrete cluster of molecules.

Preferred targeting agents for use in targeting bind to a UIR or DIR polypeptide or portion thereof that is expressed on the surface of a cardiac cell, e.g., a cardiac myocyte or cardiac endothelial cell. According to the invention antibodies or ligands are incorporated in and/or linked to the delivery vehicle for targeting to cardiac cells. Typically at least the portion of the antibody or ligand that binds to the UIR or DIR polypeptide is present on the surface of the delivery vehicle, while the molecule to be delivered is typically inside. Viral vectors can be engineered to express such binding portions, peptide or polypeptide ligands, etc. Immunoliposomes (antibody-directed liposomes) can also be used. See, e.g., Bendas, G., “Immunoliposomes: a promising approach to targeting cancer therapy”, BioDrugs, 15(4), 215-24, 2001. It is noted that such targeted delivery vehicles may be used for the delivery of a wide variety of agents to cardiac cells. Typically the agent is contained within the liposome's aqueous cavity or is one of the components in its lipid membrane.

The invention further provides a targeting agent, e.g., an antibody or ligand that specifically binds to a UIR or DIR polypeptide, wherein the targeting agent is conjugated to a support. The support can be, for example, a nanosphere, microsphere, or bead. The support can be made out of any of a variety of materials including, but not limited to, agarose, polyacrylamide, nylon, dextran, polyethylene glycol, polysaccharides such as PLA, PLGA or chitosan, other polymers, etc. Such conjugates are useful, for example, for detecting, isolating, or purifying UIR or DIR polypeptides. These conjugates may also serve as delivery vehicles for a UIR or DIR antibody or ligand. According to one approach, the antibodies or ligands of the invention can be conjugated to nanoparticles, which may incorporate moieties such as therapeutic agents or agents useful for imaging, as described, for example, in as described in Li, et al., J. Cell. Biochem. Suppl., 39:65-71, 2002. In addition, the invention provides targeting agents that specifically bind to UIR OR DIR polypeptides, wherein the targeting agents are conjugated to a support, and wherein an additional moiety is conjugated to the support. The additional moiety may be, for example, a therapeutic agent, an imaging agent, a readily detectable marker, an enzyme, etc.

VI. Targeting Agents Linked with a Functional Moiety

In another aspect, the invention provides compositions comprising a targeting agent linked with a functional moiety, wherein the targeting agent specifically binds to a UIR or DIR polypeptide. Targeting agents may be any agent that specifically binds to a UIR or DIR polypeptide. In particular, targeting agents can be antibodies or ligands that specifically bind to a UIR or DIR polypeptide, as described above.

In general, these compositions possesses at least two functions, one of which is specifically binding to a UIR or DIR polypeptide. The antibody may be any of the antibodies described above that bind to UIR or DIR polypeptides. By “functional moiety” is meant any compound, agent, molecule, etc., that possesses an activity or property that alters, enhances, or otherwise changes the ability of the targeting agent to fulfill any particular purpose or that enables the targeting agent to fulfill a new purpose. Such purposes include, but are not limited to, providing diagnostic and/or prognostic information and/or treatment of diseases or conditions associated with heart failure, or imaging the heart.

By “linked” is generally meant covalently bound or, if noncovalently bound, physically associated via intermolecular forces approximately equal in strength to that of covalent bonds. Thus a noncovalent interaction between two molecules that has very slow dissociation kinetics can function as a link. For example, an antibody associated with its cognate antigen is generally considered linked. As another example, reactive derivatives of phospholipids can be used to link the liposomes or cell membranes in which they are incorporated to antibodies or enzymes. Targeting agents, e.g., antibodies or ligands linked with a functional moiety will be referred to herein as conjugates or heteroconjugates. According to certain embodiments of the invention the functional moiety is a compound (e.g., polyethylene glycol) that stabilizes the targeting agent and/or increases its resistance to degradation.

According to certain embodiments of the invention the targeting agent is synthesized using precursors, e.g., amino acids, that contain the functional moiety. For example, an antibody or a polypeptide ligand can be synthesized using amino acid precursors that contain flourine-19 instead of hydrogen at one or more positions, or that contain nitrogen-15 or oxygen-17 instead of the more abundant isotope at one or more positions. As a second example, where the functional moiety is a polypeptide, the composition may be produced as a fusion protein, as described above, wherein one portion of the fusion protein (the antibody or ligand) specifically binds to the UIR or DIR polypeptide and a second portion of the fusion protein consists of or comprises a functional moiety. Alternately, polypeptides may be modified to incorporate a functional moiety. For example, the methods described in Haruta, Y., and Seon, B. K., Proc. Nat. Acad. Sci., 83, 7898-7902 (1986) may be used to iodinate antibodies and other polypeptides. See also Tabata, M., et al., Int. J Cancer, Vol. 82, Issue 5: 737-742, 1999. Functional moieties incorporated into a targeting agent of the invention during synthesis or added to the antibody or ligand subsequently are considered “linked” to the targeting agent.

Functional moieties may be linked to targeting agents such as antibodies by any of a number of methods that are well known in the art. Examples include, but are not limited to, the glutaraldehyde method which couples primarily through the α-amino group and &-amino group, maleimide-sulfhydryl coupling chemistries (e.g., the maleimidobenzoyl-N-hydroxysuccinimide ester (MBS) method), and periodate oxidation methods, which specifically direct the coupling location to the Fc portion of the antibody molecule. In addition, numerous cross-linking agents are known, which may be used to link the targeting agent to the functional moiety.

A wide variety of methods (selected as appropriate taking into consideration the properties and structure of the ligand and functional moiety) may likewise be used to produce the ligand-functional moiety conjugates of the invention. Suitable cross-linking agents include, e.g., carboiimides, N-Hydroxysuccinimidyl-4-azidosalicylic acid (NHS-ASA), dimethyl pimelimidate dihydrochloride (DMP), dimethylsuberimidate (DMS), 3,3′-dithiobispropionimidate (DTBP), etc. According to certain embodiments of the invention the functional moiety is a compound (e.g., polyethylene glycol) that stabilizes the ligand and/or increases its resistance to degradation.

For additional information on conjugation methods and crosslinkers see generally the journal Bioconjugate Chemistry, published by the American Chemical Society, Columbus OH, PO Box 3337, Columbus, Ohio, 43210. This journal reports on advances concerning the covalent attachment of active molecules to biopolymers, surfaces, and other materials. Coverage spans conjugation of antibodies and their fragments, nucleic acids and their analogs, liposomal components, and other biologically active molecules with each other or with any molecular groups that add useful properties. Such molecular groups include small molecules, radioactive elements or compounds, polypeptides, etc. See also “Cross-Linking”, Pierce Chemical Technical Library, available at the Web site having URL www.piercenet.com and originally published in the 1994-95 Pierce Catalog and references cited therein and Wong S S, Chemistry of Protein Conjugation and Crosslinking, CRC Press Publishers, Boca Raton, 1991. The following section presents a number of examples of specific conjugation approaches and cross-linking reagents. However, it is to be understood that the invention is not limited to these methods, and that selection of an appropriate method may require attention to the properties of the particular functional moiety, substrate, or other entity to be linked to the targeting agent.

According to certain embodiments of the invention a bifunctional crosslinking reagent is used to couple a functional moiety with a targeting agent of the invention. In general, bifunctional crosslinking reagents contain two reactive groups, thereby providing a means of covalently linking two target groups. The reactive groups in a chemical crosslinking reagent typically belong to the classes of functional groups—including succinimidyl esters, maleimides, and iodoacetamides. Bifunctional chelating agents may also be used. For example, a targeting agent of the invention may be coupled with a chelating agent, which may be used to chelate a functional moiety such as a metal. Bifunctional chelating agents may be used to couple more than one functional moiety to a targeting agent of the invention. For example, according to certain embodiments of the invention one or more of the functional moieties is useful for imaging and/or one or more of the functional moieties is useful for therapy. Appropriate chelating agents for use with the antibodies or ligands of the invention include polyaminocarboxylates, e.g., DTPA, macrocyclic polyaminocarboxylates such as 1,4,7,10-tetraazacyclododecane N,N′,N″,N′″-tetraacetic acid (DOTA), etc. See Lever, S., J. Cell. Biochem. Suppl., 39:60-64, 2002, and references therein.

The most common schemes for forming a heteroconjugate involve the indirect coupling of an amine group on one biomolecule to a thiol group on a second biomolecule, usually by a two- or three-step reaction sequence. The high reactivity of thiols and their relative rarity in most biomolecules make thiol groups good targets for controlled chemical crosslinking. If neither molecule contains a thiol group, then one or more can be introduced using one of several thiolation methods. The thiol-containing biomolecule may then be reacted with an amine-containing biomolecule using a heterobifunctional crosslinking reagent, e.g., a reagent containing both a succinimidyl ester and either a maleimide or an iodoacetamide. Amine-carboxylic acid and thiol-carboxylic acid crosslinking may also be used. For example, 1-Ethyl-3-(3-dimethylaminopropyl)carbodiimide (EDAC) can react with biomolecules to form “zero-length” crosslinks, usually within a molecule or between subunits of a protein complex. In this chemistry, the crosslinking reagent is not incorporated into the final product. The water-soluble carbodiimide EDAC crosslinks a specific amine and carboxylic acid between subunits of allophycocyanin, thereby stabilizing its assembly. See, e.g., Yeh S W, et al., “Fluorescence properties of allophycocyanin and a crosslinked allophycocyanin trimer.”, Cytometry 8, 91-95 (1987).

Several methods are available for introducing thiols into biomolecules, including the reduction of intrinsic disulfides, as well as the conversion of amine, aldehyde or carboxylic acid groups to thiol groups. Disulfide crosslinks of cystines in proteins can be reduced to cysteine residues by dithiothreitol (DTT), tris-(2-carboxyethyl)phosphine (TCEP), or or tris-(2-cyanoethyl)phosphine. Amines can be indirectly thiolated by reaction with succinimidyl 3-(2-pyridyldithio)propionate (SPDP) followed by reduction of the 3-(2-pyridyldithio)propionyl conjugate with DTT or TCEP. Amines can be indirectly thiolated by reaction with succinimidyl acetylthioacetate followed by removal of the acetyl group with 50 mM hydroxylamine or hydrazine at near-neutral pH. Tryptophan residues in thiol-free proteins can be oxidized to mercaptotryptophan residues, which can then be modified by iodoacetamides or maleimides

Reagents used to crosslink liposomes, cell membranes and potentially other lipid assemblies to biomolecules typically comprise a phospholipid derivative to anchor one end of the crosslink in the lipid layer and a reactive group at the other end to attach the membrane assembly to the target biomolecule.

For purpose of covalently linking active molecules (e.g., therapeutic agents) to targeting agents, it may be preferred to select methods that result in a conjugate wherein the targeting agent is separable from the toxin to allow the toxin to enter the cell. Thiol-cleavable, disulfide-containing conjugates may be employed for this purpose. Cells are able to break the disulfide bond in the cross-linker, which permits release of the toxin within the target cell. Examples of suitable cross-linkers include 2-Iminothiolane (Traut's reagent), N-succinimidyl 3-(2-pyridyldithio)propionate (SPDP), etc. In addition, it is generally preferable to select methods that do not significantly impair the ability of the targeting agent to specifically bind to its target and do not significantly impair the ability of the functional moiety to perform its intended function. One of ordinary skill in the art will be able to test the conjugate to determine whether the targeting agent retains binding ability and/or whether the functional moiety retains its function.

According to certain embodiments of the invention the functional moiety is released from the targeting agent upon uptake into the cell. For example, the functional moiety may be attached to the targeting agent via a linker or spacer that is cleaved by an intracellular enzyme. According to certain embodiments of the invention the functional moiety is an antisense molecule, ribozyme, siRNA, or shRNA which may be targeted to any transcript present in a cardiac cell, e.g., a cardiac myocyte or endothelial cell present within a cardiac vessel. In general, the antibodies and ligands of the invention that specifically bind to UIR or DIR polypeptides may be used as described in Allen, T., Nature Reviews Cancer, Vol. 2, pp. 750-765, 2002, and references therein.

According to certain embodiments of the invention the functional moiety is one that causes, either directly or indirectly, a change in the physiological (i.e., functional) and/or biochemical state of a cell with which it comes into contact. In general, a change in the physiological state of a cell will involve multiple biochemical changes. By “directly causing” is meant that the functional moiety either causes the change itself or by interacting with one or more cellular or extracellular constituents (e.g., nucleic acid, protein, lipid, carbohydrate, etc.) not introduced or induced by the hand of man. The category of direct causation includes instances in which the functional moiety initiates a “pathway”, e.g., in which the functional moiety interacts with one or more constituents, which causes a change in the interaction(s) of this constituent with other constituents, ultimately leading to the alteration in physiological or biochemical state of the cell. By “indirectly causing” is meant either (i) that the functional moiety itself does not cause the change but must be converted into an active form (e.g., by a cellular enzyme) in order to cause the change; or (ii) that the functional moiety itself does not cause the change but instead acts on a second agent that causes the change, which second agent is also introduced to or induced in the cell, its surface, or vicinity by the hand of man.

Various examples of changes in physiological or biological state include, but are not limited to, increases or decreases in gene expression (e.g., increases or decreases in transcription, translation, and/or mRNA or protein turnover), alterations in subcellular localization or secretion of a cellular constituent, alteration in cell viability or growth rate, alteration in differentiation state, etc. According to certain embodiments of the invention the functional moiety is a growth stimulatory or inhibitory agent. For example, the functional moiety may comprise or encode a growth factor, a growth factor receptor, or an agonist or antagonist of a growth factor receptor, wherein the growth factor, growth factor receptor, growth factor receptor agonist, or growth factor receptor antagonist stimulates or inhibits growth or division of cardiac cells, and wherein presence of the growth factor receptor in or on the surface of cardiac cells, e.g., cardiac myocytes or cardiac endothelial cells may either stimulate or inhibit its growth or division depending at least in part on the presence of agonists or antagonists.

Whether any particular functional moiety stimulates or inhibits growth and/or division of cardiac cells may readily be tested either using in vitro tissue culture systems in which cardiac cells are contacted with the functional moiety is and their growth and/or division is then measured, or in vivo, in either animals or humans. In the latter case, the ability of the moiety to stimulate or inhibit growth and/or division of cardiac cells may be assessed using, for example, various imaging techniques (see below), or by taking samples of cardiac tissue and assessing its proliferative state (e.g., by determining the mitotic index, measuring expression or activity of proteins associated with cell division, etc.).

According to certain embodiments of the invention the functional moiety is a nucleic acid, which may serve as a template for a transcript to be expressed in the cell. The transcript may encode a polypeptide to be expressed within the cell or may act as a ribozyme, antisense molecule, siRNA, shRNA (or precursor thereof), any of which may reduce or inhibit expression of a target transcript, e.g., by cleaving the transcript (in the case of ribozymes), causing degradation of the transcript, and/or inhibiting its translation. It will be appreciated that the effect of a ribozyme, antisense molecule, siRNA, or shRNA will depend, in general, upon the particular target transcript. In certain embodiments of the invention the ribozyme, antisense molecule, siRNA, or shRNA is toxic to the cell.

VII. Reagents and Methods for Detection and Imaging of Cardiovascular Tissue

As described above, the invention provides a composition comprising a targeting agent linked to a functional moiety, wherein the targeting agent specifically binds to a UIR or DIR polypeptide. According to certain embodiments of the invention the functional moiety is a readily detectable moiety. In general, a readily detectable moiety has a property such as fluorescence, chemiluminescence, radioactivity, color, magnetic or paramagnetic properties, etc., which property renders it detectable by instruments that detect fluorescence, chemiluminescence, radioactivity, color, or magnetic resonance, etc. Alternately, a readily detectable moiety may comprise or encode an enzyme that acts on a substrate to produce a readily detectable compound. According to certain embodiments of the invention the readily detectable moiety is one that, when present at a target site subsequent to administration of the inventive composition to a subject, can be detected from outside the body. In certain preferred embodiments of the invention the readily detectable moiety can be detected non-invasively.

A variety of different moieties suitable for imaging (e.g., moieties suitable for detection by X-ray, fluoroscopy, computed tomography, magnetic resonance imaging, positron emission tomography, gamma tomography, electron spin resonance imaging, optical or fluorescence microscopy, etc.) can be used. Such agents are referred to herin as “imaging agents”. Imaging agents include, but are not limited to, radioactive, paramagnetic, or supraparamagnetic atoms (or molecules containing them). Suitable radioactive atoms include technetium-99m, thallium-211, iodine-133; atoms with magnetic moments such as iodine-123, iodine-131, indium-111, fluorine-19, carbon-13, nitrogen-15, oxygen-17, gadolinium, manganese, or iron. Other suitable atoms include rhenium-186 and rhenium-188. Useful paramagnetic ions include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), europium, and erbium (III), with gadolinium being particularly preferred. Gd-chelates, e.g., DTPA chelates, may be used. For example, the water soluble Gd(DTPA)²-chelate, is one of the most widely used contrast enhancement agents in experimental and clinical imaging research. The DTPA chelating ligand may be modified, e.g., by appending one or more functional groups preferably to the ethylene diamine backbone. Ions useful in other contexts, such as X-ray imaging, include but are not limited to lanthanum (III), gold (III), lead (II), and bismuth (III). Additional moieties useful for imaging include gallium-67, copper-67, yttrium-90, and astatine-211. Moieties useful for optical or fluorescent detection include fluorescein and rhodamine and their derivatives. Agents that induce both optical contrast and photosensitivity include derivatives of the phorphyrins, anthraquinones, anthrapyrazoles, perylenequinones, xanthenes, cyanines, acridines, phenoxazines and phenothiazines (Diwu, Z. J. and Lown, J. W., Pharmacology and Theraeutics 63: 1-35, 1994; Grossweiner, L. I., American Chemical Society Symposium Series 559: 255-265, 1994). Further information regarding methods and applications of molecular imaging in contexts including basic research, diagnosis, therapeutic monitoring, drug development, etc., may be found in articles appearing in the Journal of Cellular Biochemistry, Volume 87, Issue S39 (Supplement), 2002.

The readily detectable moiety may be linked to the targeting agent using various methods as described above. It is noted that many of these moieties may also be useful for therapeutic applications. See, e.g., U.S. Pat. Nos. 5,021,236 and 4,472,509, for various diagnostic agents known in the art to be useful for imaging purposes and methods for their attachment to antibodies. See also discussion above describing coupling of antibodies and ligands of the invention with functional moieties.

According to certain embodiments of the invention the functional moiety is able to bind to an additional moiety, which may impart additional functions. For example, the functional moiety may be a bispecific antibody, one portion of which binds to the UIR OR DIR polypeptide and another portion of which binds to a second molecule, e.g., another functional moiety. Alternately, a second molecule may be linked covalently to the functional moiety.

Accordingly, the invention provides a method of imaging cardiovascular tissue in a sample or subject, comprising steps of: (i) administering to the sample or subject an effective amount of a targeting agent that specifically binds to a UIR or DIR polypeptide, wherein the targeting agent is linked to a functional moiety that enhances detectability of cardiac cells by an imaging procedure; and (ii) subjecting the sample or subject to the imaging procedure. The targeting agent may be, for example, an antibody or ligand that specifically binds to the polypeptide. The methods are useful for imaging the heart for any of a wide variety of purposes. In general, the level of expression of the UIR or DIR polypeptide will be reflected in a characteristic of the image such as intensity. The level of expression can be useful in diagnosing disease (e.g., heart failure and related conditions), assessing disease severity, and/or monitoring the course of the disease or response to treatment. Appropriate imaging procedures include, but are not limited to, X-ray, fluoroscopy, computed tomography, magnetic resonance imaging, positron emission tomography and variants thereof such as SPECT or CT-PET, gamma tomography, electron spin resonance imaging, optical or fluorescence microscopy, etc.

In the case of certain of the UIR and DIR genes identified herein, this work provides the first evidence that these genes are expressed in cardiac tissue. Imaging the expression of these genes will be useful for purposes unrelated to assessing risk or severity of heart failure, response to treatment for heart failure, etc. For example, the fact that these genes are expressed in cardiac tissue indicates that detecting their expression, e.g., by means of imaging, will allow visualization of cardiac tissues for purposes such assessing cardiac structure, assessing the functional capacity of the heart, etc.

VIII. Reagents and Methods for Modulating Expression and/or Activity of UIR and DIR Polynucleotides and Polypeptides

Since the UIR and DIR genes are potential therapeutic targets for heart failure, it is desirable to be able to modulate their expression and/or activity, both for therapeutic and other purposes. The invention therefore provides a variety of methods for altering expression and/or functional activity of a UIR or DIR gene, which are further described below. The invention encompasses methods for screening compounds for preventing or treating heart failure or a disease or clinical condition associated with heart failure by assaying the ability of the compounds to modulate the expression of the DIR or UIR genes disclosed herein or activity of the protein products of these genes. Appropriate screening methods include, but are not limited to, assays for identifying compounds and other substances that interact with (e.g., bind to) the target gene protein products.

A. Methods for Reducing Gene Expression

1. Antisense Nucleic Acids and Methods of Use

Antisense nucleic acids are generally single-stranded nucleic acids (DNA, RNA, modified DNA, modified RNA, or peptide nucleic acids) complementary to a portion of a target nucleic acid (e.g., an mRNA transcript) and therefore able to bind to the target to form a duplex. Typically they are oligonucleotides that range from 15 to 35 nucleotides in length but may range from 10 up to approximately 50 nucleotides in length. Binding typically reduces or inhibits the function of the target nucleic acid. For example, antisense oligonucleotides may block transcription when bound to genomic DNA, inhibit translation when bound to mRNA, and/or lead to degradation of the nucleic acid. Reduction in expression of a UIR or DIR polypeptide may be achieved by the administration of an antisense nucleic acid or peptide nucleic acid (PNA) comprising sequences complementary to those of the mRNA that encodes the polypeptide. Antisense technology and its applications are well known in the art and are described in Phillips, M. I. (ed.) Antisense Technology, Methods Enzymol., Volumes 313 and 314, Academic Press, San Diego, 2000, and references mentioned therein. See also Crooke, S. (ed.) “Antisense Drug Technology: Principles, Strategies, and Applications” (1^{st} ed), Marcel Dekker; ISBN: 0824705661; 1st edition (2001) and references therein.

Peptide nucleic acids (PNA) are analogs of DNA in which the backbone is a pseudopeptide rather than a sugar. PNAs mimic the behavior of DNA and bind to complementary nucleic acid strands. The neutral backbone of a PNA can result in stronger binding and greater specificity than normally achieved using DNA or RNA. Binding typically reduces or inhibits the function of the target nucleic acid. Peptide nucleic acids and their use are described in Nielsen, P. E. and Egholm, M., (eds.) “Peptide Nucleic Acids: Protocols and Applications” (First Edition), Horizon Scientific Press, 1999.

According to various embodiments of the invention the antisense oligonucleotides have a variety of lengths. For example, they may comprise between 8 and 60 contiguous nucleotides complementary to a UIR or DIR mRNA, between 10 and 60 contiguous nucleotides complementary to a UIR or DIR mRNA, or between 12 and 60 contiguous nucleotides complementary to a UIR or DIR mRNA. According to certain embodiments of the invention a UIR or DIR antisense olignucleotide need not be perfectly complementary to the corresponding mRNA but may have up to 1 or 2 mismatches per 10 nucleotides when hybridized to the corresponding mRNA.

The invention further encompasses a method of inhibiting expression of a UIR or DIR polypeptide in a cell or a subject comprising delivering a UIR or DIR antisense oligonucleotide to the cell or subject or expressing such an antisense oligonucleotide within a cell or cells of the subject. In addition, the invention provides a method of treating a condition associated with heart failure comprising steps of (i) providing a subject in need of treatment for a condition associated with heart failure; and (ii) administering a pharmaceutical composition comprising an effective amount of a UIR or DIR antisense oligonucleotide to the subject, thereby alleviating one or more symptoms of heart failure in the subject.

2. UIR or DIR Ribozymes and Methods of Use

Ribozymes (catalytic RNA molecules that are capable of cleaving other RNA molecules) represent another approach to reducing gene expression. Such ribozymes can be designed to cleave specific mRNAs corresponding to a gene of interest. Their use is described in U.S. Pat. No. 5,972,621, and references therein. Extensive discussion of ribozyme technology and its uses is found in Rossi, J. J., and Duarte, L. C., Intracellular Ribozyme Applications: Principles and Protocols, Horizon Scientific Press, 1999.

The invention provides a ribozyme designed to cleave UIR or DIR mRNA. The invention further encompasses a method of inhibiting expression of a UIR or DIR polypeptide in a cell or subject comprising delivering a ribozyme designed to cleave UIR or DIR mRNA to the cell or subject or expressing such a ribozyme within a cell or cells of the subject. In addition, the invention provides a method of treating a condition associated with heart failure comprising steps of (i) providing a subject in need of treatment for a condition associated with heart failure; and (ii) administering a pharmaceutical composition comprising an effective amount of a ribozyme designed to cleave UIR or DIR mRNA to the subject, thereby alleviating the condition.

3. Reagents for Reducing Expression by RNA Interference and Methods of Use

RNA interference (RNAi) is a mechanism of post-transcriptional gene silencing mediated by double-stranded RNA (dsRNA), which is distinct from the antisense and ribozyme-based approaches described above. dsRNA molecules are believed to direct sequence-specific degradation of mRNA that contain regions complementary to one strand (the antisense strand) of the dsRNA in cells of various types after first undergoing processing by an RNase III-like enzyme called DICER (Bernstein et al., Nature 409:363, 2001) into smaller dsRNA molecules. These molecules comprise two 21 nt strands, each of which has a 5′ phosphate group and a 3′ hydroxyl, and includes a 19 nt region precisely complementary with the other strand, so that there is a 19 nt duplex region flanked by 2 nt-3′ overhangs. RNAi is thus mediated by short interfering RNAs (siRNA), which typically comprise a double-stranded region approximately 19 nucleotides in length with 1-2 nucleotide 3′ overhangs on each strand, resulting in a total length of between approximately 21 and 23 nucleotides. In mammalian cells, dsRNA longer than approximately 30 nucleotides typically induces nonspecific mRNA degradation via the interferon response. However, the presence of siRNA in mammalian cells, rather than inducing the interferon response, results in sequence-specific gene silencing.

RNAi can also be achieved using molecules referred to as short hairpin RNAs (shRNA), which are single RNA molecules comprising at least two complementary portions capable of self-hybridizing to form a duplex structure sufficiently long to mediate RNAi (typically at least 19 base pairs in length), and a loop, typically between approximately 1 and 10 nucleotides in length and more commonly between 4 and 8 nucleotides in length that connects the two nucleotides that form the last nucleotide pair at one end of the duplex structure. As described further below, shRNAs are thought to be processed into siRNAs by the conserved cellular RNAi machinery. Thus shRNAs are precursors of siRNAs and are similarly capable of inhibiting expression of a target transcript.

siRNAs and shRNAs have been shown to downregulate gene expression when transferred into mammalian cells by such methods as transfection, electroporation, or microinjection, or when expressed in cells via any of a variety of plasmid-based approaches. RNA interference using siRNA and/or shRNA is reviewed in, e.g., Tuschl, T., Nat. Biotechnol., 20: 446-448, May 2002. See also Yu, J., et al., Proc. Natl. Acad. Sci., 99(9), 6047-6052 (2002); Sui, G., et al., Proc. Natl. Acad. Sci., 99(8), 5515-5520 (2002); Paddison, P., et al., Genes and Dev., 16, 948-958 (2002); Brummelkamp, T., et al., Science, 296, 550-553 (2002); Miyagashi, M. and Taira, K., Nat. Biotech., 20, 497-500 (2002); Paul, C., et al., Nat. Biotech., 20, 505-508 (2002). A number of variations in structure, length, number of mismatches, size of loop, identity of nucleotides in overhangs, etc., are consistent with effective RNAi-mediated gene silencing. For example, one or more mismatches between the target mRNA and the complementary portion of the siRNA or shRNA may still be compatible with effective silencing.

It is thought that intracellular processing (e.g., by DICER) of a variety of different precursors results in production of RNAs of various kinds that are capable of effectively mediating gene silencing. For example, in addition to the siRNA and shRNA structures described above, DICER can process ˜70 nucleotide hairpin precursors with imperfect duplex structures, i.e., duplexes that are interrupted by one or more mismatches, bulges, or inner loops within the stem of the hairpin into single-stranded RNAs called microRNAs (miRNA) that are believed to hybridize within the 3′ UTR of a target mRNA and repress translation. See, e.g., Lagos-Quintana, M. et al., Science, 294, 853-858, 2001; Pasquinelli, A., Trends in Genetics, 18(4), 171-173, 2002, and references in the foregoing two articles for discussion of miRNAs and their mechanisms of silencing.

Accordingly, the invention provides siRNA and shRNA compositions targeted to mRNA encoding any of the UIR or DIR polypeptides. The term “UIR or DIR siRNA” includes any siRNA or shRNA (or precursors thereof) targeted to a UIR or DIR mRNA transcript. An siRNA, shRNA, or miRNA is considered “targeted” to an mRNA if (i) the stability of the target transcript is reduced in the presence of the siRNA as compared with its absence (or, for RNAs that act by inhibiting translation, translation of the target transcript is reduced in the presence of the RNA as compared with its absence); and/or (ii) the duplex portion of the siRNA or shRNA shows at least about 80%, preferably at least about 90%, more preferably at least about 91%, 92%, 93%, 94%, 95%, 96%, 97%, 98%, 99%, or 100% precise sequence complementarity with the target transcript for a stretch of at least about 17, more preferably at least about 18 or 19 to about 21-23 nucleotides; and/or (iii) the siRNA hybridizes to the target transcript under stringent conditions (selected taking into account the length of the siRNA). Typically at least two, and generally all three of the criteria will be met.

The invention encompasses a method of inhibiting expression of a UIR or DIR gene in a cell or subject comprising delivering an siRNA or shRNA targeted to UIR or DIR MRNA to the cell or subject. In addition, the invention provides a method of treating a condition associated with heart failure comprising steps of (i) providing a subject in need of treatment for heart failure or a disease or condition associated with heart failure; and (ii) administering a pharmaceutical composition comprising an effective amount of an siRNA or shRNA targeted to UIR or DIR mRNA to the subject, thereby alleviating the condition.

As mentioned above, siRNAs and shRNAs have been shown to effectively reduce gene expression when expressed intracellularly, e.g., by delivering vectors such as plasmids, viral vectors such as adenoviral, retroviral or lentiviral vectors, or viruses to cells. Such vectors, referred to herein as RNAi-inducing vectors, are vectors whose presence within a cell results in transcription of one or more RNAs that self-hybridize or hybridize to each other to form an shRNA or siRNA. In general, the vector comprises a nucleic acid operably linked to expression signal(s) so that one or more RNA molecules that hybridize or self-hybridize to form an siRNA or shRNA are transcribed when the vector is present within a cell. Thus the vector provides a template for intracellular synthesis of the RNA or RNAs or precursors thereof. The vector will thus contain a sequence or sequences whose transcription results in synthesis of two complementary RNA strands having the properties of siRNA strands described above, or a sequence whose transcription results in synthesis of a single RNA molecule containing two complementary portions separated by an intervening portion that forms a loop when the two complementary portions hybridize to one another.

Selection of appropriate siRNA and shRNA sequences can be performed according to guidelines well known in the art, e.g., taking factors such as desirable GC content into consideration. See, e.g., Ambion Technical Bulletion #506, available at the web site having URL www.ambion.com/techlib/tb/tb_{—}506.html. Following these guidelines approximately half of the selected siRNAs effectively silence the corresponding gene, indicating that by selecting about 5 siRNAs it will almost always be possible to identify an effective sequence. A number of computer programs that aid in the selection of effective siRNA/shRNA sequences are known in the art, which yield even higher percentages of effective siRNAs. See, e.g., Cui, W., et al., “OptiRNai, a Web-based Program to Select siRNA Sequences”, Proceedings of the IEEE Computer Society Conference on Bioinformatics, p. 433, 2003. Pre-designed siRNAs targeting over 95% of the mouse or human genome are commercially available, e.g, from Ambion and/or Cenix Biosciences. See web site having URL www.ambion.com/techlib/tn/104/5.html. As is known in the art, siRNAs and shRNAs can be delivered using a variety of delivery agents that increase their potency.

4. Synthesis, Delivery Methods and Modifications

Antisense nucleic acids, ribozymes, siRNAs, or shRNAs can be delivered to cells by standard techniques such as microinjection, electroporation, or transfection. Antisense nucleic acids, ribozymes, siRNAs, or shRNAs can be formulated as pharmaceutical compositions and delivered to a subject using a variety of approaches, as described further below. According to certain embodiments of the invention the delivery of antisense, ribozyme, siRNA, or shRNA molecules is accomplished via a gene therapy approach in which vectors (e.g., viral vectors such as retroviral, lentiviral, or adenoviral vectors, etc.) are delivered to a cell or subject, or cells directing expression of the molecules (e.g., cells into which a vector directing expression of the molecule has been introduced) are administered to the subject. Delivery methods are discussed further below.

It may advantageous to employ various nucleotide modifications and analogs to confer desirable properties on the antisense nucleic acid, ribozyme, siRNA, or shRNA. Numerous nucleotide analogs, nucleotide modifications, and modifications elsewhere in a nucleic acid chain are known in the art, and their effect on properties such as hybridization and nuclease resistance has been explored. For example, various modifications to the base, sugar and internucleoside linkage have been introduced into oligonucleotides at selected positions, and the resultant effect relative to the unmodified oligonucleotide compared. A number of modifications have been shown to alter one or more aspects of the oligonucleotide such as its ability to hybridize to a complementary nucleic acid, its stability, etc . For example, useful 2′-modifications include halo, alkoxy and allyloxy groups. U.S. Pat. Nos. 6,403,779; 6,399,754; 6,225,460; 6,127,533; 6,031,086; 6,005,087; 5,977,089, and references therein disclose a wide variety of nucleotide analogs and modifications that may be of use in the practice of the present invention. See also Crooke, S. (ed.), referenced above, and references therein. As will be appreciated by one of ordinary skill in the art, analogs and modifications may be tested using, e.g., the assays described herein or other appropriate assays, in order to select those that effectively reduce expression of the target nucleic acid. The analog or modification preferably results in a nucleic acid with increased absorbability (e.g., increased absorbability across a mucus layer, increased oral absorption, etc.), increased stability in the blood stream or within cells, increased ability to cross cell membranes, etc.

Antisense RNAs, ribozymes, siRNAs or shRNAs may be prepared by any method known in the art for the synthesis of nucleic acid molecules. These include techniques for chemical synthesis such as solid phase phosphoramidite chemical synthesis. In the case of siRNAs, the structure may be stabilized, for example by including nucleotide analogs at one or more free strand ends in order to reduce digestion, e.g., by exonucleases. This may also be accomplished by the use of deoxy residues at the ends, e.g., by employing dTdT overhangs at each 3′ end. Alternatively, antisense, ribozyme, siRNA or shRNA molecules may be generated by in vitro transcription of DNA sequences encoding the relevant molecule. Such DNA sequences may be incorporated into a wide variety of vectors with suitable RNA polymerase promoters such as T7, T3, or SP6.

Antisense, ribozyme, siRNA or shRNA molecules may be generated by intracellular synthesis of small RNA molecules, as described above, which may be followed by intracellular processing events. For example, intracellular transcription may be achieved by cloning templates into RNA polymerase III transcription units, e.g., under control of a U6 or H1 promoter. In one approach for intracellular synthesis of siRNA, sense and antisense strands are transcribed from individual promoters, which may be on the same construct. The promoters may be in opposite orientation so that they drive transcription from a single template, or they may direct synthesis from different templates. However, it may be prefererable to express a single RNA molecule that self-hybridizes to form a hairpin RNA that is then cleaved by DICER within the cell.

The antisense, ribozyme, siRNA, or shRNA molecules of the invention may be introduced into cells by any of a variety of methods. For instance, antisense, ribozyme, siRNA, or shRNA molecules or vectors encoding them can be introduced into cells via conventional transformation or transfection techniques. As used herein, the terms “transformation” and “transfection” are intended to refer to a variety of art-recognized techniques for introducing foreign nucleic acid (e.g., DNA or RNA) into a host cell, including calcium phosphate or calcium chloride co-precipitation, DEAE-dextran-mediated transfection, lipofection, injection, or electroporation.

Vectors that direct in vivo synthesis of antisense, ribozyme, siRNA, or shRNA molecules constitutively or inducibly can be introduced into cell lines, cells, or tissues. In certain preferred embodiments of the invention, inventive vectors are gene therapy vectors (e.g., adenoviral vectors, adeno-associated viral vectors, retroviral or lentiviral vectors, or various nonviral gene therapy vectors) appropriate for the delivery of a construct directing transcription of an siRNA to mammalian cells, most preferably human cells.

Preferred siRNA, shRNA, antisense, or ribozyme compositions reduce the level of a target transcript and its encoded protein by at least 2-fold, preferably at least 4-fold, more preferably at least 1 0-fold or more. The ability of a candidate siRNA to reduce expression of the target transcript and/or its encoded protein may readily be tested using methods well known in the art including, but not limited to, Northern blots, RT-PCR, microarray analysis in the case of the transcript, and various immunological methods such as Western blot, ELISA, immunofluorescence, etc., in the case of the encoded protein. In addition, the potential of any siRNA, shRNA, antisense, or ribozyme composition for treatment of a particular condition or disease associated with heart failure may also be tested in appropriate animal models or in human subjects, as is the case for all methods of treatment described herein. Appropriate animal models include mice, rats, rabbits, sheep, dogs, etc., with experimentally induced heart failure, e.g., due to coronary artery ligation, pacemaker-induced tachycardia, etc.

5. Delivery of Nucleic Acids to a Subject

The various nucleic acids described above (e.g., nucleic acids encoding UIR or DIR polypeptides, fragments, and variants; antisense oligonucleotides complementary to UIR or DIR mRNA, ribozymes designed to cleave UIR or DIR mRNA, siRNA or shRNA targeted to UIR or DIR mRNA may be delivered to a subject using any of a variety of approaches, including those applicable to non-nucleic acid agents such as IV, intranasal, oral, etc. However, according to certain embodiments of the invention the nucleic acids are delivered via a gene therapy approach, in which a construct capable of directing expression of one or more of the inventive nucleic acids is delivered to cells or to the subject (ultimately to enter cells, where transcription may occur). Thus according to certain embodiments of the invention the vectors described above include gene therapy vectors appropriate for the delivery of a construct that directs expression of a UIR or DIR polypeptide, variant, fragment, etc., or a construct directing transcription of an antisense oligonucleotide complementary to a UIR or DIR mRNA, or a ribozyme designed to cleave UIR or DIR mRNA, or an siRNA or shRNA targeted to a UIR or DIR mRNA to mammalian cells, more preferably cells of a domestricated mammal, and most preferably human cells. A variety of gene therapy vectors are known in the art. Suitable gene therapy vectors include viral vectors such as adenoviral or adeno-associated viral vectors, retroviral vectors and lentiviral vectors. In certain instances lentiviruses may be preferred due, e.g., to their ability to infect nondividing cells. See, e.g., Mautino and Morgan, AIDS Patient Care STDS 2002 January;16(1):11-26. See also Lois, C., et al., Science, 295: 868-872, Feb. 1, 2002, describing the FUGW lentiviral vector; Somia, N., et al. J. Virol. 74(9): 4420-4424, 2000; Miyoshi, H., et al., Science 283: 682-686, 1999; and U.S. Pat. No. 6,013,516.

A number of nonviral vectors and gene delivery systems exist, any of which may be used in the practice of the invention. For example, extrachromosomal DNA (e.g., plasmids) may be used as a gene therapy vector. See, e.g., Stoll, S. and Calor, M, “Extrachromosomal plasmid vectors for gene therapy”, Curr Opin Mol Ther, 4(4):299-305, 2002. According to one approach, the inclusion of appropriate genetic elements from various papovaviruses allows plasmids to be maintained as episomes within mammalian cells. Such plasmids are faithfully distributed to daughter cells. In particular, viral elements of various polyomaviruses and papillomaviruses such as BK virus (BKV), bovine papilloma virus 1 (BPV-1) and Epstein-Barr virus (EBV), among others, are useful in this regard. The invention therefore provides plasmids that direct expression of a UIR or DIR polypeptide, variant, fragment, etc., or a construct directing transcription of an antisense oligonucleotide complementary to a UIR or DIR mRNA, or a ribozyme designed to cleave UIR or DIR mRNA, or an siRNA targeted to a UIR or DIR mRNA to mammalian cells, preferably domesticated mammal cells, and most preferably human cells. According to certain embodiments of the invention the plasmids comprise a viral element sufficient for stable maintenance of the transfer plasmid as an episome within mammalian cells. Appropriate genetic elements and their use are described, for example, in Van Craenenbroeck, et al., Eur. J Biochem. 267, 5665-5678 (2000) and references therein, all of which are incorporated herein by reference. Plasmids can be delivered as “naked DNA” or in conjunction with a variety of delivery vehicles.

Protein/DNA polyplexes represent an approach useful for delivery of nucleic acids to cells and subjects. These vectors may be used to deliver constructs directing transcription of the inventive nucleic acids (constructs that direct transcription of UIR OR DIR polypeptides, fragments, or variants, antisense molecules, ribozymes, or siRNAs) or may be used to deliver the nucleic acids themselves. Thus their use is not limited to gene therapy. See, e.g., Cristiano, R., Surg. Oncol. Clin. N. Am., 11(3), 697-715, 2002. Cationic polymers and liposomes may also be used for these purposes. See, e.g., Merdan, T., et al., “Prospects for cationic polymers in gene and oligonucleotide therapy against cancer”, Adv Drug Deliv Res, 54(5), 715-58, 2002; Liu, F. and Huang, L., “Development of non-viral vectors for systemic gene delivery”, J. Control. Release, 78(1-3):259-66, 2002; Maurer, N., et al., “Developments in liposomal drug delivery systems”, Expert Opin Biol Ther, 1(2), 201-26, 2001; and Li, S. and Ma, Z., “Nonviral gene therapy”, Curr Gene Ther, 1(2), 201-26, 2001. See Rasmussen, H., Curr Opin Mol. Ther, 4(5), 476-81, 2002 for a review of angiogenic gene therapy strategies for the treatment of cardiovascular disease. Numerous reagents and methods for gene therapy are described in Philips, I., (ed.), Methods in Enzymology, Vol. 346: Gene Therapy Methods, Academic Press, 2002.

Any of the nucleic acid delivery vehicles (or nucleic acids themselves) can be targeted for delivery to specific cells, tissues, etc. In particular, they can be targeted to cardiac cells using antibodies or ligands that specifically bind to a UIR or DIR polypeptide as discussed further below. Nucleic acids can be directly conjugated to such antibodies or ligands, which then deliver the nucleic acids to cardiac cells.

Gene therapy protocols may involve administering an effective amount of a gene therapy vector comprising a nucleic acid capable of directing expression of a UIR or DIR polynucleotide, variant, or fragment, UIR or DIR antisense nucleic acid, or a ribozyme or siRNA targeted to a UIR or DIR mRNA to a subject. Another approach that may be used alternatively or in combination with the foregoing is to isolate a population of cells, e.g., stem cells or immune system cells from a subject, optionally expand the cells in tissue culture, and administer a gene therapy vector to the cells in vitro. The cells may then be returned to the subject. Optionally, cells expressing the desired polynucleotide, siRNA, etc., can be selected in vitro prior to introducing them into the subject. In some embodiments of the invention a population of cells, which may be cells from a cell line or from an individual who is not the subject, can be used. Methods of isolating stem cells, immune system cells, etc., from a subject and returning them to the subject are well known in the art. Such methods are used, e.g., for bone marrow transplant, peripheral blood stem cell transplant, etc., in patients undergoing chemotherapy.

In yet another approach, oral gene therapy may be used. For example, U.S. Pat. No. 6,248,720 describes methods and compositions whereby genes under the control of promoters are protectively contained in microparticles and delivered to cells in operative form, thereby achieving noninvasive gene delivery. Following oral administration of the microparticles, the genes are taken up into the epithelial cells, including absorptive intestinal epithelial cells, taken up into gut associated lymphoid tissue, and even transported to cells remote from the mucosal epithelium. As described therein, the microparticles can deliver the genes to sites remote from the mucosal epithelium, i.e. can cross the epithelial barrier and enter into general circulation, thereby transfecting cells at other locations.

B. Methods for Increasing Gene Expression

Additional methods for identifying compounds capable of modulating gene expression are described, for example, in U.S. Pat. No. 5,976,793. These methods may be either to identify compounds that increase gene expression or to identify compounds that decrease gene expression. The screening methods described therein are particularly appropriate for identifying compounds that do not naturally occur within cells and that modulate the expression of genes of interest whose expression is associated with a defined physiological or pathological effect within a multicellular organism. Additional methods for identifying agents that increase expression of genes are found in Ho, S., et al., Nature, 382, pp. 822-826, 1996, which describes homodimeric and heterodimeric synthetic ligands that allow ligand-dependent association and disassociation of a transcriptional activation domain with a target promoter to increase expression of an operatively linked gene.

Expression can also be increased by introducing additional copies of a coding sequence into a cell of interest, i.e., by introducing a nucleic acid comprising the coding sequence into the cell. Preferably the coding sequence is operably linked to regulatory signals such as promoters, enhancers, etc., that direct expression of the coding sequence in the cell. The nucleic acid may comprise a complete UIR or DIR gene, or a portion thereof, preferably containing the coding region of the gene. The nucleic acid may be introduced into cells grown in culture or cells in a subject using any suitable method, e.g., any of those described above.

C. Identifying Agents that Modulate Expression of a UIR or DIR Gene

Agents such as antisense molecules, siRNAs, shRNAs, ribozymes, other nucleic acids, peptides or polypeptides, small molecules, etc., can be tested to determine whether they modulate the expression of a UIR or DIR gene. The invention provides a method for identifying an agent that modulates expression of a UIR or DIR polynucleotide or polypeptide comprising steps of: (i) providing a sample comprising cells that express a UIR or DIR polynucleotide or polypeptide; (ii) contacting the cells with a candidate agent; (iii) determining whether the level of expression of the polynucleotide or polypeptide in the presence of the compound is increased or decreased relative to the level of expression or activity of the polynucleotide or polypeptide in the absence of the compound; and (iv) identifying the compound as a modulator of the UIR or DIR polynucleotide or polypeptide if the level of expression or activity of the UIR or DIR polynucleotide or polypeptide is higher or lower in the presence of the compound relative to its level of expression or activity in the absence of the compound.

Expression of a UIR or DIR polynucleotide or polypeptide can be measured using a variety of methods well known in the art in order to determine whether any candidate agent increases or decreases expression (or for other purposes). In general, any measurement technique capable of determining RNA or protein presence or abundance may be used for these purposes. For RNA such techniques include, but are not limited to, microarray analysis (For information relating to microarrays and also RNA amplification and labeling techniques, which may also be used in conjunction with other methods for RNA detection, see, e.g., Lipshutz, R., et al., Nat Genet., 21(1 Suppl):20-4, 1999; Kricka L., Ann. Clin. Biochem., 39(2), pp. 114 -129; Schweitzer, B. and Kingsmore, S., Curr Opin Biotechnol 2001 February;12(1):21-7; Vineet, G., et al., Nucleic Acids Research, 2003, Vol. 31, No. 4.; Cheung, V., et al., Nature Genetics Supplement, 21:15-19, 1999; Methods Enzymol, 303:179-205, 1999; Methods Enzymol, 306: 3-18, 1999; M. Schena (ed.), DNA Microarrays: A Practical Approach, Oxford University Press, Oxford, UK, 1999. See als U.S. Pat Nos. 5,242,974; 5,384,261; 5,405,783; 5,412,087; 5,424,186; 5,429,807; 5,436,327; 5,445,934; 5,472,672; 5,527,681; 5,529,756; 5,545,531; 5,554,501; 5,556,752; 5,561,071; 5,599,695; 5,624,711; 5,639,603; 5,658,734; 6,235,483; WO 93/17126; WO 95/11995; WO 95/35505; EP 742 287; EP 799 897; U.S. Pat. Nos. 5,514,545; 5,545,522; 5,716,785; 5,932,451; 6,132,997; 6,235,483; US Patent Application Publication 20020110827).

Other methods for detecting expression of UIR or DIR polynucleotides include Northern blots, RNAse protection assays, reverse transcription (RT)-PCR assays, real time RT-PCR (e.g., Taqman™ assay, Applied Biosystems), SAGE (Velculescu et al. Science, vol. 270, pp. 484-487, October 1995), Invader® technology (Third Wave Technologies), etc. See, e.g., Eis, P. S. et al., Nat. Biotechnol. 19:673 (2001); Berggren, W. T. et al., Anal. Chem. 74:1745 (2002), etc. Methods for detecting UIR or DIR polypeptides include, but are not limited to, immunoblots (Western blots), immunofluorescence, flow cytometry (e.g., using appropriate antibodies), mass spectrometry, and protein microarrays (Elia, G., Trends Biotechnol, 20(12 Suppl):S19-22, 2002, and reference therein).

D. Reagents and Methods for Modulating Functional Expression or Activity of a UIR or DIR Polypeptide

As discussed above, the invention provides methods for identifying ligands that modulate (e.g., increase or decrease) activity of a UIR or DIR polypeptide and methods for identifying agents that modulate expression of a UIR or DIR polynucleotide or polypeptide. More generally, the invention also provides a method for identifying an agent that modulates expression or activity of a UIR or DIR polynucleotide or polypeptide comprising steps of: (i) providing a sample comprising a UIR or DIR polynucleotide or polypeptide; (ii) contacting the sample with a candidate compound; (iii) determining whether the level of expression or activity of the polynucleotide or polypeptide in the presence of the compound is increased or decreased relative to the level of expression or activity of the polynucleotide or polypeptide in the absence of the compound; and (iv) identifying the compound as a modulator of the expression or activity of the UIR or DIR polynucleotide or polypeptide if the level of expression or activity of the UIR or DIR polynucleotide or polypeptide is higher or lower in the presence of the compound relative to its level of expression or activity in the absence of the compound. In certain embodiments of the method the sample comprises cells that express the UIR or DIR polypeptide. The agents to be screened include any of those discussed above. Agents identified according to the above methods may be further tested in subjects, e.g., humans or other animals. The subject may be normal or may be suffering from or at risk of heart failure of a condition or disease associated with heart failure. The test may involve determinining whether administration of the agent reduces or alleviates one or more symptoms or signs of heart failure or improves a prognostic variable such as exercise capacity.

IX. Diagnostic Applications

Genes identified as upregulated or downregulated in recovery from heart failure serve as diagnostic targets. The invention therefore provides a method for providing diagnostic or prognostic information related to heart failure or to a disease or condition associated with heart failure comprising steps of: (i) providing a subject in need of diagnostic or prognostic information related to heart failure or to a disease or condition associated with heart failure; and (ii) determining the level of expression or activity of a UIR or DIR polynucleotide or polypeptide in the subject or in a biological sample obtained from the subject. The method may further comprise the step of (iii) comparing the determined level of expression or activity with known level(s) determined previously in the subject or in normal subjects or in subjects with heart failure, or in a biological sample obtained from the subject or from normal subjects or from subjects with heart failure. The determined level of expression or activity can be correlated with values that have been associated with particular diagnostic categories (e.g., New York Heart Association classification of heart failure), disease outcomes, likelihood of responding positively to particular treatments, time to progression to a more severe state, etc. The information can be provided to the subject and/or used to guide therapeutic decisions, e.g., the advisability of initiating or terminating various therapies, etc. By “normal subject” is meant a subject not suffering from heart failure or from a disease or clinical condition associated with heart failure as determined using a classification method accepted in the art, e.g., the New York Heart Association classification scheme, which divides subjects into normal or class 1, 2, 3, or 4, with increasing number indicating increasing severity of disease. The classification method may be based on clinical criteria, laboratory criteria, qualitative and/or quantitative tests including imaging tests, etc. For example, ejection fraction can be used to classify subjects, wherein normal is defined as a left ventricular ejection fraction greater than 45%, mild to moderate is 25% to 45%, and severe is less than 25%.

According to certain embodiments of the invention, a level of expression or activity of a DIR polynucleotide or polypeptide that is higher than would be expected in a normal subject or in a biological sample obtained from a normal subject, indicates an increased likelihood that the subject is at risk of or suffering from heart failure or a disease or condition associated with heart failure. A level of expression or activity of a DIR polynucleotide or polypeptide that is higher in the subject or in a biological sample obtained from the subject than the level determined previously for that subject indicates that the subject's disease has become more severe and/or that the subject has not responded to therapy. According to certain embodiments of the invention the level of expression of a DIR polynucleotide or polypeptide is an indicator of the severity of heart failure or of a disease or condition associated with heart failure, with a higher level, e.g., relative to normal being indicative of greater severity.

According to certain embodiments of the invention, a level of expression or activity of a DIR polynucleotide or polypeptide that is lower than would be expected in a subject with heart failure or in a biological sample obtained from a subject with heart failure, indicates a decreased likelihood that the subject is at risk of or suffering from heart failure or a disease or condition associated with heart failure. A level of expression or activity of a DIR polynucleotide or polypeptide that is lower in the subject or in a biological sample obtained from the subject than the level determined previously for that subject indicates that the subject's disease has become less severe and/or that the subject has responded to therapy. According to certain embodiments of the invention the level of expression of a DIR polynucleotide or polypeptide is an indicator of the severity of heart failure or of a disease or condition associated with heart failure, with a lower level, e.g., relative to that typically found in heart failure, being indicative of lower severity.

According to certain embodiments of the invention, a level of expression or activity of a UIR polynucleotide or polypeptide that is lower than would be expected in a normal subject or in a biological sample obtained from a normal subject, indicates an increased likelihood that the subject is at risk of or suffering from heart failure or a disease or condition associated with heart failure. A level of expression or activity of a UIR polynucleotide or polypeptide that is lower in the subject or in a biological sample obtained from the subject than the level determined previously for that subject indicates that the subject's disease has become more severe and/or that the subject has not responded to therapy. According to certain embodiments of the invention the level of expression of a UIR polynucleotide or polypeptide is an indicator of the severity of heart failure or of a disease or condition associated with heart failure, with a lower level, e.g., relative to normal being indicative of greater severity.

According to certain embodiments of the invention, a level of expression or activity of a UIR polynucleotide or polypeptide that is higher than would be expected in a subject with heart failure or in a biological sample obtained from a subject with heart failure, indicates a decreased likelihood that the subject is at risk of or suffering from heart failure or a disease or condition associated with heart failure. A level of expression or activity of a UIR polynucleotide or polypeptide that is higher in the subject or in a biological sample obtained from the subject than the level determined previously for that subject indicates that the subject's disease has become less severe and/or that the subject has responded to therapy. According to certain embodiments of the invention the level of expression of a UIR polynucleotide or polypeptide is an indicator of the severity of heart failure or of a disease or condition associated with heart failure, with a higher level, e.g., relative to that found in subjects with heart failure, being indicative of lesser severity.

In any of the foregoing methods the level of expression of an expression product (e.g., an RNA transcribed from a gene or a polypeptide encoded by such an RNA) can be determined according to standard methods, some of which are described elsewhere herein. For example, a sample of cardiac tissue (cardiac biopsy) can be obtained. Such biopsies are routinely performed, e.g., to assess rejection following cardiac transplant. Endocardial or myocardial biopsies can be done using a catheter inserted into the heart via the jugular vein. RNA can be detected using in situ hybridization or extracted and measured, optionally being amplified prior to measurement. RT-PCR can be used. Protein expression can be measured using various immunological techniques including immunohistochemistry, immunoblot, immunoassays such as ELISA assays, etc.

Rather than determining the level of expression of a polynucleotide or polypeptide, in certain embodiments of the invention the functional activity of the polypeptide is measured. For example, in the case of a kinase such as MAPK4 or TEC, kinase activity can be measured. Methods for doing so are well known in the art and can utilize either endogenous substrates or synthetic substrates, e.g., substrates containing consensus sequences for phosphorylation for either serine/threonine or tyrosine kinases. Activity of other polypeptides having known biological and/or enzymatic activities can be measured using any of a variety of methods known in the art, as appropriate for the particular activity.

Instead of determining the expression level or activity of a polynucleotide or polypeptide in a sample obtained from a subject, the expression level can be measured using imaging as described above. Activity can also be measured using imaging techniques, e.g., by targeting a substrate for an enzymatic reaction catalyzed by the polypeptide to cardiac cells and monitoring conversion of the substrate into product by performing sequential imaging. Labeled substrates can be used to facilitate such monitoring. Methods for performing functional imaging, either invasively or noninvasively, are known in the art.

In the case of certain diagnostic targets, the polypeptide encoded by the gene is secreted from cells and circulates in the bloodstream. In such cases the level of expression or activity of the gene product can be measured in a blood or serum sample obtained from the subject. Polypeptides that are secreted by cells typically include a signal sequence that directs their secretion. In addition, certain of the gene products encode receptors. The invention also provides diagnostic methods based on the measurement of levels of endogenous ligands for these receptors. According to certain embodiments of the invention the level of an endogenous ligand for a UIR or DIR polypeptide is measured instead of or in addition to the level of expression or activity of the corresponding UIR or DIR polypeptide. For example, as further described below, measurement of circulating apelin levels correlates with disease severity in heart failure. The level of the ligand can be measured using any suitable method, e.g., radioimmunoassay, ELISA, functional assays, etc.

Thus the invention provides a method for providing diagnostic or prognostic information related to heart failure or to a disease or condition associated with heart failure comprising steps of: (i) providing a subject in need of diagnostic or prognostic information related to heart failure or to a disease or condition associated with heart failure; and (ii) determining the level of a ligand for a UIR or DIR polypeptide in the subject or in a biological sample obtained from the subject. The method may further comprise the step of (iii) comparing the determined level with known level(s) determined previously in the subject or in normal subjects or in subjects with heart failure, or in a biological sample obtained from the subject or from normal subjects or from subjects with heart failure. The determined level of the ligand can be correlated with values that have been associated with particular diagnostic categories (e.g., New York Heart Association (NYHA) classification of heart failure), disease outcomes, likelihood of responding positively to particular treatments, time to progression to a more severe state, etc. The information can be provided to the subject and/or used to guide therapeutic decisions, e.g., the advisability of initiating or terminating various therapies, etc.

According to certain embodiments of the invention, a level of expression or activity of a ligand for a DIR polypeptide that is higher than would be expected in a normal subject or in a biological sample obtained from a normal subject, indicates an increased likelihood that the subject is at risk of or suffering from heart failure or a disease or condition associated with heart failure. A level of ligand for a DIR polynucleotide or polypeptide that is higher in the subject or in a biological sample obtained from the subject than the level determined previously for that subject indicates that the subject's disease has become more severe and/or that the subject has not responded to therapy. According to certain embodiments of the invention the level of a ligand for a DIR polypeptide is an indicator of the severity of heart failure or of a disease or condition associated with heart failure, with a higher level, e.g., relative to normal being indicative of greater severity.

According to certain embodiments of the invention, a level of a ligand for a DIR polypeptide that is lower than would be expected in a subject with heart failure or in a biological sample obtained from a subject with heart failure, indicates a decreased likelihood that the subject is at risk of or suffering from heart failure or a disease or condition associated with heart failure. A level of a ligand for a DIR polypeptide that is lower in the subject or in a biological sample obtained from the subject than the level determined previously for that subject indicates that the subject's disease has become less severe and/or that the subject has responded to therapy. According to certain embodiments of the invention the level of a ligand for a DIR polypeptide is an indicator of the severity of heart failure or of a disease or condition associated with heart failure, with a lower level, e.g., relative to that typically found in heart failure, being indicative of lower severity.

According to certain embodiments of the invention, a level of a ligand for a UIR polypeptide that is lower than would be expected in a normal subject or in a biological sample obtained from a normal subject, indicates an increased likelihood that the subject is at risk of or suffering from heart failure or a disease or condition associated with heart failure. A level of a ligand for a UIR polypeptide that is lower in the subject or in a biological sample obtained from the subject than the level determined previously for that subject indicates that the subject's disease has become more severe and/or that the subject has not responded to therapy. According to certain embodiments of the invention the level of a ligand for a UIR polypeptide is an indicator of the severity of heart failure or of a disease or condition associated with heart failure, with a lower level, e.g., relative to normal being indicative of greater severity.

According to certain embodiments of the invention, a level of a ligand for a UIR polypeptide that is higher than would be expected in a subject with heart failure or in a biological sample obtained from a subject with heart failure, indicates a decreased likelihood that the subject is at risk of or suffering from heart failure or a disease or condition associated with heart failure. A level of a ligand for a UIR polypeptide that is higher in the subject or in a biological sample obtained from the subject than the level determined previously for that subject indicates that the subject's disease has become less severe and/or that the subject has responded to therapy. According to certain embodiments of the invention the level of a ligand for a UIR polypeptide is an indicator of the severity of heart failure or of a disease or condition associated with heart failure, with a higher level, e.g., relative to that found in subjects with heart failure, being indicative of lesser severity.

As a particular example, the invention provides a method of providing diagnostic or prognostic information related to heart failure or to a disease or condition associated with heart failure comprising steps of: (i) providing a subject in need of diagnostic or prognostic information related to heart failure or to a disease or condition associated with heart failure; and (ii) determining the level of apelin in the subject or in a biological sample obtained from the subject. The method may further comprise the step of (iii) comparing the determined apelin level with known level(s) determined previously in the subject or in normal subjects or in subjects with heart failure, or in a biological sample obtained from the subject or from normal subjects or from subjects with heart failure. The sample, can be, e.g., a blood, plasma, or serum sample. Any apelin peptide can be measured, e.g., apelin-12, apelin-13, or PYR-apelin-13. The measurement can be performed, using for example, a radioimmunoassay or ELISA, etc.

As described in Example 5, the plasma level of apelin is correlated with particular diagnostic categories for heart failure. In particular, there are significant increases in the plasma level of apelin in early heart failure through NYHA class 2, while in later stages (class 3-4), the mean level is lower. Thus apelin levels rise in mild to moderate disease but fall in severe disease. The level of apelin may thus be used to distinguish patients suffering from mild to moderate disease with normal subjects and those suffering from severe disease. It may be particularly useful to monitor apelin levels over time. For example, if the apelin level in a subject initially classified as normal begins to rise, this may be indicative of progression to mild or moderate heart failure. If the apelin level in a subject initially classified as having severe heart failure begins to rise, this may be indicative that the subject's condition is improving to a mild or moderate disease severity. In general, it may be desirable to consider apelin level together with other indicators of disease severity. For example, if an initial measurement of apelin level indicates that the individual has an apelin level that is consistent with either normal or severe disease, clinical and/or other criteria will generally allow the subject to be unambiguously assigned to either the normal or severe category. The apelin level may be used thereafter to more accurately quantify the subject's disease state and/or monitor the response to treatment. The apelin level can be provided to the subject and/or used to guide therapeutic decisions, e.g., the advisability of initiating or terminating various therapies, etc. It is noted that plasma levels of another endogeonous peptide, brain natriuretic peptide (BNP) are used clinically as a diagnostic tool in human heart failure (Hobbs, R. E., “Using BNP to diagnose, manage, and treat heart failure”, Cleveland Clinic Journal of Medicine, 70(4): 333-336 (2003); Bhatia, V., Nayyar, P., and Dhinda, S., “Brain natriuretic peptide in diagnosis and treatment of heart failure”, J. Postgrad Med, 49(2): 182-5 (2003)).

X. Therapeutic Applications and Screening Methods

A. UIR and DIR Genes and Polypeptides as Therapeutic Targets

As discussed above, the discovery that expression of UIR and DIR genes is upregulated or downregulated, respectively, following mechanical offloading in heart failure suggests that these genes and their expression products are appropriate targets for treatment or prevention of heart failure and diseases and clinical conditions associated with heart failure (including, but are not limited to atherosclerosis, hypertension, restenosis, ischemic cardiovascular diseases, idiopathic or viral cardiomyopathy, diabetes, peripheral arterial disease, etc.). Thus the invention provides a method for treating heart failure or a disease or clinical condition associated with heart failure comprising: (i) providing a subject at risk of or suffering from a disease or clinical condition associated with heart failure; and (ii) administering a compound that modulates expression or activity of a UIR or DIR polynucleotide or polypeptide to the subject. The invention further provides a method for treating heart failure or a disease or clinical condition associated with heart failure comprising: (i) providing a subject at risk of or suffering from a disease or clinical condition associated with heart failure; and (ii) administering a compound that modulates an endogenous ligand for a UIR or DIR polypeptide to the subject. By “modulate” is meant to enhance or reduce the level or activity of a molecule or to alter the temporal or spatial pattern of its expression or activity, in various embodiments of the invention. For example an agent that acts as an agonist or antagonist at a particular receptor is considered to modulate the receptor.

A variety of methods of modulating the expression or activity of UIR or DIR gene expression products and/or ligands are provided above. Any of the agents identified according to such methods may be used to modulate expression or activity of the UIR or DIR gene expression products and/or ligands for therapeutic or other purposes.

In particular, the invention provides a method of treating heart failure or a disease or condition associated with heart failure comprising the step of administering a compound that increases functional activity of the APJ receptor. One such compound the apelin-12 peptide. Other suitable compounds include peptides whose sequence comprises the sequence of apelin-12. As mentioned above, a variety of peptides that are cleaved from the apelin precursor in vivo are known and can be used. Such compounds may include modifications, either modifications that take place in vivo or modifications that are introduced by the hand of man. Various modifications that can be made in polypeptides are described above. One preferred compound is Pyr-apelin-13, which is pyroglutamylated apelin and is the predominant form circulating in the body and may be more stable.

Apelin may be administered in any of a variety of ways including subcutaneously, intramuscularly, intravenously, intraperitoneally, inhalationally, etc. Apelin may be administered as a bolus or as a continuous infusion over a period of time. An implantable pump may be used. In certain embodiments of the invention, intermittent or continuous apelin administration is continued for one to several days (e.g., 2-3 or more days), or for longer periods of time, e.g., weeks, months, or years. It may be desirable to maintain an average plasma apelin concentration above a particular threshold value either during administration or between administration of multiple doses. A desirable concentration may be determined, for example, based on the subject's physiological condition, disease severity, etc. Such desirable value(s) can be identified by performing standard clinical trials. In certain embodiments of the invention a desirable plasma apelin level is greater than the average normal level (e.g., the average level in normal subjects matched for variables such as age, sex, weight, etc., but not suffering from heart failure) by a factor of at least 2, at least 5, at least 10, at least 20, at least 30, at least 40, at least 50, or more.

As described in Example 7, the inventors have studied the effects of both acute and chronic apelin administration on various hemodynamic parameters in normal intact animals and have found a number of striking results. For example, acute administration of apelin in vivo caused a reduction in left ventricular end diastolic area and an increase in left ventricular elastance, whereas chronic apelin infusion increased load independent contractility without increasing left ventricular (LV) mass. These findings suggest an important role for the apelin-APJ system in cardiovascular control. Without wishing to be bound by any theory, the inventors hypothesized that apelin would reduce both left ventricular preload and afterload through venous and arterial dilation. The finding that end diastolic area is significantly decreased after intraperitoneal injection of apelin-12 provides the first demonstration that the vascular reactivity of apelin couples to the left ventricle. Decreases in maximum and end systolic pressure were detected in invasive studies, in line with previously observed changes in mean arterial pressure. While not wishing to be bound by any theory, the inventors propose that the significant increase in heart rate seen in the MRI study described in Example 7 is likely explained by a baroreceptor mediated response to decreased mean arterial pressure.

In the isolated rat heart, Szokodi et al (referenced above) described a positive, efficacious and potent effect of apelin on externally developed tension and pre-load recruitable maximum rate of developed pressure, however these effects had not been observed in vivo. The findings presented herein, i.e., that apelin increases the slope of the end systolic pressure-volume relationship (ventricular elastance), the slope of the end diastolic volume to stroke work relationship and, in the chronic infusion model, the velocity of circumferential shortening, provide the first demonstration of an in vivo effect of apelin on myocardial contractility.

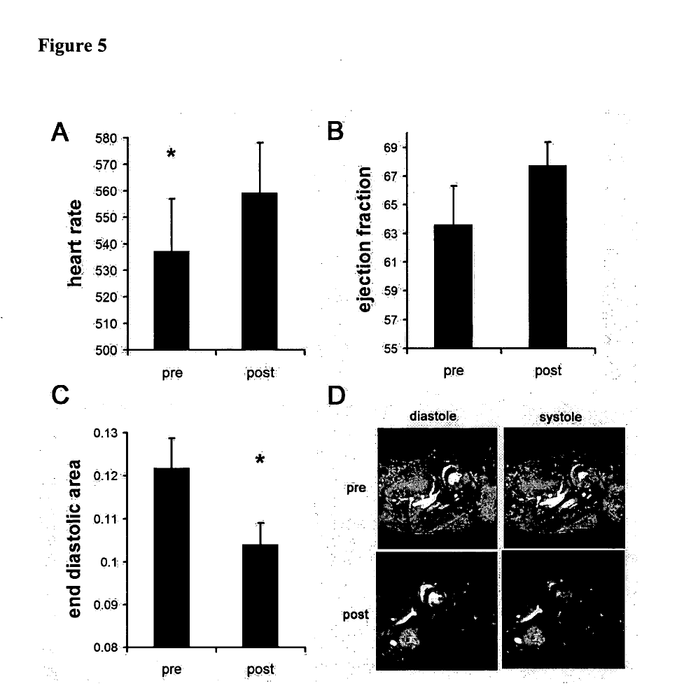

The change in slope of the end-systolic pressure-volume relationship (FIG. 5, Panel C) is consistent with the change in the end diastolic pressure to dP/dtₘₐₓ relationship observed by Szokodi et al. Further, this observation reinforces the importance of assessing biological signaling systems active at the level of the myocardium and vasculature in an integrated manner: standard, load dependent measures of contractility such as dP/dtₘₐₓ and ejection fraction were not significantly different after apelin infusion in vivo. While not wishing to be bound by any theory, the inventors propose that these observations can be clearly understood in the context of previously described changes in loading conditions. While apelin increases intrinsic contractility, an effect which, in the absence of changes in loading conditions, would lead to an increase in ejection fraction and dP/dtₘₐₓ, apelin mediated reductions in preload will move the Frank Starling curve to the left and thus, alter ejection phase indices (and particularly those such as dP/dtₘₐₓ which are exquisitely sensitive to preload) downwards. Similarly, although apelin mediated reductions in systemic blood pressure reduce afterload, something which in itself would tend to augrnent afterload dependent measures of contractility such as ejection fraction, the leftward shift in the Frank Starling relationship will mitigate the increase and result in a net effect equivalent to little change or little increase. This is, in fact, what is observed. Effects of agents which change loading conditions concurrent with the visco-elastic properties of the ventricle (and there is significant conservation in signaling pathways between the vasculature and the myocardium) can be masked by traditional measures of contractility.

Effecting a reduction in cardiac loading while increasing contractile reserve makes the apelin-APJ system an attractive target for therapy in heart failure. As described herein, increases in the myocardial expression of both apelin and its receptor APJ were observed following LVAD offloading in human heart failure. Further, increases in circulating apelin occur in patients with moderate LV dysfunction. Together, these observations suggest that apelin may act as a ‘good peptide’ in heart failure (akin to the natriuretic peptides) serving to ameliorate rather than antagonize the abnormal hemodynamic state of that disease.

A caveat to chronic pharmacologic augmentation of a positive inotropic is the potential for deleterious effects demonstrated in clinical trials with agents such as milrinone (Curfman G D. Inotropic therapy for heart failure--an unfulfilled promise. N Engl J Med., 325:1509-10, 1991; Packer M, Carver J R, Rodeheffer R J, Ivanhoe R J, DiBianco R, Zeldis S M, Hendrix G H, Bommer W J, Elkayam U, Kukin M L, et al. Effect of oral milrinone on mortality in severe chronic heart failure. The PROMISE Study Research Group. N Engl J Med., 325:1468-75, 1991) and dobutamine (Elis A, Bental T, Kimchi O, Ravid M, Lishner M. Intermittent dobutamine treatment in patients with chronic refractory congestive heart failure: a randomized, double-blind, placebo-controlled study. Clin Pharmacol Ther., 63:682-5, 1998; Felker G M, O'Connor C M. Inotropic therapy for heart failure: an evidence-based approach. Am Heart J., 142:393-401, 2001) and in transgenic models overexpressing components of the beta-adrenergic signaling systems (Du X J, Gao X M, Wang B, Jennings G L, Woodcock E A, Dart A M. Age-dependent cardiomyopathy and heart failure phenotype in mice overexpressing beta(2)-adrenergic receptors in the heart. Cardiovasc Res., 48:448-54, 2000; Engelhardt S, Hein L, Dyachenkow V, Kranias E G, Isenberg G, Lohse M J. Altered calcium handling is critically involved in the cardiotoxic effects of chronic beta-adrenergic stimulation, Circulation, 109:1154-60, 2004).

However, apelin increases contractile reserve through an increase in elastance and concomitantly decreases loading, so does not overdrive the heart. It has been known for over a decade that the hemodynamic profile of well characterized inotropic agents is improved by the addition of pre-load reducing agents (Verma S P, Silke B, Reynolds G W, Richmond A, Taylor S H. Modulation of inotropic therapy by venodilation in acute heart failure: a randomised comparison of four inotropic agents, alone and combined with isosorbide dinitrate. J Cardiovasc Pharmacol. 19:24-33, 1992).

The inventors further demonstrated that no increase in heart or ventricular weight occurs after two weeks of apelin infusion compared to saline control. This is despite increases in cardiac output and in the velocity of circumferential shortening (Szokodi, et al.), despite significant homology between apelin and angiotensin II, and despite similarity in downstream signaling networks to endothelin, angiotensin II and alpha-adrenoceptor agonists, all of which might suggest apelin would be involved in remodeling and might cause cardiac hypertrophy via calcium dependent processes such as calmodulin kinase activation (Bers D M. Excitation contraction coupling. 2nd edition ed: Kluwer Academic Publishers; 2001). However, whereas endothelin and angiotensin increase peripheral resistance, apelin is one of the most potent arterial and venous dilators known, and it seems likely that this more global effect of apelin on vascular tone outweighs any local tissue effects leading to a net absence of hypertrophy.

In addition, the results presented herein describe protein expression of APJ by myocardial cells of the atrium and ventricle for the first time and also identify apelin expression in the coronary endothelium. Without wishing to be bound by any theory, the inventors propose that these data establish a paracrine signaling pathway that links the endothelial cells and myocardial cells for the purpose of regulating cardiac contractility. In addition, the data showing protein and mRNA level expression of apelin and APJ by myocardial cells in the embryonic heart suggest an autocrine pathway that is important for heart development, and which is functional before establishment of the coronary circulation. Since apelin expression by adult myocardial cells was not observed, there appears to be a shift of apelin expression from myocardial to endothelial cells after establishment of the coronary circulation in late gestation. Quantitative evaluation of mRNA levels through late gestation and adulthood indicated that apelin expression is relatively constant, and is consistent with a need to maintain apelin levels for cardiac homeostasis. In decompensated failing human heart tissues apelin immunoreactivity was noted in association with myocardial cells, suggesting that this embryonic pathway is reactivated in the setting of congestive heart failure, in parallel with other embryonic programs.

In summary, these experiments are highly significant for a number of reasons. Firstly, previous work using apelin was performed either on isolated cardiac preparations or using acute administration such as a single intravenous dose. Results obtained using isolated tissue preparations cannot be readily interpreted or extrapolated to effects that would result in vivo since the isolated tissue is no longer subject to the influence of endogenous regulatory systems and feedback found in the body. Thus it is virtually impossible to extrapolate from results obtained using isolated tissues to conditions existing in an intact subject. The experiments described herein establish that administration of apelin results in favorable changes in hemodynamic parameters in normal subjects, strongly suggesting that similar favorable changes will result in subjects with heart failure or an associated disease or condition. For example, chronic apelin administration resulted in reduced left ventricular preload and afterload, increased contractile reserve, and a significant increase in cardiac output. In addition, the experiments show that chronic apelin administration did not result in hypertrophy, a significant consideration in selecting an appropriate therapy for subjects with heart failure, since cardiac hypertrophy is generally undesirable.

In addition to the experiments described above and in Example 7, the inventors administered apelin by chronic infusion to mice with experimentally induced heart failure (Example 8). Results showed a marked, statistically significant increase in exercise capacity, the most significant prognostic indicator in human heart failure and a variable that is widely used to assess the severity of cardiovascular disease.

According to certain embodiments of the invention apelin is administered chronically in an amount effective to cause an improvement in at least one clinical symptom, laboratory sign, or diagnostic criterion, of heart failure. By “chronic administration” is meant that the level of apelin (either peak or average plasma level) is maintained above a preselected value for at least 24 hours. In various embodiments of the invention chronic administration refers to a period of at least 36 hours, at least 2 days (48 hours), at least 3 days, at least 4 days, at least 1 week, at least 2 weeks, at least 3 weeks, at least 4 weeks, or longer, e.g., 1 to several months (2, 3, 4, 6 months), years, etc. In various embodiments of the invention the period of chronic administration may be interrupted by one or more periods during which apelin is not administered or is administered at a dose insufficient to reach the predetermined desirable level. However, generally such level would be reached at least 25%, at least 50%, at least 75%, at least 90%, or more of the time over which apelin is administered. In preferred embodiments of the invention apelin is administered chronically in an amount sufficient to cause an improvement in at least one hemodynamic parameter. The improvement can be, for example, a reduction in ventricular preload (e.g., a reduction in left and/or right ventricular preload), a reduction in ventricular afterload (e.g., a reduction in left and/or right ventricular afterload), a decrease in pulmonary artery pressure, an increase in contractile reserve, an increase in cardiac output, an increase in exercise capacity, etc.

Apelin can be administered alone or in combination with any of a variety of other agents used in the treatment of heart failure. A number of such agents are mentioned above and these and other such agents are described in more detail in the scientific literature and in standard texts, for example, in Goodman & Gilman (referenced above) and in Braunwald, et al. (referenced above). By “in combination” is meant that the compounds are administered within a window of time such that they achieve effective concentrations in the body at the same time. The compounds need not be administered at the same time or as components of a single therapeutic composition. In certain embodiments of the invention apelin is administered in combination with BNP. It is noted that an intravenous formualation of BNP (nesiritide) has been approved for treatment of decompensated heart failure in hospital and emergency room settings, and its use in other contexts is being explored (Bhatia, et al; Hobbs, et al.)

XI. Pharmaceutical Compositions

The invention provides a variety of pharmaceutical compositions. For example, the invention provides pharmaceutical compositions containing antisense nucleic acids, siRNA, shRNA, ribozymes, or vectors for endogenous expression of such nucleic acids. The invention further provides a pharmaceutical composition comprising an effective amount of an antibody that specifically binds to a UIR or DIR polypeptide and a pharmaceutically acceptable carrier. The invention further provides a pharmaceutical composition comprising an effective amount of a ligand that specifically binds to a UIR or DIR polypeptide, and a pharmaceutically acceptable carrier. The antibodies and ligands may be conjugated with any of the therapeutic moieties discussed above.

In particular, the invention provides pharmaceutical compositions comprising apelin, e.g., apelin-12, apelin-13, Pyr-apelin-13, and other apelin peptides for the treatment and/or prevention of heart failure or a condition or disease associated with heart failure.

Compositions containing antibodies, ligands, conjugates, antisense nucleic acids, siRNA, shRNA, ribozymes, vectors for endogenous expression of nucleic acids such as siRNAs, shRNAs, ribozymes, antisense molecules, peptides, and/or small molecules or other therapeutic agents as described herein may be formulated for delivery by any available route including, but not limited to parenteral (e.g., intravenous), intradermal, subcutaneous, oral (e.g., inhalation), transdermal (topical), transmucosal, rectal, and vaginal. Preferred routes of delivery include parenteral, transmucosal, rectal, and vaginal. Inventive pharmaceutical compositions typically include one or more therapeutic agents, in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions. Compositions can also be delivered directly to a site of tissue injury or surgery. They may be administered by catheter or using diagnostic/therapeutic equipment such as bronchoscopes, colonoscopes, etc. Inventive compositions may also be delivered as implants or components of implantable devices. For example, inventive compositions may be used to coat stents and/or vascular grafts.

A pharmaceutical composition is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. Preferred pharmaceutical formulations are stable under the conditions of manufacture and storage and must be preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the inventive therapeutic agents are preferably delivered in the form of an aerosol spray from pressured container or dispenser which contains a suitable propellant, e.g., a gas such as carbon dioxide, or a nebulizer. It is noted that the lungs provide a large surface area for systemic delivery of therapeutic agents. The agents may be encapsulated, e.g., in polymeric microparticles such as those described in U.S. publication 20040096403, or in association with any of a wide variety of other drug delivery vehicles that are known in the art. In other embodiments of the invention the agents are delivered in association with a charged lipid as described, for example, in U.S. publication 20040062718. It is noted that the latter system has been used for administration of a therapeutic polypeptide, insulin, demonstrating the utility of this system for administration of peptide agents.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fisidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography, mass spectrometry, etc.

A therapeutically effective amount of a pharmaceutical composition typically ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The pharmaceutical composition can be administered at various intervals and over different periods of time as required, e.g., one time per week for between about 1 to 10 weeks, between 2 to 8 weeks, between about 3 to 7 weeks, about 4, 5, or 6 weeks, etc. For certain conditions it may be necessary to administer the therapeutic composition on an indefinite basis to keep the disease under control. The skilled artisan will appreciate that certain factors can influence the dosage and timing required to effectively treat a subject, including but not limited to the severity of the disease or disorder, previous treatments, the general health and/or age of the subject, and other diseases present. Generally, treatment of a subject with a therapeutic agent as described herein, can include a single treatment or, in many cases, can include a series of treatments.

Exemplary doses include milligram or microgram amounts of the inventive therapeutic agent per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.) It is furthermore understood that appropriate doses of a therapeutic agent depend upon the potency of the agent, and may optionally be tailored to the particular recipient, for example, through administration of increasing doses until a preselected desired response is achieved. It is understood that the specific dose level for any particular animal subject may depend upon a variety of factors including the activity of the specific compound employed, the age, body weight, general health, gender, and diet of the subject, the time of administration, the route of administration, the rate of excretion, any drug combination, and the degree of expression or activity to be modulated.

Inventive pharmaceutical compositions can be included in a container, pack, or dispenser together with instructions for administration.

### Exemplification

### EXAMPLE 1

### Identification of Genes Differentially Expressed Following LVAD Implantation

Materials and Methods

Implantation of left ventricular assist device. The Novacor Left Ventricular Assist System (World Heart Corporation, Ottawa, Ontario, Canada) was implanted providing a left ventricular apical core (pre-LVAD). The post-implant tissue sample was dissected from the left ventricle following recipient cardiectomy. Normal left ventricular tissue was derived from a patient with no history of coronary disease or cardiomyopathy.

RNA isolation and hybridization. RNA isolation and hybridization were performed as previously described (Ho, M. et al. Identification of endothelial cell genes by combined database mining and microarray analysis. Physiol Genomics (2003)). Common reference RNA was Universal Pooled Human Reference RNA, (Stratagene, La Jolla, Calif.). Samples were hybridized to the Agilent Human 1 Catalog Array. Arrays were washed and spun dry. A total of 44 hybridizations were performed on 11 pairs of pre- and post-LVAD RNA samples. Replicate hybridizations were performed as dye swaps.

Scanning, background subtraction and normalization of microarray data. Microarrays were scanned on an Agilent G2565AA Microarray Scanner System. Images were quantified using Agilent Feature Extraction Software (Version A.6.1.1). Processing included local background subtraction and a rank consistency based probe selection filter. Normalization was carried out using a LOWESS algorithm (Tseng, G. C., Oh, M. K., Rohlin, L., Liao, J. C. & Wong, W. H. Issues in cDNA microarray analysis: quality filtering, channel normalization, models of variations and assessment of gene effects. Nucleic Acids Res 29, 2549-57 (2001)). Dye-normalized signals of cy3 and cy5 channels were used in calculating log ratios. Ratios were averaged for each dye swap using the arithmetic mean.

Significance analysis of microarrays. This was performed as described previously (Tusher, V. G., Tibshirani, R. & Chu, G. Significance analysis of microarrays applied to the ionizing radiation response. Proc Natl Acad Sci U S A 98, 5116-21 (2001)). Heatmaps were generated using software available from the Quertermous lab web site at http://mozart.stanford.edu/TQLab/lvad/index.html. To generate heatmaps, output from the Significance Analysis of Microarrays was processed and then plotted using a software program written in Perl. The program Samster, available at http://falkow.stanford.edu/whatwedo/software/software.html (Charlie Kim, Falkow laboratory, Stanford University) generates a text file of raw data associated with the significantly differentially regulated genes determined by the Excel/Java version of SAM (available from Rob Tibshirani at the web site having URL www-stat.stanford.edu/%7Etibs/SAM/index.html).

A script entitled HeatMAP is then used to create the jpg or png file. This script is freely available from the Quertermous lab web site (see web site having URL quertermous.stanford.edu/people/mary.htm). It uses the GD.perl module from Lincoln Stein (see web site having URL stein.cshl.org/WWW/software/GD/index.html) and implements a row normalization algorithm for red-green color allocation.

Rank consistency score. The Rank consistency analysis and the corresponding output tables were produced by “BioTools”—software package for genomic data analysis developed at Agilent Labs, Palo Alto. For each patient k, the change of expression between averaged post and averaged pre LVAD samples for every gene g is calculated. These differences are ranked within each patient, in descending order. The rank of the gene g in patient k is denoted R_{g,k}. For every gene g, the rank consistency score S_{g,n} for all n patients is the maximal (i.e. the worst) rank of this gene among all patients,
S_{g,m}=max1_{≦k≦n}R_{g,k}/N,
where N is the total number of genes. Similarly, we can also compute the rank consistency score S_{g,m} for m out of n patients. In this case, for each patient we rank genes as before. For each gene we order its ranks, and then the score S_{g,m} corresponds to the m-th best rank:
S_{g,m}=m-th smallest R_{g,k}/N, 1≦k≦n
To determine the statistical significance of this score, we compute the P value of gene g with score s for m out of n patients. This is done under the null model of uniform and independent rank vectors. Therefore:

$$P value ( g , s ) = {\sum }_{k = m}^{n} ( \begin{matrix}n \\ k\end{matrix} ) {{s}^{k} ( 1 - s )}^{n - k} , where s = {S}_{g , m .}$$

Using these computed P values, we can estimate false discovery and binomial surprise rates (Reiner, A., Yekutieli, D. & Benjamini, Y. Identifying differentially expressed genes using false discovery rate controlling procedures. Bioinformatics 19, 368-75, 2003); Ben-Dor, A. et al. Tissue classification with gene expression profiles. J Comput Biol 7, 559-83, 2000). These compare the observed number of genes with score s or better, and the expected number of genes with such scores.

Hierarchical clustering. Unsupervised, average-linkage, hierarchical clustering was carried out using Cluster software (Eisen, et al., referenced above) entering all patients and genes differentially regulated at a false significant rate of <5% (as determined by SAM). Results are displayed with Treeview software (Eisen, et al., referenced above).

Results

Transcription profiling confirms the importance of recognized markers of left ventricular dysfunction. cDNA derived from paired samples of human left ventricle, harvested at the time of LVAD implantation and later at the time of cardiac transplantation, was hybridized to cDNA microarrays containing 13,302 features (Agilent Technologies, Palo Alto, California). Eleven patients were included in the study (Table 1). The number of genes significantly upregulated after mechanical offloading was greater than the number downregulated (FIG. 1A). Table 2A presents names, Genbank accession numbers, rank consistency scores, SAM scores and ranks, and median fold change from pre to post LVAD for genes that were upregulated after implantation of an LVAD. Table 3 presents the same information for genes that were downregulated after implantation of an LVAD. Genes with reduced message included those coding known markers or marker-precursors of heart failure such as natriuretic peptide precursor A (Unigene Hs.75640) and natriuretic peptide precursor B (Hs.219140). In addition, the natriuretic peptide features clustered together when subjected to average-linkage hierarchical clustering (FIG. 1B). These findings provide a unique validation for the use of natriuretic peptides as clinical markers, through their emergence from a screening pool of many thousand genes. Additionally, they provide a rationale for the use of paired samples from offloaded hearts to characterize the genetic profile of heart failure.

Identification of genes not previously recognized as important in heart failure. The list of genes differentially regulated from pre to post LVAD contains genes previously unrecognized to be important in heart failure. Mitogen-activated protein kinase 4 (MAPK-4, Hs.269222, also called ERK3, ERK4 and p63MAPK) was first cloned in 1992 and is a distantly related member of the mitogen-activated protein kinase family of serine/threonine kinases. It was highly and consistently downregulated post-LVAD, ranked ahead of all other genes by the Significance Analysis of Microarrays (SAM, FIG. 1A) and 10th on the rank consistency table of downregulated genes (Table 2A). The SAM (17) ranks genes by a t statistic, which emphasizes overall pre to post differences per gene as a function of variance, controlling the error rate by a permutation procedure. In contrast, the rank consistency score more directly rewards consistency of change for a given gene across many individuals, offsetting the effect of individual variation.

Downregulation of a splice variant of the regulatory domain (alpha subunit) of the L-type calcium channel following offloading (AF233289, ranked 3rd by SAM) is relevant, given that changes in calcium dynamics are a central component of heart failure pathogenesis. Although the role of the myosin light chain pseudogene (AF042089, 6th in rank consistency) remains unknown, myosin light chain kinase itself is a key mediator of sarcomeric organization in cardiac hypertrophy (Aoki, H., Sadoshima, J. & Izumo, S. Myosin light chain kinase mediates sarcomere organization during cardiac hypertrophy in vitro. Nat Med 6, 183-8 (2000)). The results described herein point to a possible role of this related gene in heart failure. Myosin light chain 2a itself (W17098, 5th in rank consistency) is a highly conserved and early marker of atrial chamber differentiation in organogenesis, but is found here in the ventricle, suggesting a role for this gene in left ventricular hypertrophy and failure.

Several immunological markers, such as interleukins, interferons, tumor necrosis factor related genes and major histocompatibility complex genes are upregulated post LVAD. Hierarchical average-linkage clustering (FIG. 1B) groups these genes together suggesting a coordinated immune response to the implantation of a prosthesis in the thorax.

Expression of the APJ Receptor is Markedly Elevated Post LVAD

Two distinct statistical analyses separately identified APJ (angiotensin receptor-like 1, Hs.9305) as the gene most significantly and consistently upregulated following LVAD implantation (FIG. 1A, Table 2A). The SAM score (4.872) was greater than all others, while pre to post LVAD fold change was estimated by hybridization at 3.2 and by quantitative real time PCR at 4.12. These relatively conservative fold changes contrast with the magnitude of the SAM score and emphasize the importance of variance in the analysis of microarray data.

TABLE 1
Clinical characteristics of patients undergoing LVAD implantation
and cardiac transplantation.

| Age, y/Gender | Diagnosis | Duration, d | qRT-PCR |
|---|---|---|---|
| 63/M | ICM | 170 | + |
| 17/M | IDCM | 36 | + |
| 48/M | ICM | 28 | + |
| 31/M | IDCM | 14 | |
| 46/M | IDCM | 158 | |
| 48/M | IDCM | 175 | + |
| 55/M | ICM | 323 | |
| 54/M | ICM | 36 | + |
| 30/M | HCM | 89 | + |
| 46/M | GCCM | 85 | |
| 18/M | IDCM | 92 | + |

TABLE 2A
Genes significantly upregulated following implantation of a left ventricular assist device.

| | | Rank | | | Median |
|---|---|---|---|---|---|
| Accession | | consistency | SAM | SAM | fold |
| number | GeneName | p-value | score | rank | change |
| U03642 | angiotensin receptor-like 1 | 3.57183E−11 | 4.87 | 1 | 3.34 |
| M55542 | guanylate binding protein 1, interferon- | 1.00574E−09 | 3.25 | 26 | 3.00 |
| | inducible, 67 kD | | | | |
| NM_004772 | P311 protein | 1.00574E−09 | 3.49 | 12 | 2.66 |
| X03100 | major histocompatibility complex, class II, | 1.85962E−08 | 3.39 | 20 | 2.46 |
| | DP alpha 1 | | | | |
| W17098 | myosin light chain 2a | 8.29759E−08 | 2.25 | 211 | 2.36 |
| AF042089 | myosin light chain kinase pseudogene | 3.72431E−07 | 3.82 | 6 | 2.14 |
| M24895 | amylase, alpha 2B; pancreatic | 6.34838E−07 | 2.37 | 162 | 3.21 |
| AJ006945 | purinergic receptor P2Y, G-protein | 6.64978E−07 | 3.43 | 17 | 1.80 |
| | coupled, 1 | | | | |
| AL137572 | niban protein | 1.56273E−06 | 3.01 | 48 | 1.81 |
| M30682 | Homo sapiens beta-2-microglobulin | 1.64434E−06 | 3.38 | 21 | 1.77 |
| NM_001548 | interferon-induced protein with | 2.13023E−06 | 3.04 | 44 | 1.86 |
| | tetratricopeptide repeats 1 | | | | |
| X00457 | major histocompatibility complex, class II, | 2.15898E−06 | 3.21 | 29 | 2.16 |
| | DP alpha 1 | | | | |
| NM_{——}004961 | gamma-aminobutyric acid (GABA) A | 2.22739E−06 | 1.75 | 593 | 1.79 |
| | receptor, epsilon | | | | |
| AF092922 | retinoic acid receptor responder (tazarotene | 2.31815E−06 | 3.90 | 5 | 1.83 |
| | induced)3 | | | | |
| M33882 | myxovirus (influenza) resistance 1 | 2.44421E−06 | 2.63 | 96 | 2.19 |
| M21121 | small inducible cytokine A5 (RANTES) | 4.24897E−06 | 3.61 | 8 | 1.52 |
| AL050366 | O-linked N-acetylglucosamine (GlcNAc) | 4.5E−06 | 3.03 | 45 | 1.81 |
| | transferase | | | | |
| M24097 | major histocompatibility complex, class I, C | 4.74467E−06 | 2.94 | 56 | 1.67 |
| AW103366 | ubiquinol-cytochrome c reductase binding | 8.15774E−06 | 2.33 | 178 | 1.59 |
| | protein | | | | |
| AA034349 | hypothetical protein, expressed in | 8.21995E−06 | 2.84 | 68 | 1.75 |
| | osteoblast | | | | |
| AI272713 | 2′,5′-oligoadenylate synthetase 1 | 1.02906E−05 | 2.28 | 200 | 1.67 |
| D49742 | hyaluronan-binding protein 2 | 1.10354E−05 | 1.59 | 796 | 2.32 |
| AP002534 | Human genomic DNA, chromosome 1q22-q23, | 1.20871E−05 | 0.97 | 2140 | 1.58 |
| | CD1 region, section ¾. | | | | |
| M57399 | pleiotrophin (heparin binding growth factor | 1.22633E−05 | 2.62 | 101 | 1.70 |
| | 8, neurite growth-promoting factor 1) | | | | |
| X82321 | peroxiredoxin 2 | 1.28978E−05 | 3.11 | 36 | 1.71 |
| U37518 | tumor necrosis factor (ligand) superfamily, | 1.45601E−05 | 2.69 | 87 | 1.90 |
| | member 10 | | | | |
| Y00081 | interlueukin 6 (interferon, beta 2) | 1.54052E−05 | 4.16 | 4 | 1.60 |
| AAA40703 | Agrin | 1.64071E−05 | 1.31 | 1285 | 2.28 |
| AF088219 | small inducible cytokine subfamily A | 1.71064E−05 | 3.49 | 13 | 1.85 |
| | (Cys-Cys), member 15 | | | | |
| X03663 | colony stimulating factor 1 receptor | 1.897E−05 | 3.14 | 34 | 1.64 |
| AA313375 | H3 histone, family 3A | 2.05168E−05 | 4.66 | 2 | 2.15 |
| NM_001343 | disabled (Drosophila) homolog 2 (mitogen- | 2.24728E−05 | 3.36 | 24 | 2.20 |
| | responsive | | | | |
| | phosphoprotein) | | | | |
| AK000066 | hypothetical protein FLJ20059 | 2.35498E−05 | 2.59 | 106 | 1.53 |
| U90548 | butyrophilin, subfamily 3, member A3 | 2.4105E−05 | 2.46 | 135 | 1.66 |
| X70683 | SRY (sex determining region Y)-box 4- | 2.72346E−05 | 4.34 | 3 | 1.70 |
| U04245 | major histocompatibility complex, class I, B | 3.08133E−05 | 2.92 | 57 | 1.54 |
| N20599 | cathepsin O | 3.09127E−05 | 3.36 | 22 | 1.92 |
| L11910 | retinoblastoma 1 (including osteosarcoma) | 3.11125E−05 | 3.46 | 15 | 1.44 |
| AX011749 | Sequence 151 from Patent WO9955858 | 3.22318E−05 | 2.26 | 208 | 1.67 |

TABLE 2B
Comparison of Apelin and APJ in Human, Mouse, and Rat

| | Apelin | | | APJ | | |
|---|---|---|---|---|---|---|
| Species | Human | Mouse | Rat | Human | Mouse | Rat |
| Protein | 100% | 83% | 81% | 100% | 92% | 89% |
| similarity | | | | | | |
| Chromosome | X | X | X | 11 | 2 | 3 |
| No. of Exons | 3 | 3 | 3 | 2 | 1 | 10 |
| Gene length | 9127 bp | 9917 bp | 7697 kp | 3658 bp | 1134 bp | 2.28 kp |
| Transcript | 2673 bp | 3071 bp | 1287 bp | 1726 bp | 1134 bp | 1898 bp |
| length | | | | | | |
| Coding | 233 bp | 234 bp | 235 bp | 1140 bp | 1131 bp | 591 bp |
| sequence | | | | | | |
| Translation | 77 aa | 77 aa | 77 aa | 380 aa | 377 aa | 197 aa |
| length | | | | | | |

TABLE 3
Genes significantly downregulated following implantation of a left ventricular assist device.

| | | Rank | | | Median |
|---|---|---|---|---|---|
| Accession | | consistency | SAM | SAM | fold |
| number | GeneName | p-value | score | rank | change |
| M25296 | natriuretic peptide precursor B | 3.97174E−20 | −4.02 | 5 | 11.01 |
| NM_004317 | arsA (bacterial) arsenite transporter, ATP- | 7.15531E−12 | −4.56 | 2 | 2.53 |
| | binding, homolog 1 | | | | |
| AB037521 | Human gene for natriuretic protein, partial | 8.61857E−10 | −4.12 | 4 | 19.23 |
| | cds. | | | | |
| D29767 | tec protein tyrosine kinase | 1.26151E−09 | −3.02 | 20 | 2.72 |
| M69238 | aryl hydrocarbon receptor nuclear | 9.47527E−09 | −2.64 | 48 | 2.21 |
| | translocator | | | | |
| U42408 | ladinin 1 | 1.1674E−08 | 3.65 | 9 | 1.88 |
| AW843848 | phospholipase A2, group IIA (platelets, | 3.70971E−08 | −2.40 | 89 | 4.06 |
| | synovial fluid) | | | | |
| NM_001908 | cathepsin B | 5.35531E−08 | −2.39 | 93 | 2.33 |
| NM_001809 | centromere protein A (17 kD) | 7.76821E−08 | −1.82 | 343 | 1.93 |
| X59727 | mitogen-activated protein kinase 4 | 9.76292E−08 | −5.57 | 1 | 2.87 |
| NM_005928 | milk fat globule-EGF factor 8 protein | 2.28804E−07 | −3.15 | 18 | 1.92 |
| D38047 | proteasome (prosome, macropain) 26S | 3.66325E−07 | −3.87 | 7 | 2.03 |
| | subunit, non-ATPase, 8 | | | | |
| X69910 | transmembrane protein (63 kD) | 4.31941E−06 | −2.57 | 56 | 1.55 |
| AF125533 | cytochrome b5 reductase 1 (B5R.1) | 4.70629E−06 | −2.77 | 37 | 1.70 |
| AF060567 | sushi-repeat protein | 8.66718E−06 | −2.52 | 67 | 1.85 |
| U97276 | quiescin Q6 | 1.11165E−05 | −1.71 | 450 | 1.64 |
| NM_005637 | synovial sarcoma, translocated to X | 1.21749E−05 | −4.01 | 6 | 1.94 |
| | chromosome | | | | |

### EXAMPLE 2

### Confirmation of Microarray Hybridization Studies by Quantitative Real-Time PCR

Materials and Methods

Quantitative real-time RT-PCR. Five genes were assessed in seven individuals using quantitative real time RT-PCR on the ABI PRISM® 7900HT Sequence Detection System (TaqMan, Applied Biosystems, Foster City, Calif.). Primers and probes were obtained from Applied Biosystems' Assays-on-Demand™. After DNase treatment, cDNA was synthesized from 5 μg of RNA using MMLV reverse transcriptase (SuperScript II kit, Invitrogen, Carlsbad, Calif.). Amplification was carried out in triplicate: 50° C. for 2 min, 95° C. for 10 min, followed by 40 cycles of 95° C. for 15 sec and 60 ° C. for 1 min. A standard curve derived from TNFα-stimulated human aortic endothelial cell RNA was plotted for each target gene. RNA quantity was expressed relative to 18S endogenous control. Fold differences were calculated by dividing the post LVAD sample by the pre LVAD sample. Linear regression was carried out using SPSS version 11.0.

Results

Although use of a reference RNA controls much of the variation introduced by the dynamics of hybridization, the gold standard for quantitation of mRNA remains quantitative real time polymerase chain reaction (qRT-PCR, Taqman). qRT-PCR was performed for 5 of the most differentially regulated genes in both samples of 7 individuals chosen at random, expressing the post-LVAD value as a ratio of that pre-LVAD for each individual and gene. The 5 genes were: angiotensin receptor-like 1 (APJ), interleukin 6, mitogen-activated protein kinase 4, atrial natriuretic peptide, and brain natriuretic peptide. A close relationship was found between the magnitude of change as measured by hybridization and qRT-PCR (FIG. 1D; y=0.49×, R2=0.86, P<0.0001) confirming previous authors' observations (Barrans, J. D., Allen, P. D., Stamatiou, D., Dzau, V. J. & Liew, C. C. Global gene expression profiling of end-stage dilated cardiomyopathy using a human cardiovascular-based cDNA microarray. Am J Pathol 160, 2035-43 (2002)) and suggesting that ratiometric hybridization accurately reflects gene expression across the array.

### EXAMPLE 3

### Measurement of Apelin Levels in Cardiac Tissue

Materials and Methods

Apelin assay. Eight mg of tissue was boiled in 0.1 M acetic acid for 10 minutes, homogenized, then centrifuged at 12,000 rpm for 10 minutes and the supernatant used to quantify total protein concentration via the Bradford Assay (Biorad, Hercules, Calif.). Equal amounts of total protein (concentration 300 μg/ml) were used in the Apelin-12 EIA assay kit (Phoenix Pharmaceuticals, Belmont, Calif.) following manufacturer's instructions. 50 μl of plasma was used directly for the assay. Comparisons were made using Student's paired t-test and one way analysis of variance with post hoc tests according to Fisher (SPSS software version 11.0).

Results

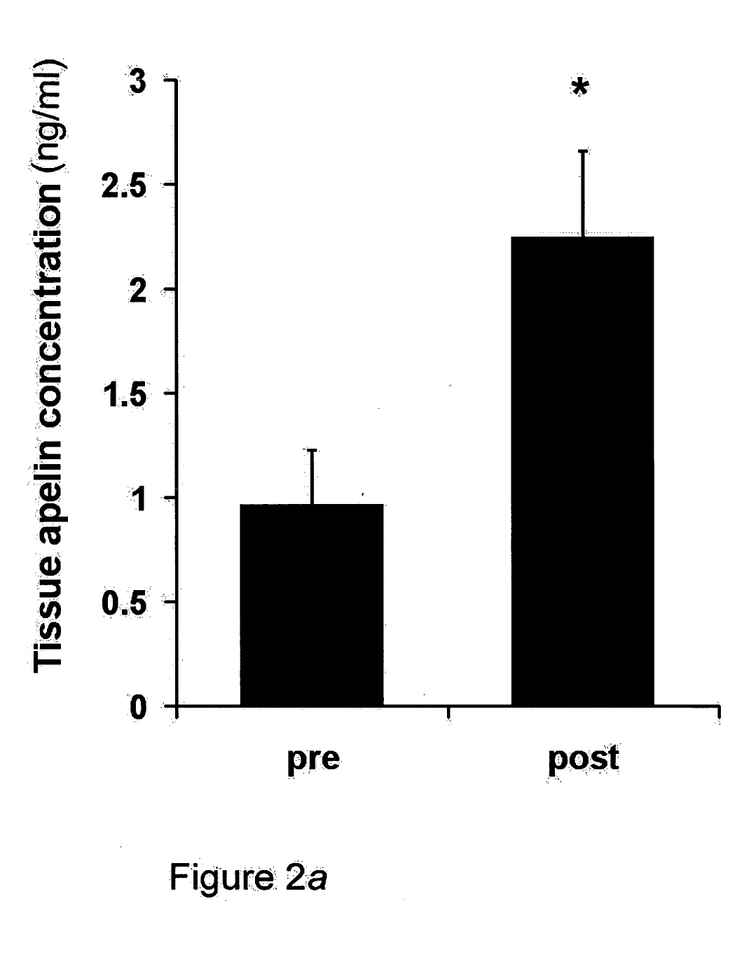

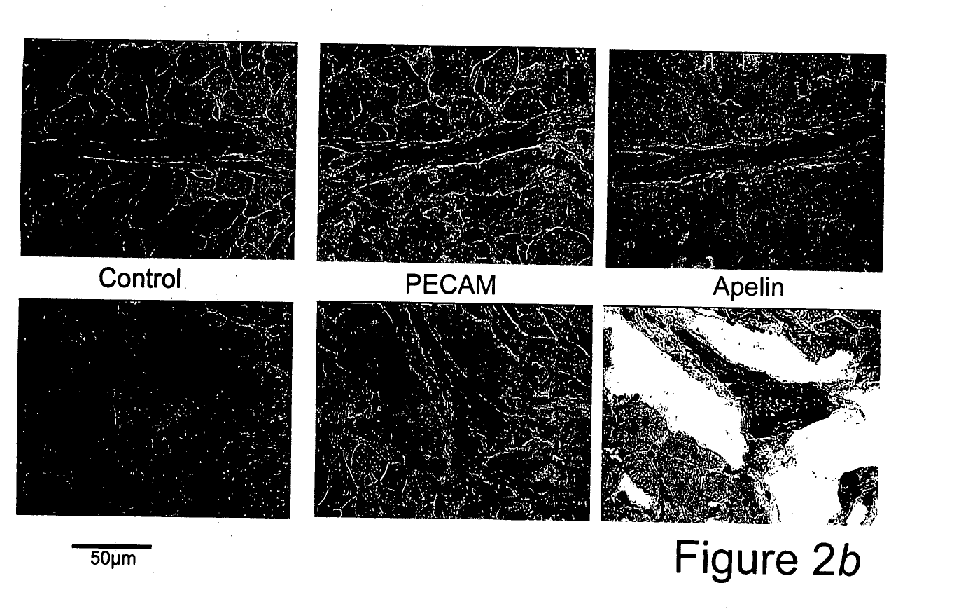

Apelin is increased in cardiac tissue following LVAD implantation. Competitive enzyme immunoassay was used to detect levels of apelin in the samples of left ventricle that were used for hybridization. Tissue apelin levels were significantly higher post LVAD (FIG. 2A; pre 0.967±0.26; post 2.246±0.41 in ng/ml; P<0.001; units are concentration of apelin in ng/ml within a normalized total protein concentration of 300 μg/ml). This reflects a change in the upward direction in all but two patients. Since the expression of the receptor and its ligand were moving in concert, we examined the relationship between the two per individual. A weak but significant positive correlation was found (y=−0.42+1.15×; R2=0.3; P=0.019; data not shown).

### EXAMPLE 4

### Localization of Apelin in Human Cardiac Tissue

Materials and Methods

Immmunohistochemistry. Tissue was frozen in OCT (Tissue-Tek, Torrance, Calif.). Four micron thick sections were cut and stored at minus 80° C. Slides were fixed in minus 20° C. acetone, and air dried. Blocking was achieved using 10% goat serum (Zymed, S. San Francisco, Calif.). Sections were stained with apelin polyclonal antibody (Phoenix Pharmaceuticals, Belmont, Calif.) and with CD31 (Cymbus Biotechnology Ltd, England) Secondary incubation used anti-rabbit envision+(Dako, Carpenteria, Calif.) for apelin and anti-mouse envision+(Dako, Carpenteria, Calif.) for CD31. The chromagen substrate 3-amino-9-ethylcarbazole was used. Sections were counterstained using hematoxylin.

Results

Apelin is highly specifically localized to the vasculature in cardiac tissue. Immunohistochemistry was carried out with the same antibody used for detecting apelin tissue levels by enzyme immunoassay. The localization of apelin in normal human cardiac left ventricle was compared with that from end stage, failing left ventricle. In both tissues, the distribution of staining was similar. We found that cardiac vessels stain densely for apelin with negligible staining in myocardial cells (FIG. 2B). Staining of consecutive sections for PECAM (CD31) confirmed the endothelial localization, although high powered views suggested apelin staining extended to smooth muscle cells also. Despite little staining overall of the myocardium, in the failing heart, apelin was detectable at low levels in the myocardial cells also suggesting extension of the signaling system in late stage disease (data not shown).

### EXAMPLE 5

### Measurement of Plasma Apelin Levels in Humans with Heart Failure

Materials and Methods

Apelin assay. This was performed as described in Example 3, but rather than tissue, 50 μl of plasma was used.

Results

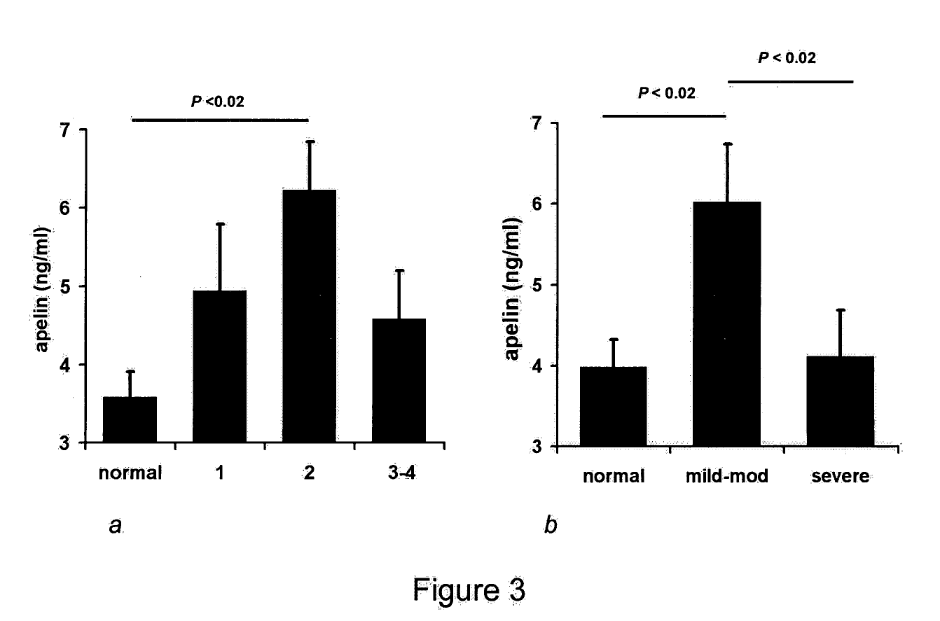

To determine the role of apelin in earlier stages of heart failure, plasma levels of apelin were measured in blood from 80 heart failure patients with a broad spectrum of disease severity (male n=63, female n=17, mean age 63 years, standard deviation 10 years). Since plasma apelin levels had not previously been reported in humans, 32 normal subjects were also studied to determine the range of normal. Plasma apelin was detectable in plasma from healthy human subjects (3.58±0.33 ng/ml), rose in the early stages of heart failure (New York Heart Association class 1:4.94±0.85 ng/ml) and was maximum in those classified as NYHA Class 2 (6.22±0.63, P<0.02). In those with severe disease, plasma apelin was lower (NYHA Class 3-4: 4.58±0.62 ng/ml) but this change was not significant (FIG. 3A). Mirroring the changes in functional class, dividing the patients by ejection fraction also revealed a rise in apelin from normal to mild-to-moderate LV dysfunction (3.98±0.34 vs 6.02±0.72 ng/ml, P<0.02). Similarly, in later stage disease, apelin level declined (severe LV dysfunction 4.11±0.58 ng/ml, P<0.02, FIG. 3B).

### EXAMPLE 6

### Localization of Apelin and APJ in Developing and Adult Mouse Heart

Materials and Methods

Expression of the APJ receptor. Adult mice were perfusion fixed with 4% paraformaldehyde, and adult heart and whole embryos further fixed over night, embedded in paraffin and sectioned. Five micron thick sections were cut and stored at 4° C. Blocking was achieved using 1.5% goat serum (Vector labs, Burlingame, Calif.). Sections were stained with APJ polyclonal antibody (Lifespan Biosciences, Seattle, Wash., LSA64, 1/100 dilution). Secondary antibody was biotinylated anti-rabbit raised in goat (Vector labs, Burlingame, Calif., 1/200 dilution). For embryo sections, the chromagen substrate was BCIP/NBT for alkaline phosphatase (Vector labs, Burlingame, Calif.), and sections were counterstained with nuclear fast red. For the heart sections, the chromagen substrate was vector red for alkaline phosphatase (Vector labs, Burlingame, Calif.), and sections were counterstained with hematoxylin.

Results

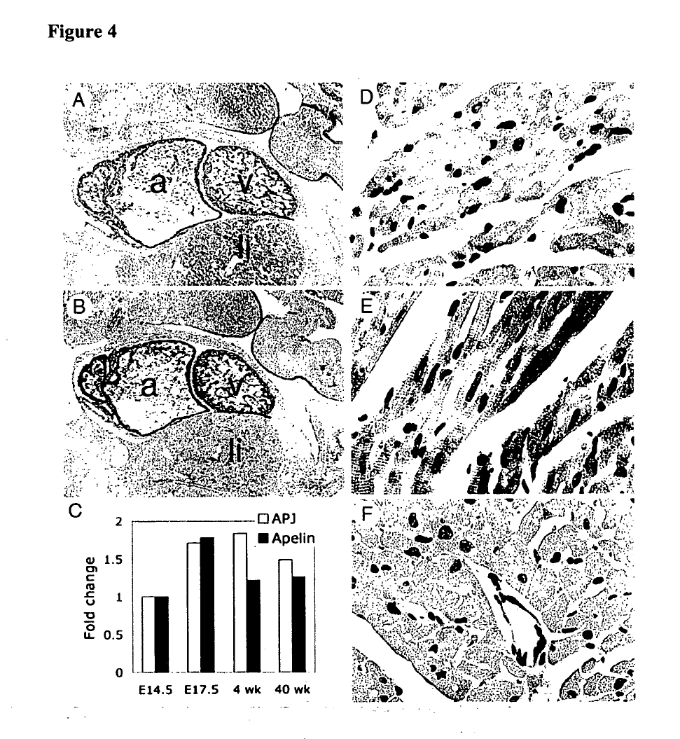

Expression of APJ receptor. Immunohistochemistry with antibodies for apelin and APJ revealed specific immunolocalization of both proteins in the developing myocardium, with very similar patterns of expression as early as embryonic day 13.5 (FIG. 4, Panels A, B). Real time quantitative RT-PCR of isolated heart mRNA validated this finding, and suggested that the relative contribution to the total myocardial RNA by these transcripts remains relatively constant through late gestation and adulthood (FIG. 4, Panel C). In the adult mouse heart, immunolocalization of APJ expression was identified in association with both atrial and ventricular myocardial cells (FIG. 4, Panels D-F).

### EXAMPLE 7

### Effects of Acute and Chronic Apelin Administration In Vivo

Materials and Methods

Peptide reagents. Apelin-12 was purchased from Bachem (Bachem Bioscience, King of Prussia, Pa.). Pyroglutamylated apelin-13 (PYR-apelin-13) was purchased from American Peptide Company (Sunnyvale, Calif.). Apelin-12 circulates as pyroglutamylated apelin 13 and the latter is felt to be more stable. Apelin was dissolved in distilled, autoclaved, degassed water, frozen at −20 degrees C at high concentration, and aliquoted on the morning of use.

Magnetic resonance imaging. Male C57B/16 mice aged 16 weeks (n=9) were scanned twice on subsequent days. The animals underwent general anesthesia while breathing spontaneously via a nose cone fitted carefully to minimize escape of anesthetic into the environment. 2% isoflurane was administered with an oxygen flow rate of 1-2 l/min. Platinum needle ECG leads were inserted subcutaneously. Respiration was monitored by means of a pneumatic pillow sensor positioned against the abdomen. Mouse body temperature was maintained during scanning at 37° C. by a flow of heated air thermostatically controlled by a rectal temperature probe. Magnetic resonance images were acquired on a 4.7T Oxford magnet controlled by a Varian Inova console (Varian, Palo Alto, Calif.) using a transmit-receive, quadrature, volume coil with an inner diameter of 3.5cm. Image acquisition was gated to respiration and to the ECG R wave (SA Instruments, Stony Brook, N.Y.). Coronal and sagittal scout images led to the acquisition of multiple contiguous lmm thick, short axis slices orthogonal to the interventricular septum. Nine cine frames were taken at each slice level with the following sequence parameters: TE=2.8ms, NEX=12, FOV=3×3 cm, matrix=128×128, flip angle=60°. Cine frames were spaced 16 ms apart and acquired through slightly more than one cardiac cycle guaranteeing acquisition of systole and diastole. On the second day of scanning, mice received 300 μg/kg body weight of apelin-12 as an intraperitoneal injection one hour prior to scanning. A pilot study had previously identified one hour as an appropriate time within which to identify apelin effects resulting from peritoneal absorption. Planimetry measurements of end diastolic and end systolic dimension were derived offline from short axis views of the left ventricle at the level of the papillary muscles using ImageJ software (National Institutes of Health, Bethesda, Md.). Ejection fraction was calculated as [LVEDA-LVESA]/LVEDA.

Pressure-volume hemodynamics. Pressure-volume hemodynamics were assessed using the Aria System (Millar Instruments, Houston, Tex.). Male C57B1/6 mice aged 8-12 weeks (n=10) were anesthetized with 1-2% isoflurane in oxygen. The internal jugular vein was cannulated with PE tubing and a 10% albumin solution infused at 5 μl/min following a bolus of 150 μl over 5 minutes. After tracheotomy, a 19G cannula was inserted into the trachea and the animal was ventilated at a tidal volume of 200 μl at 100 breaths per minute (Harvard Apparatus, Holliston, Mass.). Mice were warmed throughout the procedure and constantly monitored for depth of anesthesia. Following an incision just dorsal to the xyphoid cartilage, the diaphragm was visualized from below, and after diaphragmatic incision, the left ventricular apex was visualized. The pressure-volume catheter was then inserted along the long axis of the left ventricle, from where it was adjusted to obtain rectangular shaped pressure-volume loops. Appropriate position was verified post mortem (FIG. 2, Panel D). Baseline loops were recorded following volume replacement, at which point, the inferior vena cava was visualized within the chest and occlusion parameters were recorded during and after a 5 second manual occlusion of this vessel. Next, the albumin solution was replaced by one containing 100 nM Apelin-12 which was infused at 5 μl/min for 20 minutes, following which, baseline and occlusion loops were recorded once again. Signals from the catheter were digitized using the Powerlab system (ADInstruments, Colorado Springs, Colo.) and stored for offline analysis using the PVAN software (Pressure-volume ANalysis, Millar Instruments, Houston, Tex.).

Chronic apelin infusion. To test longer term effects of apelin, we infused 2 mg/kg/day PYR-apelin-13 into 8-12 week male C57/B1/6 mice. A short anesthetic (isoflurane 1% in oxygen 11/min) facilitated implantation of a 2 ml osmotic minipump under the scruff with staple closure (Alzet Osmotic pumps, Cupertino, Calif., model 1002). Minipumps contained either PYR-apelin-13 (n=10) or sterile normal saline (n=5). Cardiovascular parameters were recorded at 7 days and 14 days by tail cuff sphygmomanomtery (Visitech Systems, Apex, N.C.) and echocardiography (probe frequency 15 MHz, Acuson Sequoia, Siemens, Malvern, Pa.). For echocardiography, mice were anesthetized using isoflurane (0.75-1.25% in oxygen 11/min) then placed supine and warmed using a heat lamp. Using a gel buffer, parastemal long and short axis views were recorded in each animal to allow estimation of indices of contractility such as fractional shortening (LVEDD-LVESD/LVEDD), cardiac output ([Pi*(Aod)\hat{ }2*VTI*HR]/4), velocity of circumferential shortening ([LVEDD−LVESD]/[ET×LVEDD]), and LV mass (1.05*[(IVSD+LVEDD+PWTD)³−LVEDD³) where LVEDD is left ventricular end diastolic diameter, LVESD is left ventricular end systolic diameter, IVSD is inter ventricular septum in diastole, PWTD is posterior wall thickness in diastole, Aod is aortic diameter, VTI is the velocity time integral, ET is ejection time. The last two parameters are derived from Doppler sampling of the outflow tract. All measurements were made by one operator blinded to group. At 7 and 14 days of infusion, these measurements were repeated. Mice were then sacrificed and organs removed for measurement of wet weight.

Data Analysis

Data were analyzed using Student's t statistic (paired) or the repeated measures analysis of variance using the post hoc comparison of Fisher (NCSS 2002). Exact p values are reported for all comparisons.

Results

Magnetic resonance imaging. Contractility in C57B1/6 mice was first assessed at baseline. Short axis views of the left ventricle at the level of the papillary muscles allowed estimation of end systolic and end diastolic areas by planimetry (FIG. 5, Panel D). Apelin had no effect on the spontaneous rate of respiration (pre: 68±11; post: 66±7 breaths per minute, p=0.7) while heart rate calculated as the inverse of the R—R interval of the electrocardiogram was significantly greater (pre: 537±20; post 559±19 beats per minute, p=0.03, FIG. 5, Panel A). Ejection fraction tended to increase following apelin injection but this did not reach significance (pre: 63.6±2.7; post 67.7±1.6 %, p=0.16, FIG. 5, Panel B). However, the end diastolic area was very significantly reduced following apelin injection (pre: 0.122±0.007; post: 0.104±0.005 cm2, p=0.006, FIG. 5, Panel C).

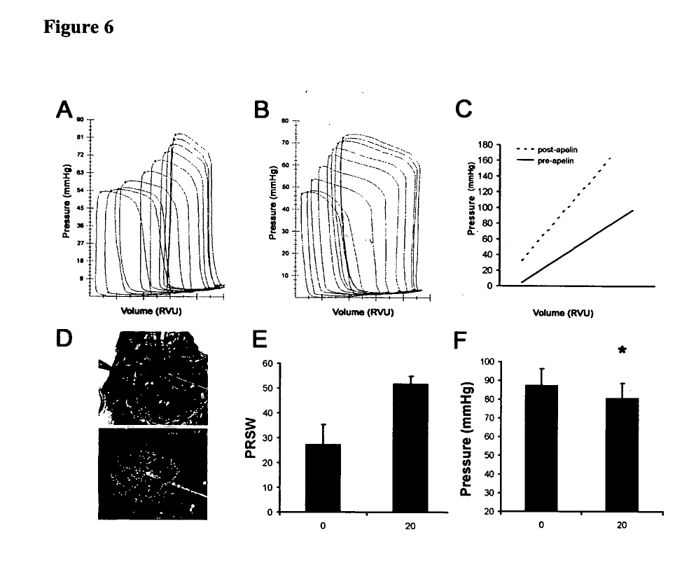

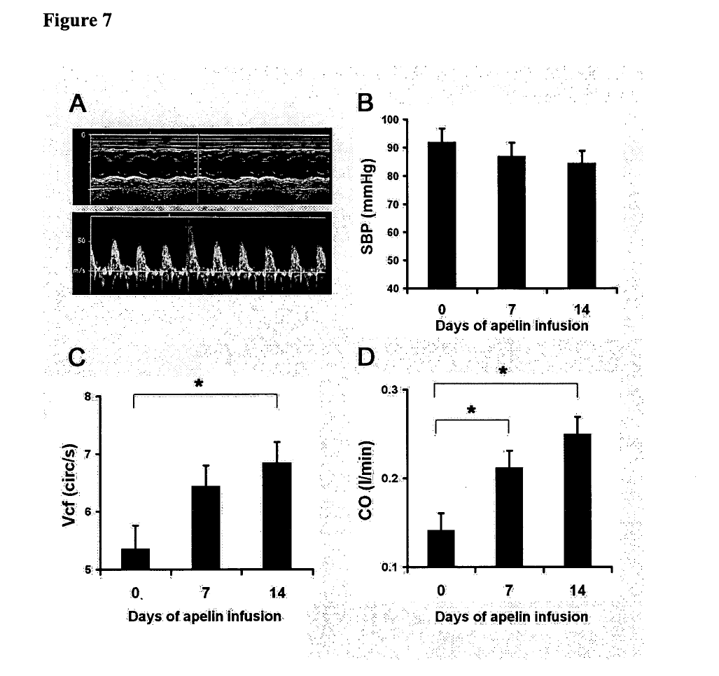

Pressure-volume hemodynamics. Separating effects of load and function is not possible with non-invasive imaging and since apelin has effects on both vascular reactivity and intrinsic contractility, we elected to assess ventriculo-vascular coupling via pressure-volume hemodynamics (Table 4, FIG. 6). Here, intraventricular pressure is measured directly while intraventricular volume is estimated by conductance. Thus, effects of load and intrinsic contractility can be simultaneously and independently assessed through the construction of pressure-volume loops, both at baseline and during preload reduction, achieved by manual compression of the inferior vena cava. Baseline measurements were consistent with those previously reported in the literature for the C57B1/6 mouse (Table 4). Thoracotomy requires greater depth of anesthesia than non-invasive imaging and this contributes to a lower heart rate in invasive studies. This mild but obligatory cardiovascular depression may also explain the lack of increase in heart rate (pre: 376±21; post: 353±33 bpm, p=0.3) which was seen in the MRI studies.

Systolicfunction. Consistent with the MR data, LV end diastolic volume was lower, but this difference was not significant (pre: 29.3±6.1; post: 26.2±5.9 RVU, p=0.4). Similarly, load dependent measures of contractility such as ejection fraction and dP/dtmax did not change following apelin infusion (Table 4). However, inspection of occlusion parameters revealed significant changes in load independent measures of contractility. Both the slope and intercept of the end systolic pressure-volume relationship were increased following apelin infusion (FIG. 6, Panels A-C). Corresponding to this finding, the time varying elastance (the maximum slope of a series of lines drawn through each point in the cardiac cycle) was significantly greater (pre: 6.0±1.5; post: 12.7±3.1, p=0.017). There were also increases in pre-load recruitable stroke work (a regression line fitted to the relationship between end diastolic volume and stroke work; stroke work representing the area of the pressure-volume loop, FIG. 6, Panel E). Although, dP/dtₘₐₓ itself was not different following apelin infusion, the relationship between dP/dtₘₐₓ and end diastolic volume was steeper and its intercept greater (Table 4). Arterial elastance, a steady-state parameter that incorporates peripheral resistance, impedance, compliance and systolic/diastolic time intervals (approximated by the steady state LV end systolic pressure to stroke volume ratio) did not change significantly, however the maximum developed pressure (FIG. 6, Panel F) and end systolic pressure were reduced, most likely reflecting a lower arterial pressure and earlier opening of the aortic valve (confirmed by a lower pressure at dP/dtₘₐₓ).

Diastolic function. Time constants of relaxation were not different after apelin infusion (data not shown). In addition, the slope and intercept derived from a linear model fit of the end diastolic pressure-volume relationship were not different. However, pressure decay is known to be load dependent and model dependency of diastolic parameters is well recognized (Kass D A. Assessment of diastolic dysfunction. Invasive modalities. Cardiol Clin., 18:571-86, 2000). When the end diastolic pressure-volume relationship was fit by a monoexponential of the form [LVEDP=k1*exp(k2*LVEDV)], the constant k1 was greater (pre: 0.064±0.008; post: 0.111±0.002, p=0.09) while the exponential k2 was unchanged (pre: 0.83±0.22; post:0.69±0.41, p=0.66).

Chronic apelin infusion. We infused PYR-apelin-13 over the course of two weeks at a level previously shown to exert acute hemodynamic effects. No significant changes were seen in saline infused controls from baseline to 14 days. Significant changes in heart rate and blood pressure, known to occur acutely, were not detected over the period of the chronic apelin infusion: neither conscious heart rates (tail cuff method) nor isoflurane heart rates (echo Doppler) were different at any time point (data not shown) and while systolic blood pressure (SBP) trended lower during apelin infusion this change was not significant (Figure, Panel 7B). Left ventricular contractility measurements derived from Doppler ultrasound of the left ventricular aortic outflow tract were significantly increased during apelin infusion. The velocity of circumferential shortening was increased at 14 days (p=0.049, Panel 7C). Similarly, cardiac output was increased at 7 days and this increase was maintained at 14 days (p=0.001, Panel 7D). Despite these increases in contractility, post mortem organ weights were not different between the saline and apelin groups.

TABLE 4
Change in invasive hemodynamic indices following acute apelin infusion.

| | HR | | EDV | | Pmax | | EF | | Ees | | intercept | |
|---|---|---|---|---|---|---|---|---|---|---|---|---|
| | mean | sem | mean | sem | mean | sem | mean | sem | mean | sem | mean | sem |
| Pre apelin | 376 | 21 | 29.3 | 6.1 | 87.5 | 8.9 | 61.2 | 6.8 | 3.7 | 0.9 | −14.2 | 5.9 |
| Post apelin | 353 | 33 | 26.2 | 5.9 | 80.7 | 7.9 | 59.1 | 9 | 6.5 | 1.4 | 0.5 | 5.3 |
| p value | 0.3 | | 0.4 | | 0.02 | | 0.8 | | 0.018 | | 0.013 | |

| | PRSW | | intercept | | dPdt-EDV | | intercept | | Emax | |
|---|---|---|---|---|---|---|---|---|---|---|
| | mean | sem | mean | sem | mean | sem | mean | sem | mean | sem |
| Pre apelin | 27.4 | 8.0 | −41.8 | 21.3 | 127.5 | 27.3 | −22.2 | 5.1 | 6 | 1.5 |
| Post apelin | 51.8 | 3.1 | 11.3 | 3.1 | 179.1 | 34.1 | −2.3 | 4.8 | 12.7 | 3.1 |
| p value | 0.059 | | 0.045 | | 0.16 | | 0.04 | | 0.017 | |

### EXAMPLE 8

### Apelin Treatment Improves Exercise Capacity in Mice with Heart Failure

Materials and Methods

Left anterior decending artery ligation. Animals were anesthetized with 2-3% inhalational isofluorane and 50 mg/kg sodium pentobarbital intraperitoneally. They were intubated with a 14 gauge angiocath and positive pressure ventilation with an oxygen/isofluorane mixture was achieved with the Harvard Rodent ventilator. The animals were placed in the left lateral decubitus position and a thoracotomy performed at the 4th intercostal space. Peak inspiratory pressures were monitored and maintained between 10-14 cm of water. The lung was retracted and the pericardium incised. The left coronary artery was ligated with a 7-0 prolene suture until blanching of the distal left ventricle was noted. Heart rate and temperature were monitored during the procedure. After ascertaining complete hemostasis, the chest wall was closed in four layers. The animals were weaned from the ventilator and anesthetic and then extubated and monitored in the recovery area.

Osmotic minipump. Animals were allowed to recover over 4 weeks and developed moderate heart failure over this time. At this point, an osmotic minipump containing apelin was inserted as described in Example 7.

Treadmill exercise. Treadmill exercise has long been the gold standard for inducing controlled cardiovascular stress in humans and other large mammals. In mice, a commonly used commercially available setup (Columbus Instruments) employs a moving belt encased in a sealed Plexiglas enclosure, allowing for measurement of oxygen consumption and carbon dioxide production. Stimulus devices consist of a metal shock grid. Metabolic measurements are performed using an open circuit volumetric gas analysis system (Oxymax System; Columbus Instruments). The low dead space of this circuit allows quick gas equilibration with a t_{1/2} of 30 s, which is highly suitable for use during graded exercise protocols. The incremental exercise protocol increases in slope every 3 min until exhaustion. Exhaustion was considered to exist if a mouse spent more than a few seconds on the shock device, at which point the experiment was terminated.

Results

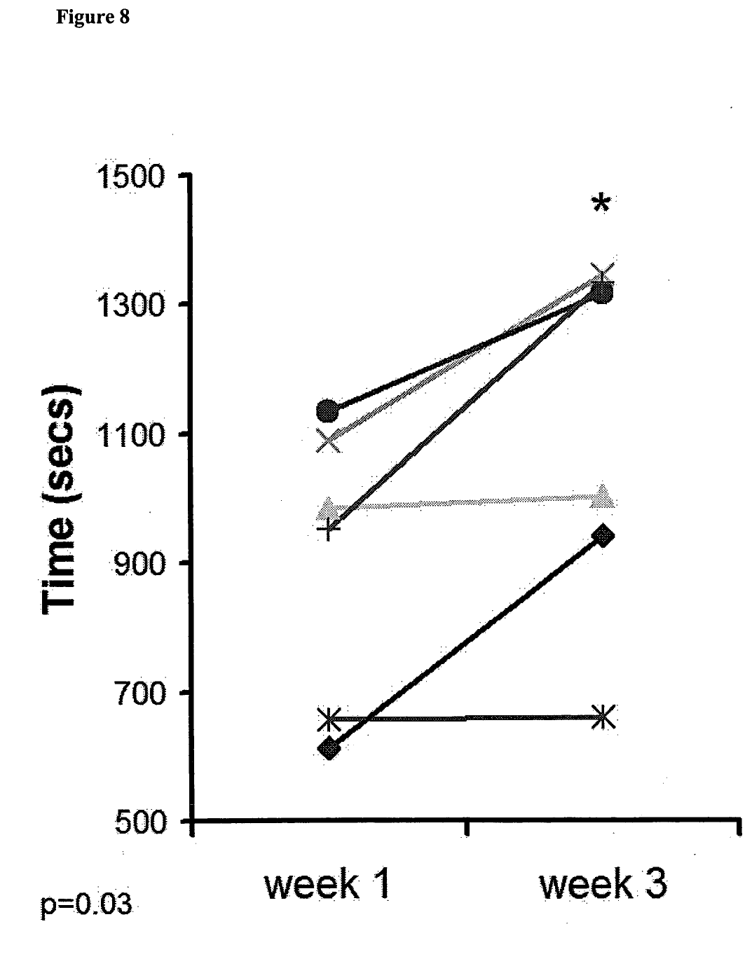

Mice (n=6) with experimentally induced heart failure were administered Pyr-apelin-13 (2 mg/kg/day) by means of an implanted osmotic pump. Mice were subjected to treadmill exercise testing at time points during weeks 1 and 3. The time points were 16-17 days apart. The data is presented in Table 5 and in FIG. 8. In Table 5, t₁ represents time to exhaustion at the first time point, and t₂ represents time to exhaustion at the second time point. In FIG. 8, the first and second data points for each mouse are indicated using identical symbols. As shown in FIG. 8, the average time to exhaustion increased markedly between weeks 1 and 3. The data was analyzed using a paired t-test. The p-value was <0.05. A control group of mice was administered saline. Average exercise capacity diminished in this group between 1 and 3 weeks (data not shown).

Additional parameters such as heart rate, blood pressure, cardiac output, plasma apelin levels, etc., can also be assessed.

TABLE 5
Exercise capacity in mice treated with apelin.

| Mouse # | Treatment | t₁ | t₂ |
|---|---|---|---|
| 1 | Apelin | 612 | 941 |
| 2 | Apelin | 985 | 1003 |
| 3 | Apelin | 1090 | 1345 |
| 4 | Apelin | 656 | 660 |
| 5 | Apelin | 1134 | 1317 |
| 6 | Apelin | 952 | 1332 |

### Equivalents

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

1. A composition comprising:
a targeting agent conjugated to a functional moiety, wherein the targeting agent selectively binds to a polypeptide encoded by a UIR or DIR gene.

2. The method of claim 1, wherein the UIR or DIR gene encodes a polypeptide selected from the group consisting of: APJ, mitogen-activated protein kinase 4, the TEC protein tyrosine kinase, the P311 protein, guanyltate binding protein 1, tumor necrosis factor (ligand) superfamily member 10, a splice variant of the regulatory domain (α subunit) of the L-type calcium channel, myosin light chain kinase pseudogene expression product, and myosin light chain 2a.

3. The composition of claim 1, wherein the targeting agent comprises an antibody or an antigen-binding antibody fragment that specifically binds to the polypeptide.

4. The composition of claim 3, wherein the targeting agent comprises a ligand that specifically binds to the polypeptide.

5. The composition of claim 1, wherein the functional moiety comprises a therapeutic agent.

6. The composition of claim 1, wherein the functional moiety comprises an imaging agent.

7. The composition of claim 6, wherein the agent is a paramagnetic, radioactive or fluorogenic ion.

8. A method of imaging cardiac tissue comprising steps of:
(i) administering to a subject an effective amount of a targeting agent that specifically binds to a UIR or DIR polypeptide, wherein the targeting agent is linked to a functional moiety that enhances detectability of cardiac cells by an imaging procedure; and
(ii) subjecting the subject to the imaging procedure.

9. The method of claim 8, wherein the targeting agent is an antibody.

10. A method of targeting a molecule selectively to a cardiac cell in culture or in a subject comprising steps of: of:
(i) conjugating the molecule to an antibody or ligand that specifically binds to a DIR polypeptide to form a conjugate; and
(ii) administering the conjugate to the cell or to the subject.

11. A method of targeting a molecule selectively to a cardiac cell in culture or in a subject comprising steps of:
(i) associating the molecule with a delivery vehicle, wherein the delivery vehicle comprises a targeting agent that specifically binds to a UIR or DIR polypeptide; and
(ii) administering the delivery vehicle to the cell or subject.

12. A method for identifying an agent that modulates expression or activity of a UIR or DIR polynucleotide or polypeptide comprising steps of:
(i) providing a sample comprising a UIR or DIR polynucleotide or polypeptide;
(ii) contacting the sample with a candidate compound;
(iii) determining whether the level of expression or activity of the polynucleotide or polypeptide in the presence of the compound is increased or decreased relative to the level of expression or activity of the polynucleotide or polypeptide in the absence of the compound; and
(iv) identifying the compound as a modulator of the expression or activity of the UIR or DIR polynucleotide or polypeptide if the level of expression or activity of the UIR or DIR polynucleotide or polypeptide is higher or lower in the presence of the compound relative to its level of expression or activity in the absence of the compound.

13. A method of providing diagnostic or prognostic information related to heart failure comprising steps of:
(i) providing a subject in need of diagnostic or prognostic information related to heart failure;
(ii) determining the level of expression or activity of a UIR or DIR polynucleotide or polypeptide, or the level of a ligand for a UIR or DIR polypeptide, in the subject or in a biological sample obtained from the subject; and
(iii) utilizing the information to provide diagnostic or prognostic information.

14. The method of claim 13, wherein the step of utilizing comprises comparing the expression level or activity of the UIR or DIR polynucleotide or polypeptide, or the level of the ligand, with predetermined ranges of values for the expression level or activity of the UIR or DIR polynucleotide or polypeptide, or predetermined ranges of values for the level of the ligand, wherein the ranges are associated with levels of risk that a subject suffers from heart failure, levels of disease severity, degree of response to treatment, or another type of diagnostic or prognostic information, thereby obtaining an indication of the risk, disease severity, or degree of response to treatment.

15. The method of claim 13, wherein the sample is a blood, plasma, or serum sample.

16. The method of claim 13, wherein the ligand is an apelin peptide.

17. A method of treating or preventing heart failure or a disease or condition associated with heart failure comprising steps of:
(i) providing a subject at risk of or suffering from heart failure or a disease or condition associated with heart failure; and
(ii) administering a composition that modulates a UIR or DIR gene.

18. The method of claim 17, wherein the composition increases the functional activity of APJ.

19. A method of treating or preventing heart failure or a disease or condition associated with heart failure comprising steps of:
(i) providing a subject at risk of or suffering from heart failure or a disease or condition associated with heart failure; and
(ii) administering a composition comprising an apelin peptide to the subject.

20. The method of claim 19, wherein the apelin peptide is administered chronically.

21. The method of claim 19, wherein the apelin peptide is administered in an amount effective to improve at least one hemodynamic parameter or prognostic variable for heart failure.

22. The method of claim 21, wherein the hemodynamic parameter is ventricular preload, ventricular afterload, contractile reserve, or cardiac output.

23. The method of claim 21, wherein the prognostic variable is exercise capacity.

24. The method of claim 21, wherein the apelin peptide is administered chronically, and wherein the hemodynamic parameter is ventricular preload, ventricular afterload, contractile reserve, or cardiac output, or wherein the prognostic variable is exercise capacity.
